SEQUENCE LISTING (27 sequences)

SEQ ID NO: 1 - protein, 12 aa, synthetic construct
  REGION 1..12: note = DPMI delta (6-10 olefin staple)-NH2
  REGION 1..12: note = MISC_FEATURE - Amino acids 1-12 are D-amino acids
  REGION 1..12: note = MISC_FEATURE - Xaa6 and Xaa10 are linked together in an olefin linkage
  SITE 1: note = MISC_FEATURE - N-terminal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - X1aa is (R)-2-amino-2-methylhept-6-enoic acid
  SITE 12: note = MISC_FEATURE - C-terminal amide group
TAXYAXXEKXLR

SEQ ID NO: 2 - protein, 12 aa, synthetic construct
  REGION 1..12: note = Acetyl-DPMI delta (6-10 olefin staple)
  REGION 1..12: note = MISC_FEATURE - Amino acids 1-12 are D-amino acids
  REGION 1..12: note = MISC_FEATURE - Xaa6 and Xaa10 are linked together in an olefin linkage
  SITE 1: note = MISC_FEATURE - N-terminal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - Xaa is (R)-2-amino-2-methylhept-6-enoic acid
  SITE 12: note = MISC_FEATURE - C-terminal amide
TAXYAXXEQXLS

SEQ ID NO: 3 - protein, 15 aa, synthetic construct
  REGION 1..15: note = Acetyl-DPMI delta (6-10 olefin staple) (K9Q, R12S, 3x(D-Ala)tail)-NH2
  REGION 1..15: note = MISC_FEATURE - Amino acids 1-15 are D-amino acids
  REGION 1..15: note = MISC_FEATURE - Xaa6 and Xaa10 are linked together in an olefin linkage
  SITE 1: note = MISC_FEATURE - N-terminal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - Xaa is (R)-2-amino-2-methylhept-6-enoic acid
  SITE 15: note = MISC_FEATURE - C-terminal amide
TAXYAXXEQXLSAAA

SEQ ID NO: 4 - protein, 18 aa, synthetic construct
  REGION 1..18: note = Acetyl-DPMI delta (6-10 olefin staple) (K9Q, R12S, 6x(D-Ala)tail)-NH2
  REGION 1..18: note = MISC_FEATURE - Amino acids 1-18 are D-amino acids
  REGION 1..18: note = MISC_FEATURE - Xaa6 and Xaa10 are linked together in an olefin linkage
  SITE 1: note = MISC_FEATURE - N-terminal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - Xaa is (R)-2-amino-2-methylhept-6-enoic acid
  SITE 18: note = MISC_FEATURE - C-terminal amide
TAXYAXXEQXLSAAAAAA

SEQ ID NO: 5 - protein, 21 aa, synthetic construct
  REGION 1..21: note = Acetyl-DPMI delta (6-10 olefin staple) (K9Q, R12S, 9x(D-Ala)tail)-NH2
  REGION 1..21: note = MISC_FEATURE - Amino acids 1-21 are D-amino acids
  REGION 1..21: note = MISC_FEATURE - Xaa6 and Xaa10 are linked together in an olefin linkage
  SITE 1: note = MISC_FEATURE - N-terminal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - Xaa is( R)-2-amino-2-methylhept-6-enoic acid
  SITE 21: note = MISC_FEATURE - C-terminal amide
TAXYAXXEQXLSAAAAAAAAA

SEQ ID NO: 6 - protein, 18 aa, synthetic construct
  REGION 1..18: note = Acetyl-DPMI delta (6-10 olefin staple) (K9Q, R12S)(D-Ala)-(D-Phe)-(D-Ala)-(D-Ala)-(D-Ala)-(D-Ala) tail)-NH2
  REGION 1..18: note = MISC_FEATURE - Amino acids 1-18 are D-amino acids
  REGION 1..18: note = MISC_FEATURE - Xaa6 and Xaa10 are linked together in an olefin linkage
  SITE 1: note = MISC_FEATURE - N-termal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - Xaa is (R)-2-amino-2-methylhept-6-enoic acid
  SITE 18: note = MISC_FEATURE - C-terminal amide
TAXYAXXEQXLSAFAAAA

SEQ ID NO: 7 - protein, 18 aa, synthetic construct
  REGION 1..18: note = Acetyl-DPMI delta (6-10 olefin staple) (K9Q, R12S,3x(D-Ala)-(D-Glu)-2x(D-Ala) tail)-NH2
  REGION 1..18: note = MISC_FEATURE - Amino acids 1-18 are D-amino acids
  REGION 1..18: note = MISC_FEATURE - Xaa6 and Xaa10 are linked together in an olefin linkage
  SITE 1: note = MISC_FEATURE - N-terminal acetyl group
  SITE 3: note = MISC_FEATURE - Xaa is 6-F-Trp
  SITE 6: note = MISC_FEATURE - Xaa is (R)-2-amino-2-methylhept-6-enoic acid
  SITE 7: note = MISC_FEATURE - Xaa is p-CF3-Phe
  SITE 10: note = MISC_FEATURE - Xaa is (R)-2-amino-2-methylhept-6-enoic acid
  SITE 18: note = MISC_FEATURE - C-terminal amide
TAXYAXXEQXLSAAAEAA

SEQ ID NO: 8 - protein, 12 aa, synthetic construct
  REGION 1..12: note = DPMI delta (5-12 olefin staple)-NH2
  REGION 1..12: note = MISC_FEATURE - Amino acids 1-12 are D-amino acids
  REGION 1..12: note = MISC_FEATURE - Xaa5 and Xaa12 are linked together in di-alkyne linkage
  SITE 1: note = MISC_FEATURE - N-terminal acetyl group
  SITE 3: note = MISC_FEATURE - Xaa is D-6-Fluoro-Trp
  SITE 5: note = MISC_FEATURE - Xaa is (S)-2-amino-2-methyldec-9-enoic acid
  SITE 7: note = MISC_FEATURE - Xaa is D-(p-CF3-Phe)
  SITE 7: note = MISC_FEATURE - Xaa is (R)-2-amino-2-methylhept-6-enoic acid
  SITE 12: note = MISC_FEATURE - C-terminal amide
TAXYXNXEKLLX

SEQ ID NO: 9 - protein, 18 aa, synthetic construct
  REGION 1..18: note = Acetyl-DPMI delta (5-12 alkyne staple) (K9Q, 6x(D-Ala) tail)-NH2
  REGION 1..18: note = MISC_FEATURE - Amino acids 1-18 are D-amino acids
  REGION 1..18: note = MISC_FEATURE - Xaa5 and Xaa12 are linked together in di-alkyne linkage
  SITE 1: note = MISC_FEATURE - N-terminal acetyl group
  SITE 3: note = MISC_FEATURE - Xaa is D-6-Fluoro-Trp
  SITE 5: note = MISC_FEATURE - Xaa is (S)-2-amino-2-methylhept-6-ynoic acid
  SITE 7: note = MISC_FEATURE - Xaa is D-(p-CF3-Phe)
  SITE 12: note = MISC_FEATURE - (S)-2-amino-2-methyloct-6-ynoic acid
  SITE 18: note = MISC_FEATURE - C-terminal amide
TAXYXNXEQLLXAAAAAA

SEQ ID NO: 10 - protein, 18 aa, synthetic construct
  REGION 1..18: note = Acetyl-DPMI delta (5-12 alkyne staple) (K9Q, (D-Glu)-5x(D-Ala)tail)-NH2
  REGION 1..18: note = MISC_FEATURE - Amino acids 1-18 are D-amino acids
  REGION 1..18: note = MISC_FEATURE - Xaa5 and Xaa12 are linked together in di-alkyne linkage
  SITE 1: note = MISC_FEATURE - N-terminal acetyl group
  SITE 3: note = MISC_FEATURE - Xaa is D-6-Fluoro-Trp
  SITE 5: note = MISC_FEATURE - Xaa is (S)-2-amino-2-methylhept-6-ynoic acid
  SITE 7: note = MISC_FEATURE - Xaa is D-(p-CF3-Phe)
  SITE 12: note = MISC_FEATURE - Xaa is (S)-2-amino-2-methyloct-6-ynoic acid
  SITE 18: note = MISC_FEATURE - C-terminal amide
TAXYXNXEQLLXEAAAAA

SEQ ID NO: 11 - protein, 12 aa, synthetic construct
  REGION 1..12: note = DPMI-delta
  SITE 3: note = MISC_FEATURE - Xaa is D-6-Fluoro-Trp
  SITE 7: note = MISC_FEATURE - Xaa is D-(p-CF3-Phe)
TAXYANXEKLLR

SEQ ID NO: 12 - protein, 379 aa, synthetic construct
  REGION 1..379: note = Human MDM2 1-125 sequence comprising a modified TEV cleavage site
MSDKIIHSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKLEVLFQGHMHHHHHHSSGVDLGTENLYFQGMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSEN

SEQ ID NO: 13 - protein, 7 aa, synthetic construct
  REGION 1..7: note = TEV cleavage site
ENLYFQS

SEQ ID NO: 14 - protein, 7 aa, synthetic construct
  REGION 1..7: note = Modified TEV cleavage site
ENLYFQG

SEQ ID NO: 15 - protein, 393 aa, synthetic construct [sequence omitted]

SEQ ID NO: 16 - protein, 15 aa, synthetic construct
  REGION 1..15: note = p53 peptide (amino acids 19-29) of native human p53
SQETFSDLWKLLPEN

SEQ ID NO: 17 - protein, 21 aa, synthetic construct
  REGION 1..21: note = A 6-10 olefin stapled p53 peptidomimetic macrocycle
  REGION 1..12: note = MISC_FEATURE - The N-terminus is optionally conjugated to an acyl group havingthe formula RCO- wherein R is an alkane having the formulaCnH2n‘wherein n is an integer from 1 to 10
  REGION 1..21: note = MISC_FEATURE - The C-terminal amino acid optionally comprises an amino group
  REGION 1..21: note = MISC_FEATURE - amino acids 1-21 are D amino acids
  REGION 1..21: note = MISC_FEATURE - the staple is an olefin obtained through ring-closing metathesisbetween Xaa6 and Xaa10
  SITE 3: note = MISC_FEATURE - Xaa is D-Trp, D-6-Fluoro-Trp, D-6-Chloro-Trp, D-6-Bromo-Trp,D-6-Iodo-Trp, D-6-methyl-Trp, D-6-cyano-Trp, D-6-hydroxy-Trp,D-6-NO2-Trp, D-7-Fluoro-Trp, D-7-Chloro-Trp, D-7-Bromo-Trp,D-7-Iodo-Trp, D-7-methyl-Trp, D-7-cyano-Trp, D-7-hydroxy-Trp,
  SITE 3: note = MISC_FEATURE - Xaa is D-6,7-Fluoro-Trp, D-6,7-Chloro-Trp, D-6,7-Bromo-Trp,D-6,7-Iodo-Trp, D-6,7-methyl-Trp, D-6,7-cyano-Trp,D-6,7-hydroxy-Trp, or D-6,7-NO2-Trp
  SITE 6: note = MISC_FEATURE - Xaa is (R)-2-amino-2-methylhept-6-enoic acid
  SITE 7: note = MISC_FEATURE - Xaa is D-p-Fluoro-Phe, D-p-Chloro-Phe, D-p-Bromo-Phe,D-p-Iodo-Phe, D-p-methyl-Phe, D-p-cyano-Phe, D-p-hydroxy-Phe,D-p-NO2-Phe, or D-p-CF3-Phe
  SITE 10: note = MISC_FEATURE - Xaa is (R)-2-amino-2-methylhept-6-enoic acid
  SITE 11: note = MISC_FEATURE - Xaa is D-Leu
  SITE 12: note = MISC_FEATURE - Xaa is D-Arg or D-Ser
  SITE 13: note = MISC_FEATURE - Xaa is D-Ala, D-Glu, or D-Gla (gamma-carboxylic glutamic acid
  SITE 14: note = MISC_FEATURE - Xaa is D-Ala
  SITE 15: note = MISC_FEATURE - Xaa is D-Ala, D-Phe, or D-Glu
  SITE 17: note = MISC_FEATURE - Xaa is D-Ala or absent
  SITE 18: note = MISC_FEATURE - Xaa is D-Ala or absent
  SITE 19: note = MISC_FEATURE - Xaa is D-Ala or absent
  SITE 20: note = MISC_FEATURE - Xaa is D-Ala or absent
  SITE 21: note = MISC_FEATURE - Xaa is D-Ala or absent
TAXYAXXEKXXXXXXXXXXXX

SEQ ID NO: 18 - protein, 21 aa, synthetic construct
  REGION 1..21: note = A 5-12 di-alkyne stapled p53 peptidomimetic macrocycle
  REGION 1..12: note = MISC_FEATURE - The N-terminus is optionally conjugated to an acyl group havingthe formula RCO-; R is an alkane having the formula CnH2n+1;nis an integer from 1 to 10
  REGION 1..21: note = MISC_FEATURE - The C-terminal amino acid optionally comprises an amino group
  REGION 1..21: note = MISC_FEATURE - Amino acids 1-21 are D amino acids
  REGION 1..21: note = MISC_FEATURE - Staple is a di-alkyne obtained through alkyne cross-couplingbetween Xaa5 and Xaa12
  SITE 3: note = MISC_FEATURE - Xaa is D-Trp, D-6-Fluoro-Trp, D-6-Chloro-Trp, D-6-Bromo-Trp,D-6-Iodo-Trp, D-6-methyl-Trp, D-6-cyano-Trp, D-6-hydroxy-Trp,D-6-NO2-Trp, D-7-Fluoro-Trp, D-7-Chloro-Trp, D-7-Bromo-Trp,D-7-Iodo-Trp, D-7-methyl-Trp, D-7-cyano-Trp, D-7-hydroxy-Trp,
  SITE 3: note = MISC_FEATURE - Xaa is D-6,7-Fluoro-Trp, D-6,7-Chloro-Trp, D-6,7-Bromo-Trp,D-6,7-Iodo-Trp, D-6,7-methyl-Trp, D-6,7-cyano-Trp,D-6,7-hydroxy-Trp, or D-6,7-NO2-Trp
  SITE 5: note = MISC_FEATURE - Xaa is (S)-2-amino-2-methyldec-9-enoic acid
  SITE 7: note = MISC_FEATURE - Xaa is D-p-Fluoro-Phe, D-p-Chloro-Phe, D-p-Bromo-Phe,D-p-Iodo-Phe, D-p-methyl-Phe, D-p-cyano-Phe, D-p-hydroxy-Phe,D-p-NO2-Phe, or D-p-CF3-Phe
  SITE 9: note = MISC_FEATURE - Xaa is D-Lys or D-Gln
  SITE 11: note = MISC_FEATURE - Xaa is D-Leu
  SITE 12: note = MISC_FEATURE - Xaa is (R)-2-amino-2-methylhept-6-enoic acid
  SITE 13: note = MISC_FEATURE - Xaa is D-Ala, D-Glu, D-Gla (gamma-carboxylic glutamic acid)
  SITE 14: note = MISC_FEATURE - Xaa is D-Ala
  SITE 15: note = MISC_FEATURE - Xaa is D-Ala, D-Phe, D-Glu
  SITE 16: note = MISC_FEATURE - Xaa is D-Ala
  SITE 17: note = MISC_FEATURE - Xaa is D-Ala or D-alpha-methyl-Glu
  SITE 18: note = MISC_FEATURE - Xaa is D-Ala
  SITE 19: note = MISC_FEATURE - Xaa is D-Ala or absent
  SITE 20: note = MISC_FEATURE - Xaa is D-Ala or absent
  SITE 21: note = MISC_FEATURE - Xaa is D-Ala or absent
TAXYXNXEXLXXXXXXXXXXX

SEQ ID NO: 19 - protein, 12 aa, synthetic construct
  REGION 1..12: note = A 5-12 di-alkyne stapled p53 peptidomimetic macrocycle
  REGION 1..12: note = MISC_FEATURE - The N-terminus is optionally conjugated to an acyl group havingthe formula RCO-; R is an alkane having the formula CnH2n+1;nis an integer from 1 to 10
  REGION 1..12: note = MISC_FEATURE - The C-terminal amino acid optionally comprises an amino group
  REGION 1..12: note = MISC_FEATURE - Amino acids 1-12 are D amino acids
  REGION 1..12: note = MISC_FEATURE - Staple is a di-alkyne obtained through alkyne cross-couplingbetween Xaa5 and Xaa12
  SITE 3: note = MISC_FEATURE - Xaa is D-Trp, D-6-Fluoro-Trp, D-6-Chloro-Trp, D-6-Bromo-Trp,D-6-Iodo-Trp, D-6-methyl-Trp, D-6-cyano-Trp, D-6-hydroxy-Trp,D-6-NO2-Trp, D-7-Fluoro-Trp, D-7-Chloro-Trp, D-7-Bromo-Trp,D-7-Iodo-Trp, D-7-methyl-Trp, D-7-cyano-Trp, D-7-hydroxy-Trp,
  SITE 3: note = MISC_FEATURE - Xaa is D-6,7-Fluoro-Trp, D-6,7-Chloro-Trp, D-6,7-Bromo-Trp,D-6,7-Iodo-Trp, D-6,7-methyl-Trp, D-6,7-cyano-Trp,D-6,7-hydroxy-Trp, or D-6,7-NO2-Trp
  SITE 5: note = MISC_FEATURE - Xaa is (S)-2-amino-2-methylhept-6-ynoic acid
  SITE 7: note = MISC_FEATURE - Xaa is D-p-Fluoro-Phe, D-p-Chloro-Phe, D-p-Bromo-Phe,D-p-Iodo-Phe, D-p-methyl-Phe, D-p-cyano-Phe, D-p-hydroxy-Phe,D-p-NO2-Phe, or D-p-CF3-Phe
  SITE 9: note = MISC_FEATURE - Xaa is D-Lys or D-Gln
  SITE 11: note = MISC_FEATURE - Xaa is D-Leu
  SITE 12: note = MISC_FEATURE - Xaa is (R)-2-amino-2-methyloct-7-ynoic acid
TAXYXNXEXLXX

SEQ ID NO: 20 - protein, 6 aa, synthetic construct
  REGION 1..6: note = Polypeptide tail; All D-amino acids
AAAAAA

SEQ ID NO: 21 - protein, 6 aa, synthetic construct
  REGION 1..6: note = Polypeptide tail; All D-amino acids
AFAAAA

SEQ ID NO: 22 - protein, 6 aa, synthetic construct
  REGION 1..6: note = Polypeptide tail; All D-amino acids
EAAAAA

SEQ ID NO: 23 - protein, 9 aa, synthetic construct
  REGION 1..9: note = Polypeptide tail; All D-amino acids
AAAAAAAAA

SEQ ID NO: 24 - protein, 14 aa, synthetic construct
  REGION 1..14: note = ATSP-7041
  REGION 1..14: note = MISC_FEATURE - Amino acids 1-14 are L-amino acids
  REGION 1..14: note = MISC_FEATURE - Xaa4 and Xaa11 are linked together in an olefin linkage
  SITE 1: note = MISC_FEATURE - N-terminal acetyl group
  SITE 4: note = MISC_FEATURE - Xaa is (R)-2-amino-2-methyldec-9-enoic acid
  SITE 10: note = MISC_FEATURE - Xaa is cyclo-butyl alanine (cba)
  SITE 11: note = MISC_FEATURE - Xaa is (S)-2-amino-2-methylhept-6-enoic acid
  SITE 14: note = MISC_FEATURE - C-terminal amide
LTFXEYWAQXXSAA

SEQ ID NO: 25 - protein, 18 aa, synthetic construct
  REGION 1..18: note = DPMI delta (5-12 olefin staple)-NH2
  REGION 1..12: note = MISC_FEATURE - Xaa5 and Xaa12 are linked together an olefin linkage
  SITE 1: note = MISC_FEATURE - N-terminal acetyl group
  SITE 3: note = MISC_FEATURE - Xaa is D-6-Fluoro-Trp
  SITE 5: note = MISC_FEATURE - Xaa is (S)-2-amino-2-methyldec-9-enoic acid
  SITE 7: note = MISC_FEATURE - Xaa is D-p-CF3-Phe
  SITE 12: note = MISC_FEATURE - Xaa is (R)-2-amino-2-methylhept-6-enoic acid
  SITE 12: note = MISC_FEATURE - C-terminal amide
TAXYXNXEKLLXAAAAAA

SEQ ID NO: 26 - protein, 9 aa, synthetic construct
  REGION 1..9: note = Peptide 13
  SITE 1: note = MISC_FEATURE - N-terminal FAM
  SITE 7: note = MISC_FEATURE - C-terminal amide
RFMDYWEGL

SEQ ID NO: 27 - protein, 21 aa, synthetic construct
  REGION 1..21: note = A 5-12 olefin stapled p53 peptidomimetic macrocycle
  SITE 1: note = MISC_FEATURE - the N-terminus amino group of the Thr at position 1 is conjugatedto an acyl group having the formula RCO-; R is an alkane havingthe formula CnH2n+1;n is an integer from 1 to 10
  REGION 1..21: note = MISC_FEATURE - The N-terminus amino group of the Thr at position 1 is conjugatedto an acyl group having the formula RCO-; R is an alkane havingthe formula CnH2n+1;n is an integer from 1 to 10
  REGION 1..21: note = MISC_FEATURE - The C-terminal amino acid optionally comprises an amino group
  REGION 1..21: note = MISC_FEATURE - Amino acids 1-21 are D amino acids
  REGION 1..21: note = MISC_FEATURE - Staple is an olefin obtained through ring-closing metathesisbetween X5 and X12
  SITE 3: note = MISC_FEATURE - Xaa is D-Trp, D-6-Fluoro-Trp, D-6-Chloro-Trp, D-6-Bromo-Trp,D-6-Iodo-Trp, D-6-methyl-Trp, D-6-cyano-Trp, D-6-hydroxy-Trp,D-6-NO2-Trp, D-7-Fluoro-Trp, D-7-Chloro-Trp, D-7-Bromo-Trp,D-7-Iodo-Trp, D-7-methyl-Trp, D-7-cyano-Trp, D-7-hydroxy-Trp,
  SITE 3: note = MISC_FEATURE - Xaa is D-6,7-Fluoro-Trp, D-6,7-Chloro-Trp, D-6,7-Bromo-Trp,D-6,7-Iodo-Trp, D-6,7-methyl-Trp, D-6,7-cyano-Trp,D-6,7-hydroxy-Trp, or D-6,7-NO2-Trp
  SITE 5: note = MISC_FEATURE - Xaa is (S)-2-amino-2-methyldec-9-enoic acid
  SITE 7: note = MISC_FEATURE - Xaa is D-p-Fluoro-Phe, D-p-Chloro-Phe, D-p-Bromo-Phe,D-p-Iodo-Phe, D-p-methyl-Phe, D-p-cyano-Phe, D-p-hydroxy-Phe,D-p-NO2-Phe, or D-p-CF3-Phe
  SITE 9: note = MISC_FEATURE - Xaa is D-Lys or D-Gln
  SITE 11: note = MISC_FEATURE - Xaa is D-Leu
  SITE 12: note = MISC_FEATURE - Xaa is (R)-2-amino-2-methylhept-6-enoic acid
  SITE 13: note = MISC_FEATURE - Xaa is D-Ala, D-Glu, D-Gla (gamma-carboxylic glutamic acid)
  SITE 14: note = MISC_FEATURE - Xaa is D-Ala
  SITE 15: note = MISC_FEATURE - Xaa is D-Ala, D-Phe, or D-Glu
  SITE 16: note = MISC_FEATURE - Xaa is D-Ala
  SITE 17: note = MISC_FEATURE - Xaa is D-Ala or D-alpha-methyl-Glu
  SITE 18: note = MISC_FEATURE - Xaa is D-Ala
  SITE 19: note = MISC_FEATURE - Xaa is D-Ala or absent
  SITE 20: note = MISC_FEATURE - Xaa is D-Ala or absent
  SITE 21: note = MISC_FEATURE - Xaa is D-Ala or absent
TAXYXNXEXLXXXXXXXXXXX

(19) United States
(12) Patent Application Publication
(10) Pub. No.: US 2025/0042961 A1
(43) Pub. Date: Feb. 6, 2025

(54) P53 PEPTIDOMIMETIC MACROCYCLES

(71) Applicants: MERCK SHARP & DOHME LLC, RAHWAY, NJ (US); MSD International GMBH, Lucerne (CH); Agency for Science, Technology and Research, Singapore (SG)

(72) Inventors: Hubert Josien, Jersey City, NJ (US); Arun Chandramohan, Tamil Nadu (IN); Charles William Johannes, Cambridge, MA (US); Christopher J. Brown, Singapore (SG); Srinivasaraghavan Kannan, Bangalore (IN); Anthony William Partridge, Cambridge, MA (US); Chandra Shekhar Verma, Singapore (SG); Lin Yan, East Brunswick, NJ (US); Tsz Ying Yuen, Singapore (SG)

(21) Appl. No.: 18/716,399

(22) PCT Filed: Dec. 5, 2022
(86) PCT No.: PCT/US2022/051762

§ 371 Date: Jun. 4, 2024

Related U.S. Application Data
(60) Provisional application No. 63/288,204, filed on Dec. 10, 2021.

(51) Int. Cl. C07K 14/47; A61K 31/203; A61K 31/4745; A61K 31/475; A61K 31/495; A61K 31/675; A61K 31/7048; A61K 31/7068; A61K 33/243; A61K 38/00; A61K 38/12; A61K 38/14; A61K 39/395

(52) U.S. Cl. CPC C07K 14/4746; A61K 31/203; A61K 31/4745; A61K 31/475; A61K 31/495; A61K 31/675; A61K 31/7048; A61K 31/7068; A61K 33/243; A61K 38/12; A61K 38/14; A61K 39/3955; A61K 38/00

(57) ABSTRACT

Disclosed are p53 peptidomimetic macrocycles, each p53 peptidomimetic macrocycle comprising an i, i+4 olefin staple and a polypeptide tail covalently linked to the p53 peptidomimetic macrocycle; an i, i+7 olefin staple and a polypeptide tail covalently linked to the p53 peptidomimetic macrocycle; or, an i, i+7 di-alkyne staple and optionally a polypeptide tail covalently linked to the p53 peptidomimetic macrocycle; wherein the p53 peptidomimetic macrocycle comprises all D-configuration amino acids and the polypeptide tail comprises three to nine amino acids, each amino acid of the polypeptide tail independently having a D-configuration or an L-configuration. The p53 peptidomimetic macrocycles are protease resistant, cell permeable without inducing membrane disruption, and intracellularly activate p53 by binding MDM2 and MDMX, thereby antagonizing MDM2 and MDMX binding to p53. These p53 peptidomimetic macrocycles may be useful in anticancer therapies, particularly in combination with chemotherapy or radiation therapy.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 3, 2022, is named 25346WOPCT_SL.XML and is 104 bytes in size.

### BACKGROUND OF THE INVENTION

### (1) Field of the Invention

The present invention provides p53 peptidomimetic macrocycles, each p53 peptidomimetic macrocycle comprising an i, i+4 olefin staple and a polypeptide tail covalently linked to the p53 peptidomimetic macrocycle; an i, i+7 olefin staple and a polypeptide tail covalently linked to the p53 peptidomimetic macrocycle; or an i, i+7 di-alkyne staple and optionally a polypeptide tail covalently linked to the p53 peptidomimetic macrocycle; wherein the p53 peptidomimetic macrocycle comprises all D-configuration amino acids and the polypeptide tail comprises three to nine amino acids, each amino acid of the polypeptide tail independently having a D-configuration or an L-configuration. The p53 peptidomimetic macrocycles are protease-resistant, cell-permeable without inducing membrane disruption, and intracellularly activate p53 by binding MDM2 and MDMX, thereby antagonizing MDM2 and MDMX binding to p53.

### (2) Description of Related Art

p53 is a key tumor suppressor protein that primarily functions as a DNA transcription factor. It is commonly abrogated in cancer and plays a crucial role in guarding the cell in response to various stress signals through the induction of cell cycle arrest, apoptosis, or senescence [46]. Mechanisms that frequently result in the inactivation of p53 and tumorigenesis include increased expression of the p53-negative regulators MDM2 and MDMX (aka MDM4). Both MDM2 and MDMX attenuate p53 function by interacting directly with p53 and preventing its interaction with the relevant activation factors required for transcription, e.g., dTAF_{II}, hTAF_{II}. In addition, they are both E3 ligase components and target p53 for proteosomal-mediated degradation. MDMX, unlike MDM2, has no intrinsic E3 ubiquitin ligase activity. Instead, MDMX forms heterodimeric complexes with MDM2 whereby it stimulates the ubiquitin activity of MDM2. As a result, p53 activity and protein levels are acutely suppressed by MDM2 and MDMX overexpression. Development of inhibitors to disrupt the interactions of p53 with either MDM2 or MDMX, or both, are therefore highly desirable as they will prevent p53 degradation and restore a p53 dependent transcriptional anti-tumor response [47,48].

The structural interface of the p53 MDM2/MDMX complex is characterized by an α-helix from the N-terminal transactivation domain of p53 which binds into a hydrophobic groove on the surface of the N-terminal domain of both MDM2 and MDMX. Three hydrophobic residues, Phe¹⁹, Trp²³ and Leu²⁶, of p53 are critical determinants of this interaction and project deeply into the MDM2/MDMX interaction groove [See FIG. 1A]. The isolated p53 peptide is largely disordered, morphing into an a-helical conformation upon binding. There are several examples of small molecules, peptides, and biologics that mimic these interactions and compete for MDM2/MDMX binding, with the release of p53 [49]. However, a large majority of the small molecules developed exhibit little affinity and activity against MDMX, which possesses several distinct structural differences in the p53 peptide binding groove compared to MDM2. Although several MDM2 specific molecules have entered initial clinical trials, they have largely been met with dose limiting toxicities in patients [49]. Overexpression of MDMX in tumors has been demonstrated to attenuate the effectiveness of MDM2 specific compounds, presumably through the maintenance of heterodimeric complexes of MDM2 and MDMX that inhibit and target p53 for proteosomal degradation. MDM2-selective inhibitors may also induce higher levels of MDMX. This highlights the importance of targeting both proteins simultaneously to achieve efficient activation of p53 to achieve an optimal therapeutic response.

Protein-protein interactions (PPIs) are central to most biological processes and are often dysregulated in disease [1, 2]. Therefore, PPIs are attractive therapeutic targets for novel drug discovery. However, in contrast to the deep protein cavities that typically accommodate small molecules, PPI surfaces are generally large and flat, and this has contributed to the limited successful development of small molecule inhibitors for PPI targets [3]. The realization that 40% of all PPIs are mediated by relatively short peptide motifs gave rise to the possibility of developing peptide-based inhibitors that would compete orthosterically for the interface between ligand-target cognate partners [4]. When taken out of the protein ligand context and synthesized, such peptides may often be unstructured and intrinsically disordered, yet capable to achieve their biologically relevant conformation upon protein target binding [4]. However, for intracellular targets, the peptide modality may be challenging due to proteolytic sensitivity, low conformational stability (yielding weak affinities and off target effects), and poor cell permeability (further limiting prosecution of intracellular targets and/or oral bioavailability) [5-11]. To address these issues, several strategies have been pursued, including macrocyclization and modifications of the peptide backbone to yield molecules with improved activities and pharmacokinetic properties as well as constraining the peptide into to its biologically relevant conformation to bind its target) [5-13]. First, by biasing the peptides toward their bound conformations, entropic penalties upon binding are reduced, thus improving binding constants as well as presumably decreasing the opportunity for unwanted off-target effects. Secondly, macrocyclization may confer varying degrees of proteolytic resistance by modifying key backbone and/or side-chain structural moieties in the peptide. Thirdly, macrocyclization may enhance cell permeability, such as through increased stability of intramolecular hydrogen bonding to reduce the desolvation penalty otherwise incurred in the transport of peptides cross an apolar cell membrane.

Amongst the several cyclization techniques described, stapling via metathesis using a non-proteogenic amino acid such as alpha methyl alkenyl side chains has been proven to be very effective [13-18], particularly when the desired secondary structure of the peptide macrocycle is helical. Stapling requires incorporation of the appropriate unnatural amino acid precursors to be placed at appropriate locations along the peptide sequence such that they do not interfere with the binding face of the helix. Although they have largely been used to stabilize helical conformations, recent studies have also applied ring-closing metathesis (RCM) strategies to non-helical peptides [19, 20].

The stapled peptide strategy has been successfully applied to inhibit several PPIs of therapeutic potential including, BCL-2 family-BH3 domains [21-24], β-catenin-TCF [25], Rab-GTPase-Effector [26], ERa-coactivator protein [27], Cullin3-BTB [28], VDR-coactivator protein [29], eIf4E [30], ATSP-7041 [See WO2013123266], SAH-p53-8 [Bemal et al., Cancer Cell 18: 411-422 (2010)], and p53-MDM2/MDMX [31-34]. Noteworthy, in the case of p53-MDM2/MDMX, a dual selective stapled peptide (ALRN-6924; Aileron Therapeutics, Inc.) has been further successfully advanced to phase II clinical trials [35-37]. Although this example is unquestionably encouraging for the advancement of stapled peptides into the clinic, challenges yet remain. Amongst these, engineering molecules with sufficient proteolytic stability for sustained target binding and cellular activity is critical. Indeed, although stapling L-amino acid peptides can confer resistance to protease-mediated degradation, the effect is often not complete, and may affect residues located outside of the macrocycle [38-40].

On the other hand, all-D configuration α-amino acid peptides are hyper-stable against proteolysis as most proteases are chiral, they distinguish between L- and D-enantiomeric versions of the substrate; as a result, all-D configuration α-amino acid peptides are able to resist the activity of proteases. All-D configuration α-amino acid peptides have been engineered with strong binding affinity against a variety of targets including p53-MDM2 [41-42], VEGF-VEGF-receptor [43], PD-1-PD-L1 [44], and human immunodeficiency virus type 1 (HIV-1) entry [45]. Unfortunately, although all-D configuration α-amino acid peptides are intrinsically hyper-stable to proteolysis, the generally lack membrane permeability and cellular activity.

For example, ^{D}PMI-δ, is an all-D configuration α-amino acid linear peptide (PMI refers to p53-MDM2/MDMX inhibitor) that was derived from a mirror image phage display screen reported by Liu et al. [41] and in U.S. Pub. Patent No. 20120328692. However, this peptide lacked cell permeability, but did activate p53 in cells when delivered using nano-carriers [42].

Peptide-based inhibitors hold great potential for targeted modulation of intracellular protein-protein interactions (PPIs) since they can access a huge range of chemical space including sequence diversity as well as a host of different secondary and tertiary structures. Currently, however, liabilities hinder the development of peptide therapeutics including poor conformational stability, proteolytic sensitivity and cell permeability. In addition, translation of these in vitro binders into intracellularly active and in vivo active compounds with on target selectivity and specificity is difficult. Several contemporary peptide design strategies address these issues to different degrees. Strategic macrocyclization through optimally placed chemical braces such as an olefin hydrocarbon crosslinks, commonly referred to as staples, can address these issues by i) restricting conformational freedom to give improved target affinities, ii) improving proteolytic resistance, and iii) enhancing cell permeability. Conversely, molecules constructed entirely from D-amino acids are hyper-resistant to proteolytic cleavage but generally lack conformational stability and membrane permeability.

### SUMMARY OF THE INVENTION

We have developed p53 peptidomimetic macrocycles that resolve many of the issues with peptide inhibitors identified in the art. The present invention provides p53 peptidomimetic macrocycles, each p53 peptidomimetic macrocycle comprising an i, i+4 olefin staple and a polypeptide tail covalently linked to the p53 peptidomimetic macrocycle; an i, i+7 olefin staple and a polypeptide tail covalently linked to the p53 peptidomimetic macrocycle; or an i, i+7 di-alkyne staple and optionally a polypeptide tail covalently linked to the p53 peptidomimetic macrocycle; wherein the p53 peptidomimetic macrocycle comprises all D-configuration amino acids (D-amino acids) and the polypeptide tail comprises three to nine amino acids, each amino acid of the polypeptide tail independently having a D-configuration or an L-configuration or, in specific embodiments, each amino acid in the polypeptide tail has a D-configuration. These p53 peptidomimetic macrocycles (i) are protease resistant, (ii) have conformational stability, (iii) are cell-permeable without inducing membrane disruption, and (iv) have low or no cellular toxicity (the disclosed p53 peptidomimetic macrocycles reduced cellular activity with counter-screen activity and lactate dehydrogenase (LDH) release). Furthermore, the p53 peptidomimetic macrocycles of the present invention bind mouse double minute 2 (MDM2 aka E3 ubiquitin-protein ligase) and MDMX (aka MDM4) and intracellularly activate p53 by binding MDM2 and MDMX, thereby antagonizing MDM2 and MDMX binding to p53.

In particular embodiments, the present invention provides a p53 peptidomimetic macrocycle comprising an i, i+4 olefin staple and a polypeptide tail covalently linked at its N-terminus to the C-terminal amino acid of the p53 peptidomimetic macrocycle; an i, i+7 olefin staple and a polypeptide tail covalently linked at its N-terminus to the C-terminal amino acid of the p53 peptidomimetic macrocycle; or, an i, i+7 di-alkyne staple and optionally a polypeptide tail covalently linked at its N-terminus to the C-terminal amino acid of the p53 peptidomimetic macrocycle; wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

In particular embodiments, the aforementioned p53 peptidomimetic macrocycle comprises an i, i+4 olefin staple and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

In particular embodiments, the aforementioned p53 peptidomimetic macrocycle comprises an i, i+7 olefin staple and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration. In specific embodiments, the olefin staple is an alkyne staple.

In particular embodiments, the aforementioned p53 peptidomimetic macrocycle comprises an i, i+7 di-alkyne staple and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

In further embodiments, the present invention provides a p53 peptidomimetic macrocycle comprising an i, i+4 olefin staple formed between the α-carbons of two α,α-disubstituted amino acids located at amino acid positions 6 and 10 of the p53 peptidomimetic macrocycle (such staple a “6-10 olefin staple”) and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration. In particular embodiments, the α,α-disubstituted amino acids at positions 6 and 10 comprise (R)-2-amino-2-methylhept-6-enoic acid.

In further embodiments, the present invention provides a p53 peptidomimetic macrocycle comprising 12 amino acids and an i, i+4 olefin staple formed between the α-carbons of two α,α-disubstituted amino acids located at amino acid positions 6 and 10 of the p53 peptidomimetic macrocycle and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration. In particular embodiments, the α,α-disubstituted amino acids at amino acid positions 6 and 10 comprise ((R)-2-amino-2-methylhept-6-enoic acid. In particular embodiments of the p53 peptidomimetic macrocycle comprising the 6-10 olefin staple, the p53 peptidomimetic macrocycle further comprises D-6-fluoro-tryptophane at position 3 and D-p-CF₃-phenylalanine at position 7.

In a further still embodiment of the p53 peptidomimetic macrocycle comprising the 6-10 olefin staple, the amino acid at position 1 comprises threonine, the amino acid at position 2 comprises alanine, the amino acid at position 4 comprises tyrosine, the amino acid at position 5 comprises alanine, the amino acid at position 8 comprises glutamic acid, the amino acid at position 9 comprises lysine or glutamine, the amino acid at position 11 comprises leucine, and the amino acid at position 12 comprises arginine or serine.

In a further embodiment of the p53 peptidomimetic macrocycle comprising the 6-10 olefin staple, the amino acid at position 9 comprises glutamine and the amino acid at position 12 comprises serine.

In further embodiments, the present invention provides p53 peptidomimetic macrocycle comprising a 5-12 di-alkyne staple formed between the α-carbons of two α,α-disubstituted amino acids located at amino acid positions 5 and 12 of the p53 peptidomimetic macrocycle and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

In particular embodiments of the p53 peptidomimetic macrocycle comprising a 5-12 di-alkyne staple, the α,α-disubstituted amino acid at amino acid position 5 comprises (S)-2-amino-2-methylhept-6-ynoic acid and the α,α-disubstituted amino acid at amino acid position 12 comprises (R)-2-amino-2-methyloct-7-ynoic acid.

In particular embodiments of the p53 peptidomimetic macrocycle comprising a 5-12 di-alkyne staple, the above p53 peptidomimetic macrocycle is covalently linked at the C-terminal amino acid to the N-terminus of a polypeptide tail, wherein the polypeptide tail comprises three to nine amino acids, each amino acid of the polypeptide tail independently having a D-configuration or an L-configuration or, in specific embodiments, each amino acid in the polypeptide tail having a D-configuration.

In further embodiments, the present invention provides a p53 peptidomimetic macrocycle that comprises 12 amino acids and an i, i+7 di-alkyne staple formed between the α-carbons of two α,α-disubstituted amino acids located at amino acid positions 5 and 12 of the p53 peptidomimetic macrocycle (such staple a 5-12 di-alkyne staple’), and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration.

In particular embodiments of the p53 peptidomimetic macrocycle comprising a 5-12 di-alkyne staple, the α,α-disubstituted amino acid at amino acid position 5 comprises (S)-2-amino-2-methylhept-6-ynoic acid and the α,α-disubstituted amino acid at amino acid position 12 comprises (R)-2-amino-2-methyloct-7-ynoic acid.

In particular embodiments of the p53 peptidomimetic macrocycle comprising a 5-12 di-alkyne staple, the p53 peptidomimetic macrocycle is covalently linked at the C-terminal amino acid to the N-terminus of a polypeptide tail, wherein the polypeptide tail comprises three to nine amino acids, each amino acid of the polypeptide tail independently having a D-configuration or an L-configuration or, in specific embodiments, each amino acid in the polypeptide tail having a D-configuration.

In particular embodiments of the p53 peptidomimetic macrocycle comprising a 5-12 di-alkyne staple, the p53 peptidomimetic macrocycle comprising a 5-12 di-alkyne staple further comprises D-6-fluoro-tryptophane at amino acid position 3 and D-p-CF₃-phenylalanine at amino acid position 7.

In a further still embodiment of the p53 peptidomimetic macrocycle comprising a 5-12 di-alkyne staple, the amino acid at position 1 comprises threonine, the amino acid at position 2 comprises alanine, the amino acid at position 4 comprises tyrosine, the amino acid at position 6 comprises asparagine, the amino acid at position 8 comprises glutamic acid, the amino acid at position 9 comprises lysine or glutamine, the amino acid at position 10 comprises leucine, and the amino acid at position 11 comprises leucine. In a further embodiment, the amino acid at position 9 comprises glutamine.

In further embodiments, the present invention provides a p53 peptidomimetic macrocycle comprising an i, i+7 olefin staple formed between the α-carbons of two α,α-disubstituted amino acids located at amino acid positions 5 and 12 of the p53 peptidomimetic macrocycle (such staple a “5-12 olefin staple”) and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration, and wherein the α,α-disubstituted amino acid at amino position 5 is (S)-2-amino-2-methyldec-9-enoic acid and the α,α-disubstituted amino acid at amino acid position 12 is (R)-2-amino-2-methylhept-6-enoic acid.

In particular embodiments of the p53 peptidomimetic macrocycle comprising the 5-12 olefin linkage, the p53 peptidomimetic macrocycle is covalently linked at the C-terminal amino acid to the N-terminus of a polypeptide tail, wherein the polypeptide tail comprises three to nine amino acids, each amino acid of the polypeptide tail independently having a D-configuration or an L-configuration or, in specific embodiments, each amino acid in the polypeptide tail having a D-configuration.

In further embodiments, the present invention provides a p53 peptidomimetic macrocycle that comprises 12 amino acids and an i, i+7 olefin staple formed between the α-carbons of two α,α-disubstituted amino acids located at amino acid positions 5 and 12 of the p53 peptidomimetic macrocycle and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration, and wherein the α,α-disubstituted amino acid at amino position 5 is 2-amino-2-methyldec-9-enoic acid and the α,α-disubstituted amino acid at amino acid position 12 is (R)-2-amino-2-methylhept-6-enoic acid.

In particular embodiments of the p53 peptidomimetic macrocycle comprising the 5-12 olefin linkage, the p53 peptidomimetic macrocycle is covalently linked at the C-terminal amino acid to the N-terminus of a polypeptide tail, wherein the polypeptide tail comprises three to nine amino acids, each amino acid of the polypeptide tail independently having a D-configuration or an L-configuration or, in specific embodiments, each amino acid in the polypeptide tail having a D-configuration.

In particular embodiments of the p53 peptidomimetic macrocycle comprising the 5-12 olefin linkage, the p53 peptidomimetic macrocycle further comprises D-6-fluoro-tryptophane at amino acid position 3 and D-p-CF₃-phenylalanine at amino acid position 7.

In a further still embodiment of the p53 peptidomimetic macrocycle comprising the 5-12 olefin linkage, the amino acid at position 1 comprises threonine, the amino acid at position 2 comprises alanine, the amino acid at position 4 comprises tyrosine, the amino acid at position 6 comprises asparagine, the amino acid at position 8 comprises glutamic acid, the amino acid at position 9 comprises lysine or glutamine, the amino acid at position 10 comprises leucine, and the amino acid at position 11 comprises leucine. In a further embodiment, the amino acid at position 9 comprises glutamine.

In particular embodiment of the p53 peptidomimetic macrocycles disclosed herein, one substituent of the α,α-disubstituted amino acids is an alkenyl and the other substituent of the α,α-disubstituted amino acids is a methyl. In particular embodiments, the alkenyl is a C³⁻¹⁰ alkenyl, a C⁴⁻⁷ alkenyl, or a C⁵⁻⁶ alkenyl.

The present invention further provides a method for converting a p53 peptidomimetic macrocycle and an i, i+4 olefin linkage into a p53 peptidomimetic macrocycle having improved pharmacological properties of low or undetectable toxicity, conformational stability, and membrane permeability comprising covalently linking to the C-terminal amino acid of the p53 peptidomimetic macrocycle a polypeptide tail comprising three to nine amino acids, each amino acid of the polypeptide tail independently having a D-configuration or an L-configuration or, in specific embodiments, each amino acid in the polypeptide tail having a D-configuration. In a further embodiment, the α-carbons of two α,α-disubstituted amino acids at positions 6 and 10 of the p53 peptidomimetic macrocycles are linked in the i, i+4 olefin linkage. In particular embodiments, the α,α-disubstituted amino acids at amino acid positions 6 and 10 comprise ((R)-2-amino-2-methylhept-6-enoic acid.

The present invention further provides a method for converting a p53 peptidomimetic macrocycle and an i, i+7 olefin linkage or i, i+7 di-alkyne linkage into a macrocycle having improved pharmacological properties of low or undetectable toxicity, conformational stability, and membrane permeability comprising linking to the C-terminal amino acid of the p53 peptidomimetic macrocycle a polypeptide tail comprising three to nine amino acids, each amino acid of the polypeptide tail independently having a D-configuration or an L-configuration or, in specific embodiments, each amino acid in the polypeptide tail having a D-configuration. In a further embodiment, the α-carbons of two α,α-disubstituted amino acids at positions 5 and 12 of the p53 peptidomimetic macrocycles are linked in the i, i+7 olefin linkage. In particular embodiments, the α,α-disubstituted amino acid at amino acid position 5 comprises (S)-2-amino-2-methylhept-6-ynoic acid and the α,α-disubstituted amino acid at amino acid position 12 comprises (R)-2-amino-2-methyloct-7-ynoic acid.

In a further embodiment of the method, a p53 peptidomimetic macrocycle is converted into a p53 peptidomimetic macrocycle having improved pharmacological properties of low or undetectable toxicity, conformational stability, and membrane permeability by linking the α-carbons of two α,α-disubstituted amino acids located at amino acid positions 5 and 12 of the p53 peptidomimetic macrocycle in an olefin linkage and covalently linking to the C-terminal amino acid of the p53 peptidomimetic macrocycle a polypeptide tail comprising three to nine amino acids, each amino acid of the polypeptide tail independently having a D-configuration or an L-configuration or, in specific embodiments, each amino acid in the polypeptide tail having a D-configuration, wherein the α,α-disubstituted amino acid at amino position 5 is (S)-2-amino-2-methyldec-9-enoic acid and the α,α-disubstituted amino acid at amino acid position 12 is (R)-2-amino-2-methylhept-6-enoic acid.

In embodiments in which the objective is to improve binding of the p53 peptidomimetic macrocycle to MDM2 and/or MDMX and improve cellular activity in a p53 cellular reporter gene assay, the amino acid at each position of the polypeptide tail comprising three to nine amino acids is independently selected from natural or unnatural L- or D-amino acids. In particular embodiments of the disclosed p53 peptidomimetic macrocycles, the amino acid at each position of the polypeptide tail comprises alanine. In a further embodiment, the amino acids at positions 3 and 6 of the polypeptide tail are each independently selected from alkyl or aromatic amino acids. In particular embodiments of the polypeptide tail, the amino acids at positions 3 and 6 of the polypeptide tail are each independently selected from alkyl or Phenylalanine. In particular embodiments of the polypeptide tail, the polypeptide tail comprises or consists of six amino acids. In particular embodiments of the polypeptide tail, the alkyl amino acid of the polypeptide tail comprises alanine. In particular embodiments of the polypeptide tail, the polypeptide tail comprises six amino acids. In further embodiments of the polypeptide tail, each amino acid of the polypeptide tail is in the D configuration.

In embodiments in which the objective is to improve amphipathicity, solubility and/or cellular activity of the p53 peptidomimetic macrocycle, the amino acid at each position of the polypeptide tail comprising three to nine amino acids is independently selected from natural or unnatural L- or D-negatively charged amino acids with the proviso that at least one amino acid is negatively charged. In a further embodiment, of the polypeptide tail, the amino acid at position 1 of the polypeptide tail is independently selected from alkyl, glutamic acid, or gamma-carboxylic glutamic acid (Gla); the amino acid at position 3 of the polypeptide tail is independently selected from alkyl or glutamic acid; the amino acid at position 5 of the polypeptide tail is independently selected from alkyl and alpha-methyl glutamic acid, with the proviso that at least one of the amino acids at positions 1, 3, or 5 comprises glutamic acid or alpha-methyl glutamic acid. In particular embodiments of the polypeptide tail, the polypeptide tail comprises or consists of six amino acids. In further embodiments of the polypeptide tail, the alkyl amino acid of the polypeptide tail comprises alanine. In further embodiments of the polypeptide tail, each amino acid of the polypeptide tail is in the D configuration.

The present invention further provides a p53 peptidomimetic macrocycle comprising:

(SEQ ID NO: 17)
TAX³YAX⁶X⁷EKX¹⁰X¹¹X¹²X¹³X¹⁴X¹⁵X¹⁶X¹⁷X¹⁸-X¹⁹X²⁰X²¹

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁶ is (R)-2-amino-2-methylhept-6-enoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X¹⁰ is (R)-2-amino-2-methylhept-6-enoic acid; X¹¹ is D-Leu; X¹² is D-Arg or D-Ser; X¹³ is D-Ala, D-Glu, or D-Gla (γ-carboxylic glutamic acid); X¹⁴ is D-Ala; X¹⁵ is D-Ala, D-Phe, or D-Glu; X¹⁶ is D-Ala or absent; X¹⁷ is D-Ala, D-α-methyl-Glu, or absent; X¹⁸ is D-Ala or absent; X¹⁹ is an D-Ala or absent; X²⁰ is D-Ala or absent; X²¹ is D-Ala or absent; the N-terminal amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; and the staple is an olefin obtained through ring-closing metathesis between X⁶ and X¹⁰.

In particular embodiments, amino acids 1-12 are all D amino acids and amino acids 13-21 are each independently an L-amino acid or D-amino acid or are each a D-amino acid.

In a further embodiment of the p53 peptidomimetic macrocycle, X¹² is D-Ser. In further embodiments of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

The present invention provides a p53 peptidomimetic macrocycle comprising:

(SEQ ID NO: 18)
TAX³YX⁵NX⁷EX⁹LX¹¹X¹²X¹³X¹⁴X¹⁵X¹⁶X¹⁷X¹⁸-X¹⁹X²⁰X²¹

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁵ is (S)-2-amino-2-methylhept-6-ynoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X⁹ is D-Lys or D-Gln; X¹¹ is D-Leu; X¹² is (R)-2-amino-2-methyloct-7-ynoic acid; X¹³ is D-Ala, D-Glu, D-Gla (γ-carboxylic glutamic acid) or absent; X¹⁴ is D-Ala or absent; X¹⁵ is D-Ala, D-Phe, or D-Glu or absent; X¹⁶ is D-Ala or absent; X¹⁷ is D-Ala, D-α-methyl-Glu, or absent; XV is D-Ala or absent; X¹⁹ is D-Ala or absent; X²⁰ is D-Ala or absent; X²¹ is D-Ala or absent; the N-terminus amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; and the staple is a di-alkyne obtained through alkyne cross-coupling between X⁵ and X¹².

In particular embodiments, amino acids 1-12 are all D amino acids and amino acids 13-21 are each independently an L-amino acid or D-amino acid or are each a D-amino acid.

In a further embodiment of the p53 peptidomimetic macrocycle, X⁹ is D-Gln. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

The present invention provides a p53 peptidomimetic macrocycle comprising:

(SEQ ID NO: 27)
TAX³YX⁵NX⁷EX⁹LX¹¹X¹²X¹³X¹⁴X¹⁵X¹⁶X¹⁷X¹⁸X¹⁹X²⁰X²¹

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁵ is (S)-2-amino-2-methyldec-9-enoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X⁹ is D-Lys or D-Gln; X¹¹ is D-Leu; X¹² is (R)-2-amino-2-methylhept-6-enoic acid; X¹³ is D-Ala, D-Glu, D-Gla (γ-carboxylic glutamic acid); X¹⁴ is D-Ala; X¹⁵ is D-Ala, D-Phe, or D-Glu; X¹⁶ is D-Ala; X¹⁷ is D-Ala or D-α-methyl-Glu; X¹⁸ is D-Ala; X¹⁹ is D-Ala or absent; X²⁰ is D-Ala or absent; X²¹ is D-Ala or absent; the N-terminus amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; amino acids 1-12 are D amino acids; and the staple is an olefin obtained through ring-closing metathesis between X⁵ and X¹².

In particular embodiments, amino acids 1-12 are all D amino acids and amino acids 13-21 are each independently an L-amino acid or D-amino acid or are each a D-amino acid.

In a further embodiment of the p53 peptidomimetic macrocycle, X⁹ is D-Gln. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

The present invention provides a p53 peptidomimetic macrocycle comprising:

(SEQ ID NO: 19)
TAX³YX⁵NX⁷EX⁹LX¹¹X¹²

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁵ is (S)-2-amino-2-methylhept-6-ynoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X⁹ is D-Lys or D-Gln; X₁₁ is D-Leu; X¹² is (R)-2-amino-2-methyloct-7-ynoic acid; the N-terminus amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; amino acids 1-12 are D amino acids; and, the staple is a di-alkyne obtained through alkyne cross-coupling between X⁵ and X¹².

In a further embodiment of the p53 peptidomimetic macrocycle, X⁹ is D-Gln. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a composition comprising a p53 peptidomimetic macrocycle disclosed herein and a pharmaceutically acceptable carrier. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a method for treating cancer in a subject in need thereof comprising administering to the subject a p53 peptidomimetic macrocycle disclosed herein or a composition comprising said p53 peptidomimetic macrocycle. The present invention further provides a use of a peptidomimetic macrocycle disclosed herein for the preparation of a medicament for treating cancer. The present invention further provides a peptidomimetic macrocycle disclosed herein for the treatment of cancer. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

In further embodiments of any disclosed method, use or composition for use, the cancer is selected from the group consisting of melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, salivary cancer, pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, and cancer of hematological tissues.

The present invention further provides a method of modulating the activity of p53 and/or MDM2 and/or MDMX in a subject comprising administering to the subject a p53 peptidomimetic macrocycle disclosed herein, or a composition comprising said p53 peptidomimetic macrocycle. The present invention further provides the use of a peptidomimetic macrocycle disclosed herein for the preparation of a medicament for modulating said activity. The present invention further provides a peptidomimetic macrocycle disclosed herein for the modulation of said activity.

In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a method of antagonizing the interaction between p53 and MDM2 and/or between p53 and MDMX in a subject comprising administering to the subject a p53 peptidomimetic macrocycle disclosed herein, or a composition comprising said p53 peptidomimetic macrocycle. The present invention further provides the use of a p53 peptidomimetic macrocycle disclosed herein for the preparation of a medicament for antagonizing the interaction between p53 and MDM2 and/or between p53 and MDMX.

The present invention further provides a peptidomimetic macrocycle disclosed herein for antagonizing the interaction between p53 and MDM2 and/or between p53 and MDMX. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a combination therapy for treating cancer comprising administering to a subject a therapeutically effective amount of a p53 peptidomimetic macrocycle disclosed herein or a composition comprising said p53 peptidomimetic macrocycle and a therapeutically effective dose of a chemotherapy agent or radiation. In a further embodiment, the chemotherapy agent or radiation is administered to the subject followed by administration of the p53 peptidomimetic macrocycle; the p53 peptidomimetic macrocycle is administered to the subject followed by administration of the chemotherapy agent or radiation; or the chemotherapy agent or radiation is administered to the subject simultaneously with administration of the p53 peptidomimetic macrocycle. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a combination therapy for the treatment of a cancer comprising a therapeutically effective amount of a p53 peptidomimetic disclosed herein or a composition comprising said p53 peptidomimetic macrocycle and a therapeutically dose of a chemotherapy agent or radiation. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

In particular embodiments, the chemotherapy agent is selected from the group consisting of actinomycin, all-trans retinoic acid, alitretinoin, azacitidine, azathioprine, bexarotene, bleomycin, bortezomib, carmofur, carboplatin, capecitabine, cisplatin, chlorambucil, cyclophosphamide, cytarabine, dacarbazine, daunorubicin, docetaxel, doxifluridine, doxorubicin, epirubicin, epothilone, etoposide, fluorouracil, gemcitabin, hydroxyurea, idarubicin, imatinib, ixabepilone, irinotecan, mechlorethamine, melphalan, mercaptopurine, methotrexate, mitoxantrone, nitrosoureas, oxaliplatin, paclitaxel, pemetrexed, romidepsin, tegafur, temozolomide (oral dacarbazine), teniposide, tioguanine, topotecan, utidelone, valrubicin, vemurafenib, vinblastine, vincristine, vindesine, vinorelbine, and vorinostat.

The present invention further provides a combination therapy for treating cancer comprising administering to a subject a therapeutically effective amount of a p53 peptidomimetic macrocycle disclosed herein or a composition comprising said p53 peptidomimetic macrocycle and a therapeutically effective amount of a checkpoint inhibitor. In particular embodiments, the checkpoint inhibitor is an anti-PD1 antibody or an anti-PD-L1 antibody. In a further embodiment, the combination therapy further includes administering to the subject a therapeutically effective dose of a chemotherapy agent or radiation. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: The present invention further provides a treatment for cancer comprising administering to a subject having the cancer a vector comprising a nucleic acid molecule encoding a wildtype p53 or p53 variant or analog with transcriptional activation activity followed by one or more administrations of a therapeutically effective amount of a p53 peptidomimetic macrocycle disclosed herein or a composition comprising said p53 peptidomimetic macrocycle.

In particular embodiments, the vector is a plasmid, a retrovirus, adenovirus, or adeno-associated virus. In particular embodiments, the subject is administered a chemotherapy or radiation treatment prior to administering the vector to the subject or subsequent to administering the vector to the subject. In further embodiments, the therapy further includes administering to the subject a checkpoint inhibitor. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

### BRIEF DESCRIPTION OF THE DRAWINGS

FIG. 1A shows a crystal structure of native human p53 peptide:MDM2 (Protein Data Bank (PDB) ID:IYCR) complex (Baek et al., JACS 134: 103-106 (2012)). MDM2 is shown as surface and bound native p53 peptide (SEQ ID NO: 15) is shown as a ribbon cartoon with interacting residues L-Phe¹⁹, L-Trp²³, and L-Leu²⁶ of the p53 peptide are highlighted in sticks. Hydrogen bond interactions are shown as dotted lines. The amino acid numbering of the p53 peptide corresponds to amino acids 15-29 of native human p53 having the amino acid sequence set forth in SEQ ID NO: 16. H96, K94, Q72, and Y100 are amino acids in the MDM2.

FIG. 1B shows a crystal structure of ^{D}PMI-δ p53 peptidomimetic peptide:MDM2 (PDB ID: 3PTX) complex (Zhan et al., J. Med. Chem. 55: 6237-6241 (2012)). MDM2 is shown as surface and bound ^{D}PMI-δ peptidomimetic macrocycle (SEQ ID NO: 11) is shown as a ribbon cartoon with interacting residues ^{D}Leu¹¹, pCF₃_^{D}Phe⁷, and 6-F-^{D}Trp³ highlighted in sticks. Hydrogen bond interactions are shown as dotted lines. H96, K94, Q72, and Y100 are amino acids in the MDM2.

FIG. 2 shows a structural representation of a representative ^{D}PMI-δ-(5-12) olefin stapled p53 peptidomimetic macrocycle: MDM2 complex taken from molecular dynamic (MD) simulations. MDM2 is shown as surface and bound ^{D}PMI-δ-(5-12) olefin stapled peptidomimetic macrocycle is shown as a stick cartoon with interacting residues highlighted in sticks. The hydrocarbon olefin linker is light portion of peptide indicated by arrow. Hydrogen bond interactions are shown as dotted lines. H96, K94, and Q72 are amino acids in the MDM2.

FIG. 3 shows the conformation of a representative ^{D}PMI-δ-(6-10) olefin stapled p53 peptidomimetic macrocycle linked to a six amino acid polypeptide tail comprising the amino acid sequence AFAAAA (SEQ ID NO: 21) in a complex with MDM2 sampled during Molecular Dynamics Simulations. MDM2 is shown as a surface/cartoon and the bound p53 peptidomimetic macrocycle is shown in cartoon with the three critical binding residues 6-F-^{D}Trp³, pCF₃_^{D}Phe⁷, and ^{D}Leu¹¹; ^{D}Phe¹⁴ in the polypeptide tail, and the olefin staple highlighted.

FIG. 4 shows the conformation of a representative ^{D}PMI-δ-(5-12) di-alkyne stapled p53 peptidomimetic macrocycle sampled during Molecular Dynamics Simulations. The p53 peptidomimetic macrocycle is shown in cartoon with the three critical binding residues and di-alkyne highlighted in sticks.

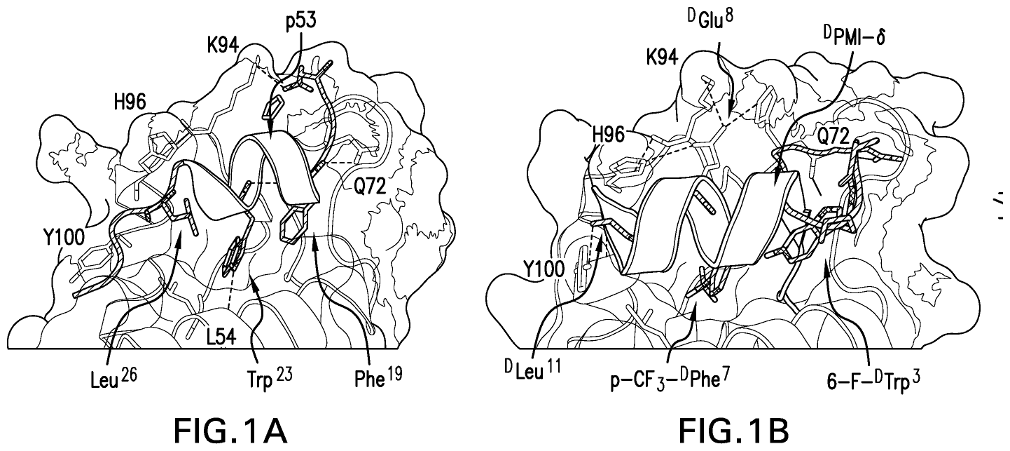

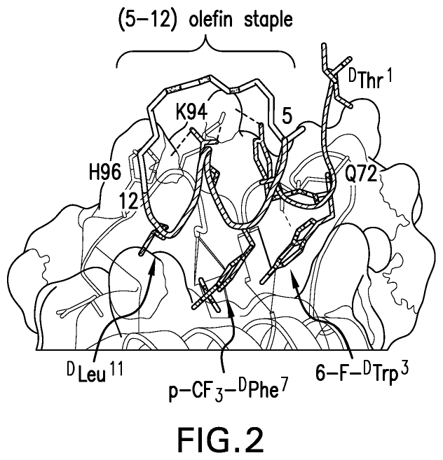

### DETAILED DESCRIPTION OF THE INVENTION

### Definitions

“Administer” and “administering” as used herein means introducing at least one p53 peptidomimetic macrocycle, or a pharmaceutical composition comprising at least one p53 peptidomimetic macrocycle, into a subject. When administration is for the purpose of treatment, the substance is provided at, or after the diagnosis of an abnormal cell growth, such as a tumor. The therapeutic administration of this substance serves to inhibit cell growth of the tumor or abnormal cell growth.

“α-amino acid” or simply “amino acid” as used herein refers to a molecule containing both an amino group and a carboxyl group bound to a carbon, which is designated the α-carbon, attached to a side chain (R group) and a hydrogen atom and may be represented by the formula shown for (R) and (S) α-amino acids

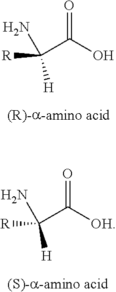

In general, L-amino acids have an (S) configuration except for cysteine, which has an (R) configuration, and glycine, which is achiral. Suitable α-amino acids for the all-D amino acid configuration peptides disclosed herein include only the D-isomers of the naturally-occurring amino acids and analogs thereof, as well as non-naturally occurring amino acids prepared by organic synthesis or other metabolic routes except for α,α-disubstituted amino acids, which may be L, D, or achiral. Unless the context specifically indicates otherwise, the term amino acid, as used herein, is intended to include amino acid analogs. As used herein, D-amino acids (or D-configuration amino acids) are denoted by the superscript “D” (e.g., ^{D}Leu) and L amino acids by “L” (e.g., L-Leu) or no L identifier (e.g., Leu). As used herein the terms D-amino acid and D-configuration amino acids are used interchangeably.

“α,α-disubstituted amino acid” as used herein refers to a molecule or moiety containing both an amino group and a carboxyl group bound to the α-carbon that is attached to two natural or non-natural amino acid side chains, or combination thereof. Exemplary α,α-disubstituted amino are shown below. These α,α-disubstituted amino acids comprise a side chain with a terminal olefinic reactive group.

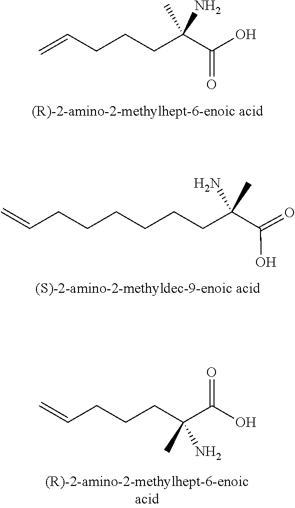

These α,α-disubstituted amino acids comprise a side chain with a terminal alkyne reactive group.

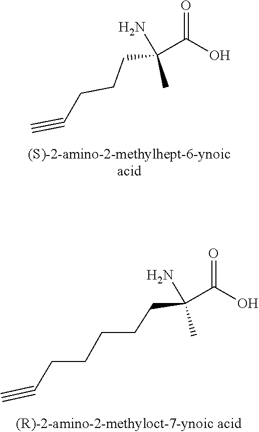

“Amino acid analog” or “non-natural amino acid” as used herein refers to a molecule which is structurally similar to an amino acid and which can be substituted for an amino acid in the formation of a p53 peptidomimetic macrocycle. Amino acid analogs include, without limitation, compounds which are structurally identical to an amino acid, as defined herein, except for the inclusion of one or more additional methylene groups between the amino and carboxyl group (e.g., α-amino, β-carboxy acids), or for the substitution of the amino or carboxy group by a similarly reactive group (e.g., substitution of the primary amine with a secondary or tertiary amine, or substitution or the carboxy group with an ester).

“Amino acid side chain” as used herein refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,α-disubstituted amino acid).

“Acyl group” as used herein refers to a moiety having the formula RCO—, wherein R is an alkane having the formula CₙH₂ₙ₊₁.

“Capping group” as used herein refers to the chemical moiety occurring at either the carboxy or amino terminus of the polypeptide chain of the subject p53 peptidomimetic macrocycle. The capping group of a carboxy terminus includes an unmodified carboxylic acid (i.e., —COOH) or a carboxylic acid with a substituent. For example, the carboxy terminus can be substituted with an amino group to yield a carboxamide at the C-terminus. Various substituents include but are not limited to primary and secondary amines, including pegylated secondary amines. The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates.

“Co-administer” as used herein means that each of at least two different biologically active compounds are administered to a subject during a time frame wherein the respective periods of biological activity overlap. Thus, the term includes sequential as well as co-extensive administration. When co-administration is used, the routes of administration need not be the same. The biological active compounds include p53 peptidomimetic macrocycles, as well as other compounds useful in treating cancer, including but not limited to agents such as vinca alkaloids, nucleic acid inhibitors, platinum agents, interleukin-2, interferons, alkylating agents, antimetabolites, corticosteroids, DNA intercalating agents, anthracyclines, and ureas. Examples of specific agents in addition to those exemplified herein, include hydroxyurea, 5-fluorouracil, anthramycin, asparaginase, bleomycin, dactinomycin, dacabazine, cytarabine, busulfan, thiotepa, lomustine, mechlorehamine, cyclophosphamide, melphalan, mechlorethamine, chlorambucil, carmustine, 6-thioguanine, methotrexate, etc. The skilled artisan will understand that two different p53 peptidomimetic macrocycles may be co-administered to a subject, or that a p53 peptidomimetic macrocycle and an agent, such as one of the agents provided above, may be co-administered to a subject.

“Combination therapy” as used herein refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

“Conservative substitution” as used herein refers to substitutions of amino acids with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see. e.g., Watson et al. Molecular Biology of the Gene, The Benjamin/Cummings Pub. Co., p. 224 (4th Ed.) (1987)). In addition, substitutions of structurally or functionally similar amino acids are less likely to disrupt biological activity. Exemplary conservative substitutions are set forth in Table 1.

TABLE 1

| Original | Conservative |
|---|---|
| residue | substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Dose”, “dosage”, “unit dose”, “unit dosage”, “effective dose” and related terms as used herein refer to physically discrete units that contain a predetermined quantity of active ingredient (e.g., p53 peptidomimetic macrocycle) calculated to produce a desired therapeutic effect (e.g., death of cancer cells). These terms are synonymous with the therapeutically-effective amounts and amounts sufficient to achieve the stated goals of the methods disclosed herein.

“^{D}PMI-δ p53 peptidomimetic peptide” as used herein refers to a linear p53 peptidomimetic peptide having the amino acid set forth in SEQ ID NO: 11 and comprising all D-configuration amino acids as indicated by the superscript “D”.

“^{D}PMI-δ (6-10) olefin stapled p53 peptidomimetic macrocycle” as used herein refers to a ^{D}PMI-δ p53 peptidomimetic peptide in which the amino acids at amino acid positions 6 and 10 are each replaced with an α,α-disubstituted amino acid comprising an amino acid sidechain having a terminal alkenyl group to provide a modified ^{D}PMI-δ p53 peptidomimetic peptide wherein macrocyclization of the modified ^{D}PMI-δ peptide through ring-closing metathesis between the two α,α-disubstituted amino acids produces the ^{D}PMI-δ (6-10) olefin stapled p53 peptidomimetic macrocycle. In specific embodiments, the other side chain on the α,α-disubstituted amino acids is a methyl substituent. ^{D}PMI-δ (6-10) olefin stapled p53 peptidomimetic macrocycles may include further amino acid modifications, insertions or substitutions, or additions to the N-terminus or C-terminus, or non-amino acid additions to the N-terminus such as acylation or to the C-terminus such as amidation.

“^{D}PMI-δ (5-12) olefin stapled p53 peptidomimetic macrocycle” as used herein refers to a ^{D}PMI-δ p53 peptidomimetic peptide in which the amino acids at amino acid positions 5 and 12 are each replaced with an α,α-disubstituted amino acid comprising an amino acid sidechain having a terminal alkenyl group to provide a modified ^{D}PMI-δ p53 peptidomimetic peptide wherein macrocyclization of the modified ^{D}PMI-δ peptide through ring-closing metathesis between the two an α,α-disubstituted amino acids produces the ^{D}PMI-δ (6-10) olefin stapled p53 peptidomimetic macrocycle. In specific embodiments, the other side chain on the α,α-disubstituted amino acids is a methyl substituent. ^{D}PMI-δ (5-12) olefin stapled p53 peptidomimetic macrocycles may include further amino acid modifications, insertions or substitutions, or additions to the N-terminus or C-terminus, or non-amino acid additions to the N-terminus such as acylation or to the C-terminus such as amidation.

“^{D}PMI-δ (5-12) di-alkyne stapled p53 peptidomimetic macrocycle” as used herein refers to a ^{D}PMI-δ p53 peptidomimetic peptide in which the amino acids at amino acid positions 5 and 12 are each replaced with an α,α-disubstituted amino acid comprising an amino acid sidechain having a terminal alkynyl group to provide a modified ^{D}PMI-δ p53 peptidomimetic peptide wherein macrocyclization of the modified ^{D}PMI-δ peptide through alkyne cross-coupling between the two an α,α-disubstituted amino acids produces the ^{D}PMI-δ (6-12) di-alkyne stapled p53 peptidomimetic macrocycle. In specific embodiments, the other side chain on the α,α-disubstituted amino acids is a methyl substituent. ^{D}PMI-δ (5-12) di-alkyne stapled p53 peptidomimetic macrocycles include further amino acid modifications, insertions or substitutions, or additions to the N-terminus or C-terminus, or non-amino acid additions to the N-terminus such as acylation or to the C-terminus such as amidation.

“Helical stability” as used herein refers to the maintenance of α-helical structure by the staples or stitch of a p53 peptidomimetic macrocycle of the invention as measured by circular dichroism or NMR. For example, in some embodiments, the p53 peptidomimetic macrocycles of the invention exhibit at least a 1.25, 1.5, 1.75 or 2-fold increase in α-helicity as determined by circular dichroism compared to a corresponding uncross-linked macrocycle.

“Macrocycle” as used herein refers to a molecule having a chemical structure including a ring or cycle formed by at least nine covalently bonded atoms.

“Macrocyclization reagent” or “macrocycle-forming reagent” as used herein refers to any reagent which may be used to prepare a p53 peptidomimetic macrocycle of the invention by mediating the reaction between two reactive groups on the α,α-disubstituted amino acids. Reactive groups on the α,α-disubstituted amino acids may be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate.

Macrocyclization reagents may additionally include, for example, Ru reagents known in the art such as Cp*RuCl(PPh₃)₂, [Cp*RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Acc. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

“p53” as used herein refers to tumor protein P53, also known as cellular tumor antigen p53 (UniProt name), phosphoprotein p53, tumor suppressor p53, antigen NY—CO-13, or transformation-related protein 53 (TRP53), or any isoform of a protein encoded by homologous genes in various organisms, for example, the TP53 gene in humans.

“MDM2” as used herein refers to the mouse double minute 2 protein also known as E3 ubiquitin-protein ligase. MDM2 is a protein that in humans is encoded by the MDM2 gene. MDM2 protein is an important negative regulator of the p53 tumor suppressor. MDM2 protein functions both as an E3 ubiquitin ligase that recognizes the N-terminal transactivation domain (TAD) of the p53 tumor suppressor and as an inhibitor of p53 transcriptional activation. As used herein, the term MDM2 refers to the human homolog. See GenBank Accession No.: 228952; GI:228952.

“MDMX” or “MDM4” as used herein refers to mouse double minute X or 4, a protein that shows significant structural similarity to MDM2. MDMX or MDM4 interacts with p53 via a binding domain located in the N-terminal region of the MDMX or MDM4 protein. As used herein, the term MDMX or MDM4 refers to the same human homolog. See GenBank Accession No.: 88702791; GI:88702791.

“Transcription activation activity” as used herein with respect to p53 or variant or analog thereof refers to the ability of the p53 or variant or analog thereof to activate transcription from a p53-dependent promoter. The activation ability of p53 or variant or analog thereof may be determined in a transcription assay that enables expression of a reporter gene operably linked to p53-dependent promoter to be detected and measured.

“Member” as used herein in conjunction with macrocycles or macrocycle-forming linkers refers to the atoms that form or can form the macrocycle and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

“Naturally occurring amino acid” or “natural amino acid” as used herein refers to any one of the twenty amino acids commonly found in peptides synthesized in nature, and known by the one letter abbreviations A, R, N, C, D, Q, E, G, H, I, L, K, M, F, P, S, T, W, Y and V, naturally occurring amino acids may have a D-configuration (D-amino acid) or an L-configuration (L-amino acid).

“Non-naturally occurring amino acid” or “non-natural amino acid” as used herein refers to an amino acid analog that is not found in nature.

“Non-essential” amino acid residue is a residue that can be altered from the wild-type sequence of a polypeptide without abolishing or substantially altering the polypeptide's essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

“Peptidomimetic macrocycle” or “crosslinked polypeptide” as used herein refers to a compound comprising a plurality of amino acid residues joined by a plurality of peptide bonds and at least one macrocycle-forming linker, which forms a macrocycle between a first naturally occurring or non-naturally occurring amino acid residue (or analog) and a second naturally occurring or non-naturally occurring amino acid residue (or analog) within the same molecule. The peptidomimetic macrocycle include embodiments where the macrocycle-forming linker connects the α-carbon of the first α,α-disubstituted amino acid residue (or analog) to the α-carbon of the second α,α-disubstituted amino acid residue (or analog). Peptidomimetic macrocycles optionally include one or more non-peptide bonds between one or more amino acid residues and/or amino acid analog residues, and optionally include one or more non-naturally occurring amino acid residues or amino acid analog residues in addition to any which form the macrocycle. A “corresponding non-crosslinked polypeptide” when referred to in the context of a peptidomimetic macrocycle is understood to relate to a polypeptide of the same amino acid sequence as the peptidomimetic macrocycle except for those amino acids involved in the staple or stitch crosslinks.

“Di-alkyne” as used herein refers two alkynes separated by a single bond, for example as represented by the structure

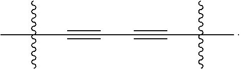 [SMILES: *C#CC#C*]

Unless otherwise stated, compounds and structures referred to herein are also meant to include compounds which differ only in the presence of one or more isotopically enriched atoms. For example, compounds having the present structures wherein hydrogen is replaced by deuterium or tritium, or wherein carbon atom is replaced by ¹³C- or ¹⁴C-enriched carbon, or wherein a carbon atom is replaced by silicon, are within the scope of this invention. The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

“Pharmaceutically acceptable derivative” as used herein means any pharmaceutically acceptable salt, ester, salt of an ester, pro-drug or other derivative of a p⁵³ peptidomimetic macrocycle disclosed herein, which upon administration to an individual, is capable of providing (directly or indirectly) a p53 peptidomimetic macrocycle disclosed herein. Particularly favored pharmaceutically acceptable derivatives are those that increase the bioavailability of the p53 peptidomimetic macrocycle disclosed herein when administered to an individual (e.g., by increasing absorption into the blood of an orally administered p53 peptidomimetic macrocycle disclosed herein) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

“Polypeptide” as used herein encompasses two or more naturally or non-naturally occurring amino acids joined by a covalent bond (e.g., an amide bond). Polypeptides as described herein include full length proteins (e.g., fully processed proteins) as well as shorter amino acid sequences (e.g., fragments of naturally occurring proteins or synthetic polypeptide fragments).

“Stability” as used herein refers to the maintenance of a defined secondary structure in solution by a p53 peptidomimetic macrocycle of the invention as measured by circular dichroism, NMR or another biophysical measure, or resistance to proteolytic degradation in vitro or in vivo. Non-limiting examples of secondary structures contemplated in this invention are α-helices, β-turns, and β-pleated sheets.

“Therapeutically effective amount” or “therapeutically effective dose” as used herein refers to a quantity of a specific substance sufficient to achieve a desired effect in a subject being treated. For instance, this may be the amount of p53 peptidomimetic macrocycle of the present invention necessary to activate p53 by inhibiting its binding to MDM2 and MDMX. It may also refer to the amount or dose of a chemotherapy agent or radiation administered to a subject that has cancer that is commonly administered to the subject to treat the cancer.

“Treat” or “treating” as used herein means to administer a therapeutic agent, such as a composition containing any of the p53 peptidomimetic macrocycles of the present invention, internally or externally to a subject or patient having one or more disease symptoms, or being suspected of having a disease, for which the agent has therapeutic activity or prophylactic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a human or animal subject with a disorder, disease or symptom, or with the potential to develop such a disorder, disease or symptom.

“Treatment” as it applies to a human or veterinary individual, as used herein refers to therapeutic treatment, which encompasses contact of a p53 peptidomimetic macrocycle of the present invention to a human or animal individual who is in need of treatment with the p53 peptidomimetic macrocycle of the present invention where the p53 peptidomimetic macrocycle has therapeutic activity or prophylactic activity.

### P53 Peptidomimetic Macrocycles

Peptide-based inhibitors hold great potential for targeted modulation of intracellular protein-protein interactions (PPIs) since they can access a huge range of chemical space including sequence diversity as well as a host of different secondary and tertiary structures. Currently, liabilities that hinder the development of peptide therapeutics include poor conformational stability, proteolytic sensitivity, and cell permeability. In addition, translation of these in vitro peptide inhibitors into intracellularly active and in vivo active compounds with on target selectivity and specificity is difficult. Several contemporary peptide design strategies address these issues to different degrees. Strategic macrocyclization through optimally placed chemical braces such as olefin hydrocarbon crosslinks, commonly referred to as staples, can address these issues by (i) restricting conformational freedom to give improved target affinities, (ii) improving proteolytic resistance, and (iii) enhancing cell permeability. Conversely, molecules constructed entirely from D-amino acids are hyper-resistant to proteolytic cleavage but generally lack conformational stability and membrane permeability. We have discovered that all-D α-helical stapled peptides (hereinafter referred to as peptide macrocycles) can be designed by using particular staples and adding three to nine amino acid polypeptide tails to the C-terminus of the peptide macrocycle exhibit improved pharmacological properties. These improvements in pharmacological properties include protease resistance, conformational stability, cell permeability without induction of membrane disruption, and low or no cellular toxicity (e.g., the p53 peptidomimetic peptides display reduced cellular activity with counterscreen activity and LDH release—determined using assays disclosed below in General Methods). Furthermore, the p53 peptidomimetic macrocycles of the present invention bind mouse double minute 2 (MDM2 aka E3 ubiquitin-protein ligase) and MDMX (aka MDM4) and intracellularly activate p53 by binding MDM2 and MDMX, thereby antagonizing MDM2 and MDMX binding to p53.

We have exemplified our discovery by adding a polypeptide tail comprising three to nine amino acids to the C-terminal amino acid of an all-D p53 peptidomimetic macrocycle that had been derived from the linear ^{D}PMI-δ peptide (disclosed in Liu et al. [41] and in U.S. Pub. Patent No. 20120328692; SEQ ID NO: 11) and comprising an olefin staple between the α-carbons of two α,α-disubstituted amino acids located at amino acid positions 6 and 10 of the peptide (6-10 olefin staple) as disclosed in WO2020257153 to produce a p53 peptidomimetic macrocycle having the improved pharmacological properties. Further improvements may be achieved by replacing the amino acid at position 9 with glutamine and the amino acid at position 12 with serine.

We also discovered that improvements to the pharmacological properties of a ^{D}PMI-δ peptide comprising an olefin staple between amino acid positions 5 and 12 of the peptide (5-12 olefin staple) could be achieved by replacing the 5-12 olefin stable with a 5-12 di-alkyne staple and optionally adding a three to nine amino acid polypeptide tail to the C-terminal. A further improvement may be achieved by replacing the amino acid at position 9 with glutamine.

The present invention provides a p53 peptidomimetic macrocycle, comprising (a) an i, i+4 olefin staple and a polypeptide tail covalently linked at its N-terminus to the C-terminal amino acid of the p53 peptidomimetic macrocycle; (b) an i, i+7 olefin staple and a polypeptide tail covalently linked at its N-terminus to the C-terminal amino acid of the p53 peptidomimetic macrocycle; or (c) an i, i+7 di-alkyne staple and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

In a further embodiment of the p53 peptidomimetic macrocycle, the p53 peptidomimetic macrocycle comprises an i, i+7 alkyne staple and is covalently linked at the C-terminal amino acid to the N-terminus of the polypeptide tail.

In a further embodiment of the p53 peptidomimetic macrocycle, the p53 peptidomimetic macrocycle comprises 12 amino acids and an i, i+4 olefin staple formed between the α-carbons of two α,α-disubstituted amino acids located at amino acid positions 6 and 10 of the p53 peptidomimetic macrocycle and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

In a further embodiment of the p53 peptidomimetic macrocycle, the α,α-disubstituted amino acids at amino acid positions 6 and 10 of the p53 peptidomimetic macrocycle comprise (R)-2-amino-2-methylhept-6-enoic acid.

In a further embodiment of the p53 peptidomimetic macrocycle, the p53 peptidomimetic macrocycle further comprises D-6-fluoro-tryptophane at amino acid position 3 and D-p-CF3-phenylalanine at amino acid position 7.

In a further embodiment of the p53 peptidomimetic macrocycle, the p53 peptidomimetic macrocycle further comprises threonine at amino acid position 1, alanine at amino acid position 2, tyrosine at amino acid position 4, alanine at amino acid position 5, glutamic acid at amino acid position 8, lysine or glutamine at amino acid position 9, leucine at amino acid position 11, and arginine or serine at amino acid position 12.

In a further embodiment of the p53 peptidomimetic macrocycle, the p53 peptidomimetic macrocycle comprises glutamine at amino acid at position 9 and serine at amino acid at position 12.

In a further embodiment of the p53 peptidomimetic macrocycle, the p53 peptidomimetic macrocycle comprises 12 amino acids and an i, i+7 a di-alkyne staple formed between the α-carbons of two α,α-disubstituted amino acids located at amino acid positions 5 and 12 of the p53 peptidomimetic macrocycle, and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration.

In a further embodiment of the p53 peptidomimetic macrocycle, the α,α-disubstituted amino acid at amino acid position 5 of the p53 peptidomimetic macrocycle comprises (S)-2-amino-2-methylhept-6-ynoic acid and the α,α-disubstituted amino acid at amino acid position 12 of the p53 peptidomimetic macrocycle comprises (R)-2-amino-2-methyloct-7-ynoic acid.

In a further embodiment of the p53 peptidomimetic macrocycle, the p53 peptidomimetic macrocycle further comprises D-6-fluoro-tryptophane at amino acid position 3 of the p53 peptidomimetic macrocycle and D-p-CF3-phenylalanine at amino acid position 7 of the p53 peptidomimetic macrocycle.

In a further embodiment of the p53 peptidomimetic macrocycle, the p53 peptidomimetic macrocycle further comprises threonine at amino acid at position 1, alanine at amino acid position 2, tyrosine at amino acid position 4, asparagine at amino acid position 6, glutamic acid at amino acid position 8, lysine or glutamine at amino acid position 9, leucine at amino acid position 10, and leucine at amino acid position 11.

In a further embodiment of the p53 peptidomimetic macrocycle, the p53 peptidomimetic macrocycle comprises glutamine at amino acid position 9.

In a further embodiment of the p53 peptidomimetic macrocycle, the p53 peptidomimetic macrocycle comprises 12 amino acids and an i, i+7 olefin staple formed between the α-carbons of two α,α-disubstituted amino acids located at amino acid positions 5 and 12 of the p53 peptidomimetic macrocycle, and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration.

In a further embodiment of the p53 peptidomimetic macrocycle, the α,α-disubstituted amino acid at amino acid position 5 of the p53 peptidomimetic macrocycle comprises (S)-2-amino-2-methyldec-9-enoic acid and the α,α-disubstituted amino acid at amino acid position 12 of the p53 peptidomimetic macrocycle comprises (R)-2-amino-2-methylhept-6-enoic acid.

In a further embodiment of the p53 peptidomimetic macrocycle, the p53 peptidomimetic macrocycle further comprises D-6-fluoro-tryptophane at amino acid position 3 of the p53 peptidomimetic macrocycle and D-p-CF3-phenylalanine at amino acid position 7 of the p53 peptidomimetic macrocycle.

In a further embodiment of the p53 peptidomimetic macrocycle, the p53 peptidomimetic macrocycle further comprises threonine at amino acid at position 1, alanine at amino acid position 2, tyrosine at amino acid position 4, asparagine at amino acid position 6, glutamic acid at amino acid position 8, lysine or glutamine at amino acid position 9, leucine at amino acid position 10, and leucine at amino acid position 11.

In a further embodiment of the p53 peptidomimetic macrocycle, the p53 peptidomimetic macrocycle comprises glutamine at amino acid position 9.

In a further embodiment of the p53 peptidomimetic macrocycle, the polypeptide tail comprises three to nine amino acids, each amino acid of the polypeptide tail independently having a D-configuration or an L-configuration or, in specific embodiments, each amino acid in the polypeptide tail having a D-configuration.

In a further embodiment of the p53 peptidomimetic macrocycle, the polypeptide tail comprises six amino acids, each amino acid of the polypeptide tail independently having a D-configuration or an L-configuration or, in specific embodiments, each amino acid in the polypeptide tail having a D-configuration.

In a further embodiment of the disclosed p53 peptidomimetic macrocycles, the polypeptide tail comprises an amino acid sequence set forth in SEQ ID NO: 20, SEQ ID NO: 21, SEQ ID NO: 22, or SEQ ID NO: 23.

The present invention further provides a p53 peptidomimetic macrocycle comprising:

(SEQ ID NO: 17)
TAX³YAX⁶X⁷EKX¹⁰X¹¹X¹²X¹³X¹⁴X¹⁵X¹⁶X¹⁷X¹⁸-X¹⁹X²⁰X²¹

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁶ is (R)-2-amino-2-methylhept-6-enoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X¹⁰ is (R)-2-amino-2-methylhept-6-enoic acid; X¹¹ is D-Leu; X¹² is D-Arg or D-Ser; X¹³ is D-Ala, D-Glu, or D-Gla (γ-carboxylic glutamic acid); X¹⁴ is D-Ala; X¹⁵ is D-Ala, D-Phe, or D-Glu; X¹⁶ is D-Ala or absent; X¹⁷ is D-Ala, D-α-methyl-Glu, or absent; X¹⁸ is D-Ala or absent; X¹⁹ is an D-Ala or absent; X²⁰ is D-Ala or absent; X²¹ is D-Ala or absent; the N-terminal amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; and the staple is an olefin obtained through ring-closing metathesis between X⁶ and X¹⁰.

In particular embodiments, amino acids 1-12 are all D amino acids and amino acids 13-21 are each independently an L-amino acid or D-amino acid or are each a D-amino acid.

In a further embodiment of the p53 peptidomimetic macrocycle, X¹² is D-Ser. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

The present invention provides a p53 peptidomimetic macrocycle comprising:

(SEQ ID NO: 18)
TAX³YX⁵NX⁷EX⁹LX¹¹X¹²X¹³X¹⁴X¹⁵X¹⁶X¹⁷X¹⁸-X¹⁹X²⁰X²¹

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁵ is (S)-2-amino-2-methylhept-6-ynoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X⁹ is D-Lys or D-Gln; X¹¹ is D-Leu; X¹² is (R)-2-amino-2-methyloct-7-ynoic acid; X¹³ is D-Ala, D-Glu, D-Gla (γ-carboxylic glutamic acid) or absent; X¹⁴ is D-Ala or absent; X⁵ is D-Ala, D-Phe, or D-Glu or absent; X¹⁶ is D-Ala or absent; X¹⁷ is D-Ala, D-α-methyl-Glu, or absent; X¹⁸ is D-Ala or absent; X¹⁹ is D-Ala or absent; X²⁰ is D-Ala or absent; X²¹ is D-Ala or absent; the N-terminus amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; and the staple is a di-alkyne obtained through alkyne cross-coupling between X⁵ and X¹².

In particular embodiments, amino acids 1-12 are all D amino acids and amino acids 13-21 are each independently an L-amino acid or D-amino acid or are each a D-amino acid.

In a further embodiment of the p53 peptidomimetic macrocycle, X⁹ is D-Gln. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

The present invention provides a p53 peptidomimetic macrocycle comprising:

(SEQ ID NO: 27)
TAX³YX⁵NX⁷EX⁹LX¹¹X¹²X¹³X¹⁴X¹⁵X¹⁶X¹⁷X¹⁸X¹⁹X²⁰X²¹

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁵ is (S)-2-amino-2-methyldec-9-enoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X⁹ is D-Lys or D-Gln; X¹¹ is D-Leu; X¹² is (R)-2-amino-2-methylhept-6-enoic acid; X¹³ is D-Ala, D-Glu, D-Gla (γ-carboxylic glutamic acid); X₁₄ is D-Ala; X¹⁵ is D-Ala, D-Phe, or D-Glu; X¹⁶ is D-Ala; X¹⁷ is D-Ala or D-α-methyl-Glu; X¹⁸ is D-Ala; X¹⁹ is D-Ala or absent; X²⁰ is D-Ala or absent; X²¹ is D-Ala or absent; the N-terminus amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; and the staple is an olefin obtained through ring-closing metathesis between X⁵ and X².

In particular embodiments, amino acids 1-12 are all D amino acids and amino acids 13-21 are each independently an L-amino acid or D-amino acid or are each a D-amino acid.

In a further embodiment of the p53 peptidomimetic macrocycle, X⁹ is D-Gln. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

The present invention provides a p53 peptidomimetic macrocycle comprising:

(SEQ ID NO: 19)
TAX³YX⁵NX⁷EX⁹LX¹¹X¹²

wherein X³ is D-Trp, D-6-Fluoro-Trp, D-6-Chloro-Trp, D-6-Bromo-Trp, D-6-Iodo-Trp, D-6-methyl-Trp, D-6-cyano-Trp, D-6-hydroxy-Trp, D-6-NO₂-Trp, D-7-Fluoro-Trp, D-7-Chloro-Trp, D-7-Bromo-Trp, D-7-Iodo-Trp, D-7-methyl-Trp, D-7-cyano-Trp, D-7-hydroxy-Trp, D-7-NO₂-Trp, D-6,7-Fluoro-Trp, D-6,7-Chloro-Trp, D-6,7-Bromo-Trp, D-6,7-Iodo-Trp, D-6,7-methyl-Trp, D-6,7-cyano-Trp, D-6,7-hydroxy-Trp, or D-6,7-NO₂-Trp; X⁵ is (S)-2-amino-2-methylhept-6-ynoic acid; X⁷ is D-p-Fluoro-Phe, D-p-Chloro-Phe, D-p-Bromo-Phe, D-p-Iodo-Phe, D-p-methyl-Phe, D-p-cyano-Phe, D-p-hydroxy-Phe, D-p-NO₂—Phe, or D-p-CF₃—Phe; X⁹ is D-Lys or D-Gln; X₁₁ is D-Leu; X¹² is (R)-2-amino-2-methyloct-7-ynoic acid; the N-terminus amino group of the Thr at position 1 is conjugated to an acyl group having the formula RCO—; R is an alkane having the formula CₙH₂ₙ₊₁; n is an integer from 1 to 10; the C-terminal amino acid optionally comprises an amino group; amino acids 1-12 are D amino acids; and, the staple is a di-alkyne obtained through alkyne cross-coupling between X⁵ and X¹².

In a further embodiment of the p53 peptidomimetic macrocycle, X⁹ is D-Gln. In a further embodiment of the p53 peptidomimetic macrocycle, X³ is D-6-Fluoro-Trp; X⁷ is D-p-CF₃—Phe; or X³ is D-6-Fluoro-Trp and X⁷ is D-p-CF₃—Phe. In a further embodiment, the acyl group is an acetyl group.

In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a p53 peptidomimetic macrocycle having a structure

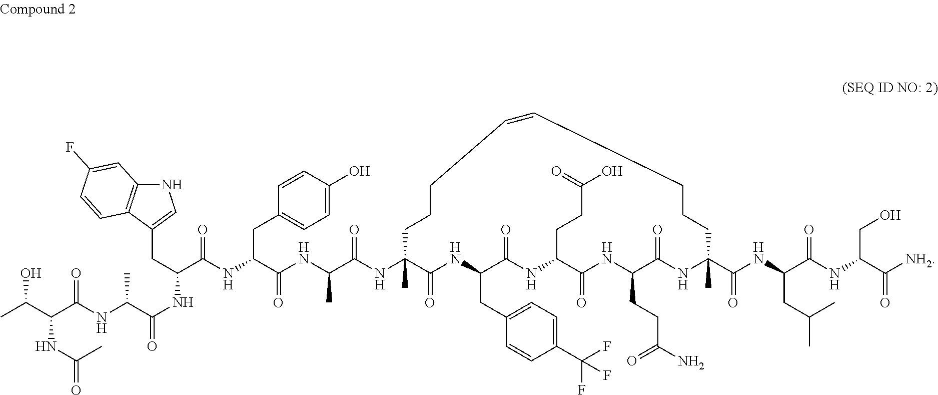

The present invention further provides a p53 peptidomimetic macrocycle having a structure

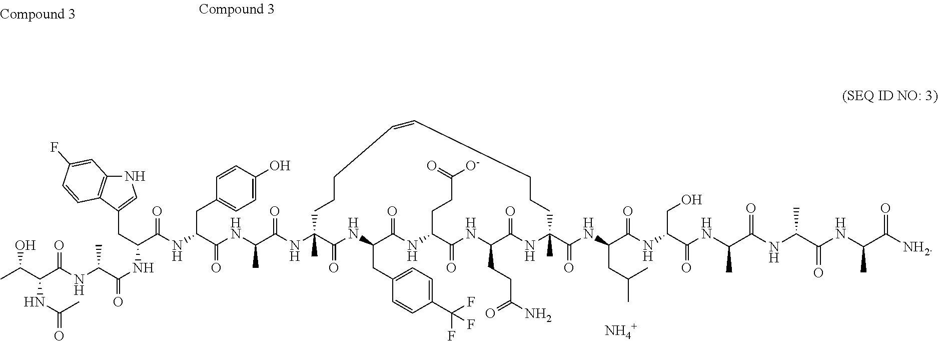

The present invention further provides a p53 peptidomimetic macrocycle having a structure

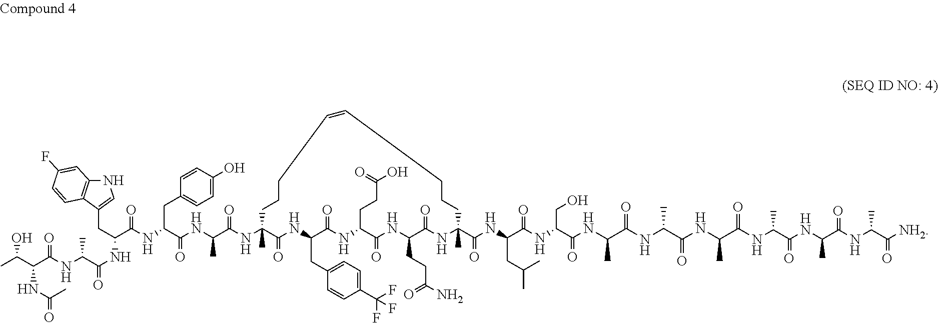

The present invention further provides a p53 peptidomimetic macrocycle having a structure

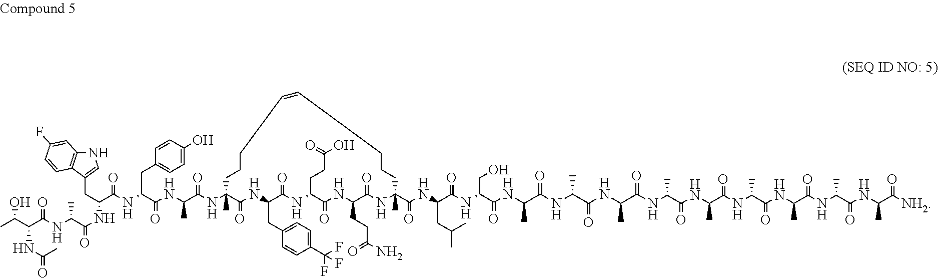

The present invention further provides a p53 peptidomimetic macrocycle having a structure

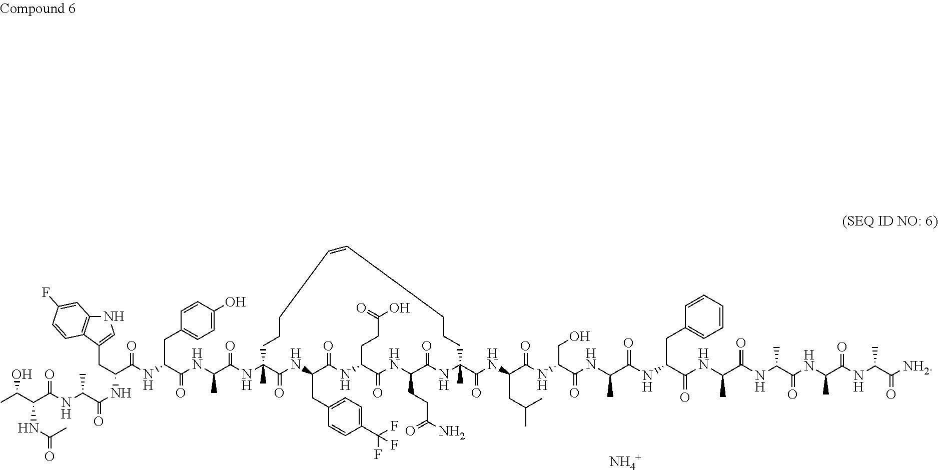

The present invention further provides a p53 peptidomimetic macrocycle having a structure

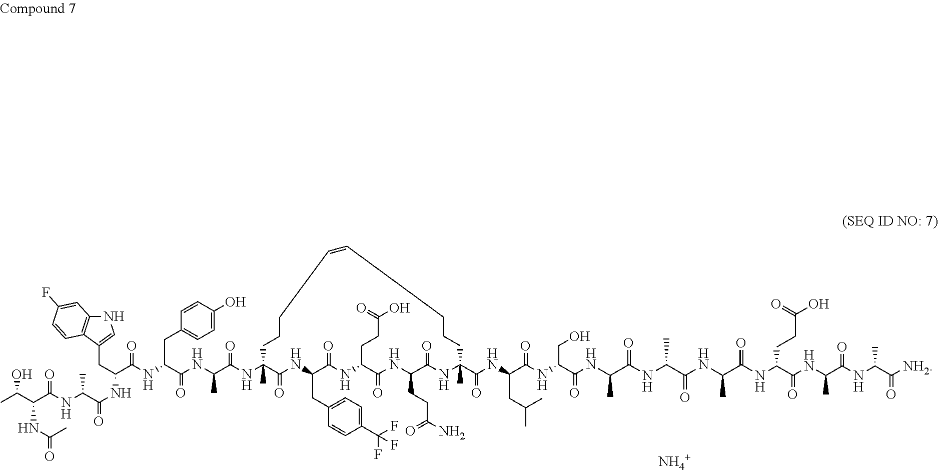

The present invention further provides a p53 peptidomimetic macrocycle having a structure

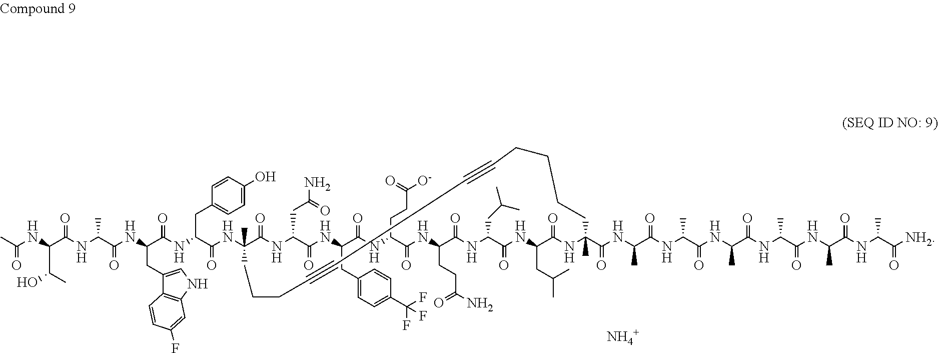

The present invention further provides a p53 peptidomimetic macrocycle having a structure

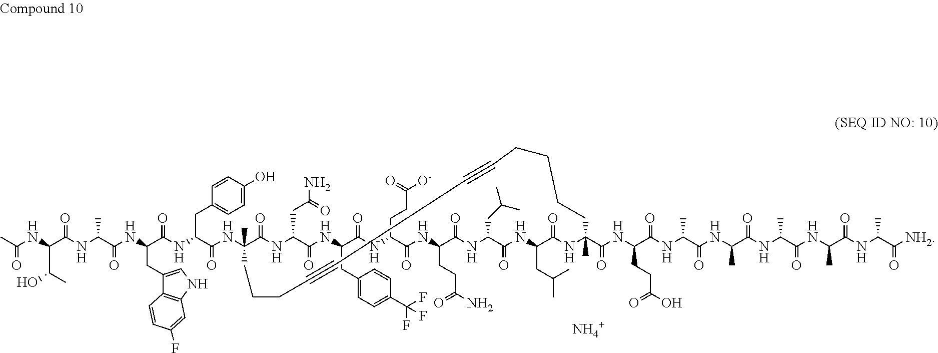

The present invention further provides a p53 peptidomimetic macrocycle having a structure

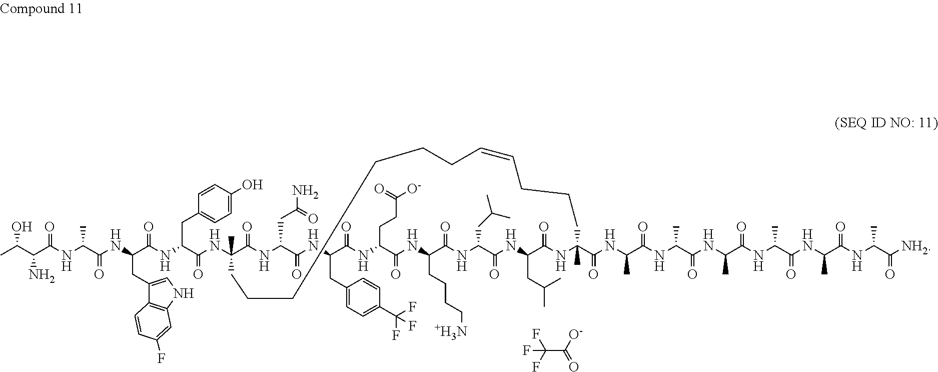

The present invention further provides a composition comprising a p53 peptidomimetic macrocycle disclosed herein and a pharmaceutically acceptable carrier. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a method for treating cancer in a subject in need thereof comprising administering to the subject a p53 peptidomimetic macrocycle disclosed herein or a composition comprising said p53 peptidomimetic macrocycle. The present invention further provides the use of a peptidomimetic macrocycle disclosed herein for the preparation of a medicament for treating cancer. The present invention further provides a peptidomimetic macrocycle disclosed herein for the treatment of cancer. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

In a further embodiments, the cancer is selected from the group consisting of melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, salivary cancer, pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, and cancer of hematological tissues.

The present invention further provides a method of modulating the activity of p53 and/or MDM2 and/or MDMX in a subject comprising administering to the subject a p53 peptidomimetic macrocycle disclosed herein, or a composition comprising said p53 peptidomimetic macrocycle. The present invention further provides the use of a p53 peptidomimetic macrocycle disclosed herein for the preparation of a medicament for modulating said activity. The present invention further provides a p53 peptidomimetic macrocycle disclosed herein for the modulation of said activity. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a method of antagonizing the interaction between p53 and MDM2 and/or between p53 and MDMX in a subject comprising administering to the subject a p53 peptidomimetic macrocycle disclosed herein, or a composition comprising said p53 peptidomimetic macrocycle. The present invention further provides the use of a p53 peptidomimetic macrocycle disclosed herein for the preparation of a medicament for antagonizing the interaction between p53 and MDM2 and/or between p53 and MDMX. The present invention further provides a p53 peptidomimetic macrocycle disclosed herein for antagonizing the interaction between p53 and MDM2 and/or between p53 and MDMX. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a combination therapy for treating cancer comprising administering to a subject a therapeutically effective amount of a p53 peptidomimetic macrocycle disclosed herein or a composition comprising said p53 peptidomimetic macrocycle and a therapeutically effective dose of a chemotherapy agent or radiation. In a further embodiment, the chemotherapy agent or radiation is administered to the subject followed by administration of the p53 peptidomimetic macrocycle; the p53 peptidomimetic macrocycle is administered to the subject followed by administration of the chemotherapy agent or radiation; or the chemotherapy agent or radiation is administered to the subject simultaneously with administration of the p53 peptidomimetic macrocycle. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a combination therapy for the treatment of a cancer comprising a therapeutically effective amount of a p53 peptidomimetic macrocycle disclosed herein or a composition comprising said p53 peptidomimetic macrocycle and a therapeutically dose of a chemotherapy agent or radiation. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

In particular embodiments, the chemotherapy agent is selected from the group consisting of actinomycin, all-trans retinoic acid, alitretinoin, azacitidine, azathioprine, bexarotene, bleomycin, bortezomib, carmofur, carboplatin, capecitabine, cisplatin, chlorambucil, cyclophosphamide, cytarabine, dacarbazine, daunorubicin, docetaxel, doxifluridine, doxorubicin, epirubicin, epothilone, etoposide, fluorouracil, gemcitabin, hydroxyurea, idarubicin, imatinib, ixabepilone, irinotecan, mechlorethamine, melphalan, mercaptopurine, methotrexate, mitoxantrone, nitrosoureas, oxaliplatin, paclitaxel, pemetrexed, romidepsin, tegafur, temozolomide (oral dacarbazine), teniposide, tioguanine, topotecan, utidelone, valrubicin, vemurafenib, vinblastine, vincristine, vindesine, vinorelbine, and vorinostat.

The present invention further provides a combination therapy for treating cancer comprising administering to a subject in need thereof a therapeutically effective amount of a p53 peptidomimetic macrocycle disclosed herein or a composition comprising said p53 peptidomimetic macrocycle and a therapeutically effective amount of a checkpoint inhibitor. In particular embodiments, the checkpoint inhibitor is an anti-PD1 antibody or an anti-PD-L1 antibody. In a further embodiment, the combination therapy further includes administering to the subject a therapeutically effective dose of a chemotherapy agent or radiation. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

The present invention further provides a treatment for cancer comprising administering to a subject having the cancer a vector comprising a nucleic acid molecule encoding a wild-type p53 or p53 variant or analog with transcriptional activation activity followed by one or more administrations of a therapeutically effective amount of a p53 peptidomimetic macrocycle disclosed herein or a composition comprising said p53 peptidomimetic macrocycle. In particular embodiments, the vector is a plasmid, a retrovirus, adenovirus, or adeno-associated virus. In particular embodiments, the subject is administered a chemotherapy or radiation treatment prior to administering the vector to the subject or subsequent to administering the vector to the subject. In further embodiments, the therapy further includes administering to the subject a checkpoint inhibitor. In a further embodiment, the p53 peptidomimetic macrocycle comprises an amino acid sequence set forth in SEQ ID NO: 2, SEQ ID NO: 3, SEQ ID NO: 4, SEQ ID NO: 5, SEQ ID NO: 6, SEQ ID NO: 7, SEQ ID NO: 9, or SEQ ID NO: 10. In a further embodiment, the p53 peptidomimetic macrocycle comprises an amino acid sequence set forth in SEQ ID NO: 4, SEQ ID NO: 6, SEQ ID NO: 7, or SEQ ID NO: 9.

### Pharmaceutical Compositions

The present invention also provides pharmaceutical compositions comprising a p53 peptidomimetic macrocycle of the present invention. The p53 peptidomimetic macrocycle may be used in combination with any suitable pharmaceutical carrier or excipient. Such pharmaceutical compositions comprise a therapeutically effective amount of one or more p53 peptidomimetic macrocycles, and pharmaceutically acceptable excipient(s) and/or carrier(s). The specific formulation will suit the mode of administration. In particular aspects, the pharmaceutical acceptable carrier may be water or a buffered solution.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, administration aids, and combinations thereof.

Carriers are compounds and substances that improve and/or prolong the delivery of an active ingredient to a subject in the context of a pharmaceutical composition. Carrier may serve to prolong the in vivo activity of a drug or slow the release of the drug in a subject, using controlled-release technologies. Carriers may also decrease drug metabolism in a subject and/or reduce the toxicity of the drug. Carrier can also be used to target the delivery of the drug to particular cells or tissues in a subject. Common carriers (both hydrophilic and hydrophobic carriers) include fat emulsions, lipids, PEGylated phospholipids, PEGylated liposomes, PEGylated liposomes coated via a PEG spacer with a cyclic RGD peptide c(RGD^{DYK}), liposomes and lipospheres, microspheres (including those made of biodegradable polymers or albumin), polymer matrices, biocompatible polymers, protein-DNA complexes, protein conjugates, erythrocytes, vesicles, nanoparticles, and side-chains for hydro-carbon stapling. The aforementioned carriers can also be used to increase cell membrane permeability of the p53 peptidomimetic macrocycles of the invention. In addition to their use in the pharmaceutical compositions of the present invention, carriers may also be used in compositions for other uses, such as research uses in vitro (e.g., for delivery to cultured cells) and/or in vivo.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils, e.g. vegetable oils, may be used to provide oil-in-water or water in oil suspensions. In certain situations, delayed release preparations may be advantageous and compositions which can deliver the p53 peptidomimetic macrocycles in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers, or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain antioxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerin and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules and tablets. The pharmaceutical compositions may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants. They may also contain therapeutically active agents in addition to the substance of the present invention.

The pharmaceutical compositions may be administered in a convenient manner such as by the topical, intravenous, intraperitoneal, intramuscular, intratumor, subcutaneous, intranasal, or intradermal routes. The pharmaceutical compositions are administered in an amount which is effective for treating and/or prophylaxis of the specific indication. In general, the pharmaceutical compositions are administered in an amount of at least about 0.1 mg/kg to about 100 mg/kg body weight. In most cases, the dosage is from about 10 mg/kg to about 1 mg/kg body weight daily, taking into account the route of administration, symptoms, etc.

Dosages of the p53 peptidomimetic macrocycles of the present invention can vary between wide limits, depending upon the location, source, identity, extent and severity of the cancer, the age and condition of the individual to be treated, etc. A physician will ultimately determine appropriate dosages to be used.

The p53 peptidomimetic macrocycles may also be employed in accordance with the present invention by expression of the antagonists in vivo, i.e., via gene therapy. The use of the peptides or compositions in a gene therapy setting is also considered to be a type of “administration” of the peptides for the purposes of the present invention.

Accordingly, the present invention also relates to methods of treating a subject having cancer, comprising administering to the subject a pharmaceutically effective amount of one or more p53 peptidomimetic macrocycle of the present invention, or a pharmaceutical composition comprising one or more of the antagonists to a subject needing treatment. The term “cancer” is intended to be broadly interpreted and it encompasses all aspects of abnormal cell growth and/or cell division. Examples include: carcinoma, including but not limited to adenocarcinoma, squamous cell carcinoma, adenosquamous carcinoma, anaplastic carcinoma, large cell carcinoma, small cell carcinoma, and cancer of the skin, breast, prostate, bladder, vagina, cervix, uterus, liver, kidney, pancreas, spleen, lung, trachea, bronchi, colon, small intestine, stomach, esophagus, gall bladder; sarcoma, including but not limited to chondrosarcoma, Ewing's sarcoma, malignant hemangioendothelioma, malignant schwannoma, osteosarcoma, soft tissue sarcoma, and cancers of bone, cartilage, fat, muscle, vascular, and hematopoietic tissues; lymphoma and leukemia, including but not limited to mature B cell neoplasms, such as chronic lymphocytic leukemia/small lymphocytic lymphoma, B-cell prolymphocytic leukemia, lymphomas, and plasma cell neoplasms, mature T cell and natural killer (NK) cell neoplasms, such as T cell prolymphocytic leukemia, T cell large granular lymphocytic leukemia, aggressive NK cell leukemia, and adult T cell leukemia/lymphoma, Hodgkin lymphomas, and immunodeficiency-associated lymphoproliferative disorders; germ cell tumors, including but not limited to testicular and ovarian cancer; blastoma, including but not limited to hepatoblastoma, medulloblastoma, nephroblastoma, neuroblastoma, pancreatoblastoma, leuropulmonary blastoma and retinoblastoma. The term also encompasses benign tumors.

In each of the embodiments of the present invention, the individual or subject receiving treatment is a human or non-human animal, e.g., a non-human primate, bird, horse, cow, goat, sheep, a companion animal, such as a dog, cat or rodent, or other mammal. In some embodiments, the subject is a human.

The invention also provides a kit comprising one or more containers filled with one or more of the ingredients of the pharmaceutical compositions of the invention, such as a container filled with a pharmaceutical composition comprising a p53 peptidomimetic macrocycle of the present invention and a pharmaceutically acceptable carrier or diluent. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions may be employed in conjunction with other therapeutic compounds.

### Combination Therapy Comprising Chemotherapy

The p53 peptidomimetic macrocycle of the present invention may be administered to an individual having a cancer in combination with chemotherapy. The individual may undergo the chemotherapy at the same time the individual is administered the p53 peptidomimetic macrocycle. The individual may undergo chemotherapy after the individual has completed a course of treatment with the p53 peptidomimetic macrocycle. The individual may be administered the p53 peptidomimetic macrocycle after the individual has completed a course of treatment with a chemotherapy agent. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase I, including but not limited to, irinotecan, and topotecan;
(viii) inhibitors of topoisomerase II, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and (xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See. e.g., Wawrzynczak (1996) Antibody Therapy, Bios Scientific Pub. Ltd, Oxfordshire, UK; Kresina (ed.) (1991) Monoclonal Antibodies. Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

The present invention contemplates embodiments of the combination therapy that include a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy may be used to treat one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Additional Combination Therapies

The p53 peptidomimetic macrocycles disclosed herein may be used in combination with other therapies. For example, the combination therapy may include a composition comprising a p53 peptidomimetic macrocycle co-formulated with, and/or co-administered with, one or more additional therapeutic agents, e.g., hormone treatment, vaccines, and/or other immunotherapies. In other embodiments, the p53 peptidomimetic macrocycle is administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The p53 peptidomimetic macrocycle may be administered concurrently with, prior to, or subsequent to, one or more other additional therapies or therapeutic agents. The p53 peptidomimetic macrocycle and the other agent or therapeutic protocol may be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In certain embodiments, a p53 peptidomimetic macrocycle described herein is administered in combination with one or more check point inhibitors or antagonists of programmed death receptor 1 (PD-1) or its ligand PD-L 1 and PD-L2. The inhibitor or antagonist may be an antibody, an antigen binding fragment, an immunoadhesin, a fusion protein, or oligopeptide. In some embodiments, the anti-PD-1 antibody is chosen from nivolumab (OPDIVO, Bristol Myers Squibb, New York, New York), pembrolizumab (KEYTRUDA, Merck Sharp & Dohme Corp, Kenilworth, NJ USA), cetiplimab (Regeneron, Tarrytown, NY) or pidilizumab (CT-011). In some embodiments, the PD-1 inhibitor is an immunoadhesin (e.g., an immunoadhesin comprising an extracellular or PD-1 binding portion of PD-L 1 or PD-L2 fused to a constant region (e.g., an Fc region of an immunoglobulin sequence)). In some embodiments, the PD-1 inhibitor is AMP-224. In some embodiments, the PD-L 1 inhibitor is anti-PD-L 1 antibody such durvalumab (IMFINZI, Astrazeneca, Wilmington, DE), atezolizumab (TECENTRIQ, Roche, Zurich, CH), or avelumab (BAVENCIO, EMD Serono, Billerica, MA). In some embodiments, the anti-PD-L1 binding antagonist is chosen from YW243.55.S70, MPDL3280A, MEDI-4736, MSB-0010718C, or MDX-1105.

The following examples are intended to promote a further understanding of the present invention.

### GENERAL METHODS

### Peptide Synthesis

All peptides were sourced from CPC Scientific. The purity and identity of the peptides was confirmed by analytic HPLC and mass spectrometry. All the final peptides have ≥90% purity. All peptides are dissolved in neat dimethyl sulfoxide (DMSO) as 10 mM stock solution and diluted thereof for subsequent experiments.

Peptides were synthesized using Rink Amide 4-Methylbenzhydrylamine (MBHA) resin and Fmoc-protected amino acids, coupled sequentially with N,N′-diisoproplcarbodiimide/hydroxybenzotrizole (DIC/HOBt) activating agents. Double coupling reactions were performed on the first amino acid and also at the stapling positions. At these latter positions, the activating reagents were switched to N,N-diisopropylethylamine/Hexafluorophosphate Azabenzotriazole Tetramethyl Uronium (DIEA/HATU) for better coupling efficiencies. Ring closing metathesis reactions were performed by first washing the resin three times with DCM (dichloromethane), followed by the addition of the first-generation Grubbs Catalyst (20 mol % in DCM and allowed to react for 2 hours; all steps with Grubbs Catalyst were performed in the dark). The RCM (ring closing metathesis) reaction was repeated to ensure a complete reaction. After the RCM was complete, a test cleavage was performed to ensure adequate yield. Peptides were cleaved and then purified as a mixture of cis-trans isomers by reverse phase-high performance liquid chromatography (RP-HPLC). Glaser alkyne cross-coupling reactions were performed on resin by first washing the resin three times with DCM, followed by the addition of tetrahydrofuran (THF), N,N-Diisopropylethylamine (DIPEA), Pd(PPh₃)₂Cl₂ then CuI, ultrasonication then heating at 30° C. for 16 hours. The mixture was filtered and washed with dimethylformamide (DMF).

### MDM2 Protein Production

For use in the peptide binding assay, a human MDM2 1-125 sequence was cloned into a pNIC-GST vector. The TEV (tobacco etch virus) cleavage site was changed from ENLYFQS (SEQ ID NO: 13) to ENLYFQG (SEQ ID NO: 14) to give a fusion protein with the following sequence:

(SEQ ID NO: 12)
MSDKIIHSPILGYWKIKGLVQPTRLLLEYLEEKYEEHLYERDEGDKWRN
KKFELGLEFPNLPYYIDGDVKLTQSMAIIRYIADKHNMLGGCPKERAEI
SMLEGAVLDIRYGVSRIAYSKDFETLKVDFLSKLPEMLKMFEDRLCHKT
YLNGDHVTHPDFMLYDALDVVLYMDPMCLDAFPKLVCFKKRIEAIPQID
KYLKSSKYIAWPLQGWQATFGGGDHPPKLEVLFQGHMHHHHHHSSGVDL
GTENLYFQGMCNTNMSVPTDGAVTTSQIPASEQETLVRPKPLLLKLLKS
VGAQKDTYTMKEVLFYLGQYIMTKRLYDEKQQHIVYCSNDLLGDLFGVP
SFSVKEHRKIYTMIYRNLVVVNQQESSDSGTSVSEN.

The corresponding plasmid was transformed into BL21 (DE3) Rosetta T1R Escherichia coli cells and grown under kanamycin selection. Bottles of 750 mL Terrific Broth, supplemented with appropriate antibiotics and 100 μL of antifoam 204 (Sigma-Aldrich, St. Louis, MO, USA, were inoculated with 20 mL seed cultures grown overnight. The cultures were incubated at 37° C. in the LEX system (Harbinger Biotech, Toronto, Canada) with aeration and agitation through the bubbling of filtered air through the cultures. The LEX system temperature was reduced to 18° C. when culture OD600 reached 2, and the cultures were induced after 60 minutes with 0.5 mM IPTG. Protein expression was allowed to continue overnight. Cells were harvested by centrifugation at 4000×g, at 15° C. for 10 minutes. The supernatant fractions were discarded and the cell pellets were resuspended in a lysis buffer (1.5 mL per gram of cell pellet). The cell suspensions were stored at −80° C. before purification work.

The re-suspended cell pellet suspensions were thawed and sonicated (Sonics Vibra-Cell, Newtown, CO, USA) at 70% amplitude, 3 seconds on/off for 3 minutes, on ice. The lysate was clarified by centrifugation at 47,000×g, 4° C. for 25 minutes. The supernatant fractions were filtered through 1.2 μm syringe filters and loaded onto the AKTA Xpress system (GE Healthcare, Fairfield, CO, USA). The purification regime is briefly described as follows.

The lysates were loaded onto a 1 mL Ni-NTA Superflow column (Qiagen, Valencia, CA, USA) that had been equilibrated with 10 column volumes of wash 1 buffer. Overall buffer conditions were as follows: Immobilized metal affinity chromatography (IMAC) wash 1 buffer-20 mM HEPES ((4-(2-hydroxyethyl)-1-piperazineethanesulfonic acid), 500 mM NaCl, 10 mM Imidazole, 10% (v/v) glycerol, 0.5 mM TCEP (Tris(2-carboxyethyl)phosphine), pH 7.5; IMAC wash 2 buffer-20 mM HEPES, 500 mM NaCl, 25 mM Imidazole, 10% (v/v) glycerol, 0.5 mM TCEP, pH 7.5; IMAC Elution buffer-20 mM HEPES, 500 mM NaCl, 500 mM Imidazole, 10% (v/v) glycerol, 0.5 mM TCEP, pH 7.5. The sample was loaded until air was detected by the air sensor, 0.8 mL/minutes. The column was then washed with wash 1 buffer for 20 column volumes (CV), followed by 20 CV of wash 2 buffer. The protein was eluted with five CV of elution buffer. The eluted proteins were collected and stored in sample loops on the system and then injected into gel filtration (GF) columns. Elution peaks were collected in 2 mL fractions and analyzed on SDS-PAGE gels. The entire purification was performed at 4° C. Relevant peaks were pooled, TCEP was added to a total concentration of 2 mM. The protein sample was concentrated in Vivaspin 20® filter concentrators (VivaScience, Littleton, MA, USA) at 15° C. to approximately 15 mg/mL (<18 kDa-5 K MWCO, 19-49 kDa-10 K MWCO, >50 kDa-30 K MWCO). The final protein concentration was assessed by measuring absorbance at 280 nm on Nanodrop ND-1000® (Thermo Fisher, Waltham, MA, USA). The final protein purity was assessed on SDS-PAGE gel. The final protein batch was then aliquoted into smaller fractions, frozen in liquid nitrogen and stored at −80° C.

For x-ray crystallography, MDM2 (6-125) was cloned as a GST-fusion protein using the pGEX-6P-1 GST expression vector (GE Healthcare). The GST-fused MDM2 (6-125) construct was then transformed into Escherichia coli BL21(DE3) pLysS (Thermo Fisher, Waltham, MA, USA) competent cells. Cells were grown in Luria-Bertani (LB) medium at 37° C. and induced at OD600 nm of 0.6 with 0.5 mM Isopropyl β-D-1-thiogalactopyranoside (IPTG) at 16° C. After overnight induction, the cells were harvested by centrifugation, resuspended in binding buffer (50 mM Tris-HCl pH 8.0, 150 mM NaCl), and lysed by sonication. After centrifugation for 60 min at 19,000×g at 4° C., the cell lysate was then applied to a 5 mL GSTrap® FF column (GE Healthcare) pre-equilibrated in wash buffer (50 mM Tris-HCl pH 8.0, 150 mM NaCl, 1 mM DTT). The GST-fused MDM2 (amino acids 6-125) was then cleaved on-column by PreScission® protease (GE Healthcare) overnight at 4° C. and eluted off the column with wash buffer. The protein sample was then dialyzed into a buffer A solution (20 mM Bis-Tris, pH 6.5, 1 mM DTT) using HiPrep® 26/10 Desalting column, and loaded onto a cation-exchange Resource S 1 mL column (GE Healthcare), pre-equilibrated in buffer A. The column was then washed in six CV of buffer A and the bound protein was eluted with a linear gradient in buffer comprising 1 M NaCl, 20 mM Bis-Tris pH 6.5, and 1 mM DTT over 30 column volumes. Protein purity as assessed by SDS-PAGE was about 95%, and the proteins were concentrated using Amicon-Ultra (3 kDa MWCO) concentrator (Millipore, Burlington, MA, USA). Protein concentration was determined using 280 nm absorbance measurements.

### MDM4 Protein Production

MDM4 protein was cloned into pNIC-GST vector and expressed in LEX system (Harbinger Biotech) at Protein Production Platform (PPP) at NTU School of Biological Sciences. Using glycerol stocks, inoculation cultures were started in 20 mL Terrific Broth with 8 g/L glycerol supplemented with Kanamycin. The cultures were incubated at 37° C., 200 rpm overnight. The following morning, bottles of 750 mL Terrific Broth with 8 g/L glycerol supplemented with Kanamycin and 100 μL of antifoam 204 (Sigma-Aldrich) were inoculated with the cultures. The cultures were incubated at 37° C. in the LEX system with aeration and agitation through the bubbling of filtered air through the cultures. When the OD600 reached about 2, the temperature was reduced to 18° C. and the cultures were induced after 30 to 60 minutes with 0.5 mM IPTG. Protein expression was allowed to continue overnight. The following morning, cells were harvested by centrifugation at 4200 rpm at 15° C. for 10 minutes. The supernatant fractions were discarded and the cells were re-suspended in lysis buffer (100 mM HEPES, 500 mM NaCl, 10 mM Imidazole, 10% glycerol, 0.5 mM TCEP, pH 8.0 with Benzonase (4 μL per 750 mL cultivation) and 250 U/μL Merck Calbiochem™ Protease Inhibitor Cocktail Set III, EDTA free (1000× dilution in lysis buffer) from Calbiochem) at 200 rpm, 4° C. for approximately 30 minutes and stored at −80° C. The re-suspended cell pellet suspensions were thawed and sonicated (Sonics Vibra-cell) at 70% amplitude, 3 seconds on/off for 3 minutes, on ice. The lysate was clarified by centrifugation at 47000×g, 4° C. for 25 minutes. The supematants were filtered through 1.2 μm syringe filters and loaded onto AKTA Xpress system (GE Healthcare) with a 1 mL Ni-NTA Superflow (Qiagen) IMAC column. The column was washed with 20 column volume (CV) of wash buffer 1 (20 mM HEPES, 500 mM NaCl, 10 mM Imidazole, 10% (v/v) glycerol, 0.5 mM TCEP, pH 7.5) and 20 CV of wash buffer 2 (20 mM HEPES, 500 mM NaCl, 25 mM Imidazole, 10% (v/v) glycerol, 0.5 mM TCEP, pH 7.5) or until a stable baseline for 3 minutes and delta base 5 mAU (0.8 mL/minutes) was obtained respectively. MDM4 protein was eluted with elution buffer (20 mM HEPES, 500 mM NaCl, 500 mM Imidazole, 10% (v/v) glycerol, 0.5 mM TCEP, pH 7.5) and eluted peaks (start collection: >50 mAU, slope >200 mAU/min, stop collection: <50 mAU, stable plateau of 0.5 minutes, delta plateau 5 mAU) were collected and stored in sample loops on the system and then injected into equilibrated Gel Filtration (GF) column (HiLoad 16/60 Superdex 200 prep grade (GE Healthcare)) and eluted with 20 mM HEPES, 300 mM NaCl, 10% (v/v) glycerol, 0.5 mM TCEP, pH 7.5 at a flowrate of 1.2 mL/minutes. Elution peaks (start collection: >20 mAU, slope >10 mAU/minute, stop collection: <20 mAU, slope >10 mAU/minute, minimum peak width 0.5 min) were collected in 2 mL fractions. The entire purification was performed at 4° C. Relevant peaks were pooled and TCEP was added to a final concentration of 2 mM. The protein sample was concentrated in Vivaspin 20 filter concentrators (VivaScience) at 15° C. to approximately 15 mg/mL. The final protein concentration was assessed by measuring absorbance at 280 nm on Nanodrop™ ND-1000 (Nano-Drop Technologies). The final protein purity was assessed by SDS-PAGE and purified MDM4 protein was frozen in liquid nitrogen and stored at −80° C.

### Competitive Fluorescence Anisotropy Assays (MDM2 and MDM4)

Purified MDM2 (1-125) protein was titrated against 50 nM carboxyfluorescein (FAM)-labeled 12/1 peptide 13 (the 9-mer FAM-RFMDYWEGL-NH₂ (SEQ ID NO: 26) disclosed in Fischer, Intl. J. Pept. Res. Ther. 12: 3-19 (2006)). Dissociation constants for titrations of MDM2 and MDM4 against FAM-labeled 12/1 peptide were determined by fitting the experimental data to a 1:1 binding model equation shown below:

$$r = {r}_{0} + ( {r}_{b} - r ) \times \frac{( {K}_{d} + {[ L ]}_{t} + {[ P ]}_{t} ) - \sqrt{{{K}_{d} + {[ L ]}_{t} + {[ P ]}_{t} )}^{2} - {{4 [ L ]}_{t} [ P ]}_{t}}}{{2 [ L ]}_{t}} \qquad Equation 1$$

[P] is the protein concentration (MDM2), [L] is the labeled peptide concentration, r is the anisotropy measured, r₀ is the anisotropy of the free peptide, r_{b} is the anisotropy of the MDM2-FAM-labeled peptide complex, K_{d} is the dissociation constant, [L]ₜ is the total FAM labeled peptide concentration, and [P]ₜ is the total MDM2 concentration. The apparent K_{d} values for FAM-labeled 12/1 peptide against MDM2 and MDM4 were determined to be 13.0 nM and 4.0 nM, respectively. These values were then used to determine apparent K_{d} values of the respective competing ligands in subsequent competition assays in fluorescence anisotropy experiments. MDM2 and MDM4 competition experiments were performed with their respective concentrations held constant at 250 nM and 75 nM, in the presence of 50 nM of FAM-labeled 12/1. The competing molecules were then titrated against the complex of the FAM-labeled peptide and protein. Apparent Kd values were determined by fitting the experimental data to the equations shown below:

$$r = {r}_{0} + ( {r}_{b} - {r}_{0} ) \times \frac{2 \sqrt{( {d}^{2} - 3 e )} cos ( θ / 3 ) - 9}{3 {K}_{d 1} + 2 \sqrt{( {d}^{2} - 3 e )} cos ( θ / 3 ) - d}$$

$$d = {K}_{d 1} + {K}_{d 2} + {[ L ]}_{st} + {[ L ]}_{t} - {[ P ]}_{t}$$

$$e = ( {[ L ]}_{t} - {[ P ]}_{t} ) {K}_{d 1} + ( {[ L ]}_{st} - {[ P ]}_{t} ) {K}_{d 2} + {K}_{d 1} {K}_{d 2}$$

$$f = - {K}_{d 1} {K}_{d 2} [ P ] ,$$

$$θ = ar cos [ \frac{- 2 {d}^{3} + 9 de - 27 f}{2 {( {d}^{2} - 3 e )}^{3}} ]$$

[L]ₛₜ and [L]ₜ denote labeled ligand and total unlabeled ligand input concentrations, respectively. K_{d2} is the dissociation constant of the interaction between the unlabeled ligand and the protein.

In all competition experiments, it is assumed that [P]t>[L]ₛₜ, otherwise considerable amounts of free labeled ligand would always be present and would interfere with measurements. K_{d1} is the apparent K_{d} for the labeled peptide used and has been experimentally determined as described in the previous paragraph. The FAM-labeled peptide was dissolved in dimethyl sulfoxide (DMSO) at 1 mM and diluted into experimental buffer. Readings were carried out with an Envision Multilabel Reader (PerkinElmer). Experiments were carried out in PBS (2.7 mM KCl, 137 mM NaCl, 10 mM Na₂HPO₄ and 2 mM KH₂PO₄ (pH 7.4)) and 0.1% Tween-20 buffer. All titrations were carried out in triplicate. Curve-fitting was carried out using Prism 4.0 (GraphPad®). To validate the fitting of a 1:1 binding model we carefully ensured that the anisotropy value at the beginning of the direct titrations between MDM2 and the FAM-labeled peptide did not differ significantly from the anisotropy value observed for the free fluorescently labeled peptide. Negative control titrations of the ligands under investigation were also carried out with the fluorescently labeled peptide (in the absence of MDM2) to ensure no interactions were occurring between the ligands and the FAM-labeled peptide. In addition, we ensured that the final baseline in the competitive titrations did not fall below the anisotropy value for the free FAM-labeled peptide, which would otherwise indicate an unintended interaction between the ligand and the FAM-labeled peptide to be displaced from the MDM2 binding site. Measurements were taken a minimum of three times (biological replicates) and values are reported as geometric means of the replicates.

### Whole Cell Homogenate Stability

Peptides at a concentration of 1 μM were incubated at 37° C. with HCT116 whole cell homogenates prepared from 1 million lyzed cells/mL. The reaction was stopped at 0, 1, 2, and 4 hours and 22 hours with an organic solvent followed by centrifugation. The resulting supernatant was injected to LC/MS for the detection of tested peptide. The remaining percentage of each compound was normalized to the time 0 hour amount and reported.

### Plasma Stability

Peptide was incubated with human plasma at the concentration of 1 mM at 37° C. for 1, 2, 3, and 4 hours. The incubation was stopped at indicated time points by addition of organic solvent followed by centrifugation. Parent compound in supernatant was analyzed by LC/MS. Percent remaining of peptide was calculated against the amount of compound at time 0.

### p53 Beta-Lactamase Reporter Gene Cellular Functional Assay

HCT116 cells were stably transfected with a p53 responsive β-lactamase reporter and expanded in McCoy's 5A Medium with 10% fetal bovine serum (FBS), Blasticidin, and Penicillin/Streptomycin and then transferred to 1.5 mL freezing vials and stored under liquid nitrogen in growth media containing 5% DMSO. One day prior the assay, a vial of banked cells was recovered in a cell culture flask and incubated for 24 hours, followed by removal of cell growth media and replacement with Opti-MEM containing 2% FBS. The cells were then seeded into a 384-well plate at a density of 8000 cells per well. Peptides were then dispensed to each well using a liquid handler, ECHO 555, and incubated 16 hours. The final working concentration of DMSO was 0.5%. β-lactamase activity was detected using the ToxBLAzer Dual Screen (Invitrogen), as per the manufacturer's instructions. Measurements were made using the Envision multiplate reader (PerkinElmer). Maximum p53 activity was defined as the amount of 0-lactamase activity induced by 50 μM azide-ATSP-7041. This was determined as the highest amount of p53 activity induced by azide-ATSP-7041 from titrations on HCT116 cells. Measurements were taken a minimum of three times (biological replicates) and values are reported as geometric means of the replicates.

### Lactate Dehydrogenase (LDH) Release Assay

HCT116 cells were stably transfected with a p53 responsive β-lactamase reporter and expanded in McCoy's 5A Medium with 10% fetal bovine serum (FBS), Blasticidin, and Penicillin/Streptomycin and then transferred to 1.5 mL freezing vials and stored under liquid nitrogen in growth media containing 5% DMSO. One day prior the assay, a vial of banked cells was recovered in a cell culture flask and incubated for 24 hours, followed by removal of cell growth media and replacement with Opti-MEM containing 2% FBS. The cells were then seeded into a 384-well plate at a density of 8000 cells per well. Peptides were then dispensed to each well using a liquid handler, ECHO 555, and incubated 16 hours. The final working concentration of DMSO was 0.5%. Lactate dehydrogenase release was detected using the CytoTox-ONE Homogenous Membrane Integrity Assay Kit (Promega), as per the manufacturer's instructions. Measurements were carried out using the Tecan plate reader. Maximum LDH release was defined as the amount of LDH released as induced by the lytic peptide (iDNA79) and used to normalize the results. Measurements were taken a minimum of three times (biological replicates) and values are reported as geometric means of the replicates.

### Tetracycline Beta-Lactamase Reporter Gene Cellular Assay (Counterscreen)

This assay was based on Jump-In™ T-REx™ CHO-K1 BLA cells containing a stably integrated β-lactamase under the control of an inducible cytomegalovirus (CMV) promoter. Cells were maintained in Dulbecco's Minimal Eagle Medium (DMEM) with 10% fetal bovine serum (FBS), Blasticidin, and Penicillin/Streptomycin and then transferred to 1.5 mL freezing vials and stored under liquid nitrogen in growth media containing 5% DMSO. One day prior the assay, a vial of banked cells was recovered in a cell culture flask and incubated for 24 hours, followed by removal of cell growth media and replacement with Opti-MEM containing 2% FBS. Cells were seeded into a 384-well plate at a density of 4000 cells per well. Peptides were then dispensed to each well using a liquid handler, ECHO 555 and incubated for 16 h. The final working concentration of DMSO was 0.5%. β-lactamase activity was detected using the ToxBLAzer Dual Screen (Invitrogen), as per the manufacturer's instructions. Measurements were carried out using the Envision multiplate reader (PerkinElmer). Counterscreen activity was defined as the amount of β-lactamase activity induced by tetracycline. Measurements were taken a minimum of three times (biological replicates) and values are reported as geometric means of the replicates.

### HCT-116 Western Blot Analysis

Preparation of compound Stock and working Solutions: 10 mM or 1 mM stock solutions of compounds were prepared in 100% DMSO. Each compound was then serially diluted in 100% DMSO and further diluted 10-fold into HPLC grade sterile water to prepare 10× working solutions in 10% DMSO/water of each compound. Depending on the required volume used in the relevant assay, compounds were added to yield final concentrations as indicated in the relevant figure with a residual DMSO concentration of 1% v/v.

HCT116 cells (Thermo Fisher Scientific) were cultured in DMEM cell media, which was supplemented with 10% foetal calf serum (FBS) and penicillin/streptomycin. All cell lines were maintained in a 37° C. humidified incubator with 5% CO₂ atmosphere. HCT116 cells were seeded into 96 well plates at a cell density of 60,000 cells per well and incubated overnight. Cells were also maintained in DMEM cell media with 10% fetal bovine serum (FBS) and penicillin/streptomycin. Cell media was then removed and replaced with cell media containing the various compounds/vehicle controls at the concentrations indicated in DMEM cell media with 2% FCS. After the stated incubation time (4 or 24 hours) cells were rinsed with PBS and then harvested in 100 μL of 1× NuPAGE LDS sample buffer supplied by Invitrogen (NP0008). Samples were then sonicated, heated to 90° C. for 5 minutes, sonicated twice for 10 seconds and centrifuged at 13, 000 rpm for 5 minutes. Protein concentrations were measured by BCA assay (Pierce). Samples were resolved on Tris-Glycine 4-20% gradient gels (BIORAD) according to the manufacturer's protocol. Western transfer was performed with an Immuno-blot PVDF membrane (Bio-Rad) using a Trans-Blot Turbo system (BIORAD). Western blot staining was then performed using antibodies against actin (AC-15, Sigma) as a loading control, p²¹ (118 mouse monoclonal), MDM2 (2A9 mouse monoclonal antibody) and p53 (DO-1 mouse monoclonal antibody).

### Isothermal Titration Calorimetry (ITC)

Overnight dialysis of protein and peptides were carried out in buffer containing 1× phosphate-buffered saline (PBS) pH 7.2, 3% DMSO, and 0.001% Tween-20. Approximately 100-200 μM of peptide was titrated into 20 μM of purified recombinant human MDM2 protein (amino acids 1-125), over 40 injections of 1 μL each. Reverse ITC (200 μM of MDM2 protein titrated into 20 μM of peptide) was carried out for peptides that are insoluble at high concentrations. All experiments were performed in duplicates using the MicroCal PEAQ-ITC Automated system. Data analysis was carried out using the MicroCal PEAQ-ITC Analysis Software.

### Circular Dichroism (CD)

A total of 5 μL of the 10 mM stock peptide was mixed with 45 μL of 100% methanol and dried for 2 hours in the SpeedVac™ concentrator (Thermo Scientific). The dried peptide was reconstituted in a buffer (1 mM Hepes pH 7.4 and 5% methanol) to a concentration of 1 mM. The peptide sample was placed in a quartz cuvette with a path length of 0.2 cm. The peptide concentration was determined by the absorbance of the peptide at 280 nM. The CD spectrum was recorded from 300 to 190 nm using the Chirascan-plus qCD machine (Applied Photophysics, Surrey, UK), at 25° C. All experiments were done in duplicates. The CD spectrum was converted to mean residue ellipticity before deconvolution and estimation of the secondary structure components of the peptide using the CDNN software (distributed by Applied Photophysics). Measurements were taken at least twice and were reported as arithmetic means.

### EXAMPLE 1

Improvement of a ^{D}PMI-δ (6-10) olefin stapled p53 peptidomimetic macrocycle that had poor Cellular Activity and Both LDH and Counterscreen Activity

WO2020257153A1 describes a series of ^{D}PMI-δ olefin stapled peptides that showed improved cellular activity as compared to the parent peptide (see also Chem Sci. 2020, 11, 5577). We focused on the DPMI-δ (6-10) olefin stapled p53 peptidomimetic macrocycle since it had innate hyperstability, verified binding to MDM2, and an amphipathic character. However, it showed poor cellular activity and cellular activity liabilities in both LDH and counterscreen assays (compound 1, Table 1, parts 1-3). Those liabilities were likely due to the peptides three positive charges, including a free N-terminus, and two basic residues (D-Lysine at position 9 and D-Arginine at position 12). Using ATSP-7041 as an amphipathic reference with good cell permeability and activity, we noted the presence of Ser and Gin residues on its polar helical face. To remove the positive charges on DPMI-δ (6-10) olefin stapled p53 peptidomimetic macrocycle, we thus introduced D-Glutamine at position 9 and D-Serine at position 12 along with N-terminal acetylation. Those modifications were successful in removing LDH-release and counterscreen activities (both EC50s >50 μM) and improved p53 reporter gene functional cellular activity by 3-fold to 3.6 μM at 0% serum (compound 2). However, the compound showed weak cellular efficacy under 10% serum conditions (>50 μM).

### EXAMPLE 2

### Appending a C-Terminal Tail to DPMI™ (6-10) Olefin Stapled pS3 Peptidomimetic Macrocycle Resulted in Improved Cellular Update and Physiochemical Properties

The introduction of a polypeptide tail comprising several D-alanine residues could further enhance cellular activity for our DPMI-δ(6-10) olefin stapled p53 peptidomimetic macrocycles possibly by modulating the helical propensity of the added poly-D-alanine tail. This could both increase solubility and alter the dynamics of solution-state peptide oligomers through favoring the α-helical conformation by mitigating β-sheet based peptide aggregation. In addition, enhanced aqueous-phase helicity could enhance permeability by decreasing the entropic penalty related to the adaptation of the high helical content that is typically induced in the low dielectric constant of the cell membrane lipid bilayer. We made a series of compound 2 analogs appended with polypeptide tails comprising different numbers of D-alanine residues (compounds 3 to 5, Table 1, parts 1-3). Of the three tail lengths evaluated, the 6 amino acid length provided the best profile (compound 4): appending a 6×(D-Ala) C-terminal tail improved cellular activity to 0.3 μM in 10% serum for an overall improvement of 100-fold, compared to the initial parent peptide. Interestingly, for compound 2, further extension of the length of the polyalanine tail to 9×(D-Ala) led to a compound 2 analog with poorer cellular efficacy (compound 5).

Comparing the physicochemical properties of these analogs with different polyalanine size length provides a possible explanation as compound 5 is much less soluble than analogs appended with 3×(D-Ala) or 6×(D-Ala) polypeptide tails (16 μM vs 140 μM or 150 μM, respectively) and less soluble than the parent compound 2 (16 μM vs 167 μM) (Table 2). Similarly, compound 5 also showed higher measured HPLC logD as compared to compounds 2 to 4, which could result in poorer solution behavior including aggregation.

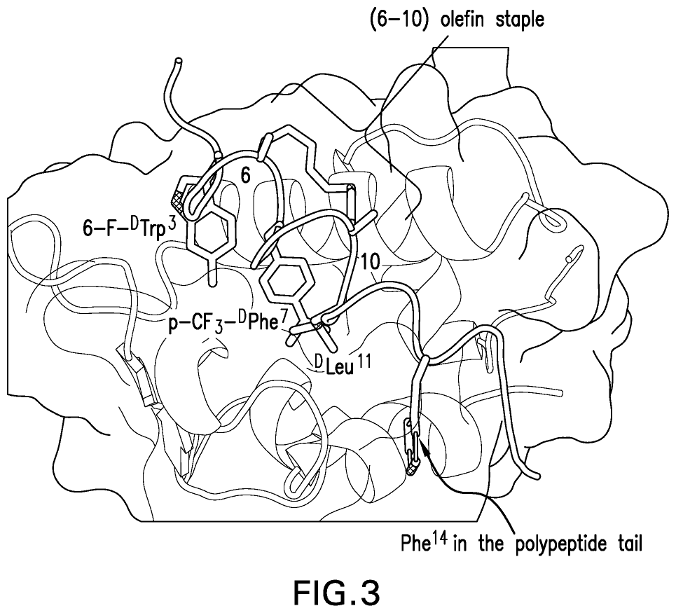

We sought to further enhance cell potency while avoiding off-target effects by optimizing the polypeptide tail sequence. Our multi-pronged approach involved optimizing MDM2(X) binding through additional binding interactions in the tail as well as optimization of solubility and solution state behavior through enhanced amphipathicity to balance the hydrophobic character. Examination of the helical wheel revealed that placement of apolar residues at position 14 might enhance MDM2 binding, further molecular modeling suggested that phenylalanine might be an appropriate residue for that position (FIG. 3). Accordingly, we made compound 6 with a polyalanine tail wherein the D-Ala at position 14 has been replaced with D-Phe This peptide exhibited about 65% helicity in aqueous solution.

Compound 6 retained some cellular activity as compared to parent compound 2, which lacks a polypeptide tail, but proved to be equivalent to compound 4, a result that could be related to the high measured HPLC logD for the compound (Table 1, parts 1-3, and Table 2). To enhance peptide solubility, we also probed for amino acid positions that would tolerate polar residues without compromising cellular activity. Examination of the helical wheel (see Chem Sci. 2020, 11, 5577 for description of helical structures) revealed that placement of apolar residues at amino acid position 16 may be tolerated. Accordingly, we made compound 7 with a polypeptide tail of six D-Ala but wherein D-Ala at amino acid position 16 had been replaced with D-Glu. This peptide improved cellular activity versus compound 6 in 0% and 10% serum (1.41 μM and 0.79 μM vs 9.65 μM and 1.75 μM). Although its cellular activity is somewhat right shifted versus compound 4, the glutamic acid can provide advantage for less soluble scaffolds.

### EXAMPLE 3

Using a Specific Cross-Link (i, i+7 Bis-Alkyne at Position 5 and 12) to Replace the Olefin in ^{D}PMI-δ-(5-12) Olefin Staplde p53 Peptidomimetic Macrocycle Provided Improved Rigidity and Alpha-Helicity

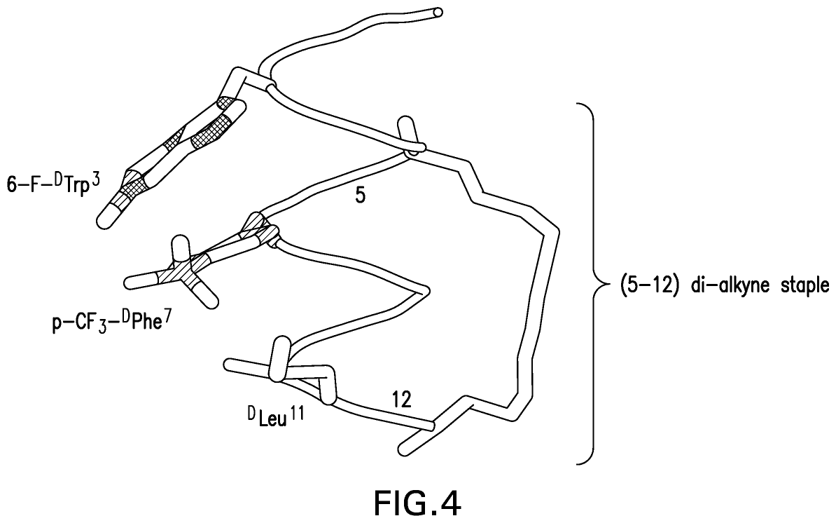

We also explored introducing a different staple type in our ^{D}PMI-δ series to stabilize the helical conformation as a means to improve counterscreen (off target) activity and solubility. Previously reported ^{D}PMI-δ-(5-12) olefin stapled p53 peptidomimetic macrocycle (see WO2020257153 and Chem Sci. 2020, 11, 5577) was a good candidate since the compound showed modest cellular activity and some cellular activity liability in the counterscreen assay. Attempts to determine solubility and HPLC logD had also failed, likely due to its poor solubility (compound 8, Table 1, parts 1-3, and Table 2). A di-alkyne staple replacement for the i, i+7 olefin staple was made since such a replacement could provide an optimal distance between the β-carbons of the i, i+7 amino acid side chains, resulting in a stabilized α-helical structure (see Chembiochem 2018; 19, 1031). The di-alkyne staple resulted in a single conformation for the helix, greatly stabilizing its structure (FIG. 4). Predicted helicity also showed an improvement (FIG. 4). This peptide exhibited increased helicity about 60% (corresponding hydrocarbon staple exhibited about 32% helicity)

### EXAMPLE 4

Combination of the ^{D}PMI-δ-(5-12) Di-Alkyne Stapled p53 Peptidomimetic Macrocycle with Certain Other Amino Acid Substitutions and a Polypeptide Tail Improved Cellular Update and Physiochemical Properties

We modified the ^{D}PMI-δ-(5-12) di-alkyne stapled p53 peptidomimetic macrocycle by (i) introducing D-Gln at amino acid position 9 along with N-terminal acetylation to remove the positive charges by analogy with our changes in ^{D}PMI-δ-(6-10) olefin stapled p53 peptidomimetic macrocycle and (ii) linking a 6× (D-Ala) polypeptide tail at the C-terminus of the to optimize solubility and helicity. Overall, those changes produced compound 9 that showed greatly improved cellular activity to 0.3 μM in 10% serum for an overall improvement of 100-fold as compared to the parent peptide compound 8 (Table 1, parts 1-3). The compound was also cleaner than the parent in counterscreen assay (Table 1, parts 1-3) and solubility could be measured (Table 2). Compound 9 demonstrates the applicability of modifications reported in this invention.

### EXAMPLE 5

Compounds Very Clean in Counterscreens could be Obtained by Applying the Above Modifications

To further establish the impact of the above modification in making cleaner “drug” like compounds, we profiled a set of compounds of the invention in cellular proliferation assays (Table 3). Reference compound 2 showed modest 9.1 μM efficacy in HTC116 positive control cell line, as well as some level of activity in p53 null negative control cell line manifested by 88% inhibition at maximum concentration. By contrast, compound 4, improved cell efficacy by a factor of greater than 6-fold and reduced activity in p53 null negative control cell line to 66% inhibition at maximum concentration. Compound 9 showed sub-micromolar cellular activity in HTC116 positive control lines (on-target assay) and lack of liability in the p53 null negative control cell line (off-target assay) (0.65 μM, and −0.5% inhibition at maximum concentration, respectively).

The compounds reported herein demonstrate the applicability of various modifications disclosed herein to ^{D}PMI-δ p53 peptidomimetic peptides to provide potent, cellularly active, and clean p53 peptidomimetic macrocycles.

TABLE 1
Part 1
Binding and Cellular Activity

| | | | p53 activity | p53 activity |
|---|---|---|---|---|
| | | FP Kd | EC₅₀ (μM) | EC₅₀ (μM) |
| # | Compound Name | MDM2 (nM) | (0% FBS, 4 h) | (10% FBS, 16 h) |
| 1 | ^{D}PMI-δ-(6-10 olefin staple)-NH₂ | 118 | 10.1 | >50 |
| 2 | Acetyl-^{D}PMI-δ-(6-10 olefin staple) | 258 | 3.6 | >50 |
| | (D-K9D-Q, D-R12D-S)-NH₂ | | | |
| 3 | Acetyl-^{D}PMI-δ-(6-10 olefin staple) | 3.1 | 1.44 | 4.34 |
| | (D-K9D-Q, D-R12D-S, 3x(D-A) polypeptide tail)-NH₂ | | | |
| 4 | Acetyl-^{D}PMI-δ-(6-10 olefin staple) | 3.2 | 0.61 | 0.28 |
| | (D-K9D-Q, D-R12D-S, 6x(D-A) polypeptide tail)-NH₂ | | | |
| 5 | Acetyl- ^{D}PMI-δ-(6-10 olefin staple) | 21.7 | 50 | >50 |
| | (D-K9D-Q, D-R12D-S, 9x(D-A) polypeptide tail)-NH₂ | | | |
| 6 | Acetyl-^{D}PMI-δ-(6-10 olefin staple) | 30 | 9.65 | 1.75 |
| | (D-K9D-Q, D-R12D-S) (D-A)-(D-F)-(D-A)-(D-Ala)-(D-A)-(D-A) | | | |
| | polypeptide tail)-NH₂ | | | |
| 7 | Acetyl-^{D}PMI-δ-(6-10 olefin staple) | 0.43 | 1.41 | 0.79 |
| | (D-K9D-Q, D-R12D-S, 3x(D-Ala)-(D-Glu)-2x(D-Ala) | | | |
| | polypeptide tail)-NH₂ | | | |
| 8 | ^{D}PMI-δ-(5-12 olefin staple)-NH₂ | 33.9 | 13.6 | 30.8 |
| 9 | Acetyl-^{D}PMI-δ-(5-12 alkyne staple) | 0.63 | 3.97 | 0.30 |
| | (D-K9D-Q, 6x(D-Ala) polypeptide tail)-NH₂ | | | |
| 10 | Acetyl-^{D}PMI-δ-(5-12 alkyne staple) | 0.12 | 9.47 | 5.0 |
| | (D-K9D-Q, (D-E)-5x(D-A) polypeptide tail)-NH₂ | | | |

TABLE 1
Part 2
Binding and Cellular Activity

| | | LDH release | LDH release |
|---|---|---|---|
| | | EC₅₀ (μM) | EC₅₀ (μM) |
| # | Compound Name | (0% FBS, 4 h) | (10% FBS, 16 h) |
| 1 | ^{D}PMI-δ-(6-10 olefin staple)-NH₂ | 15.3 | 12.6 |
| 2 | Acetyl-^{D}PMI-δ-(6-10 olefin staple) | >50 | >50 |
| | (D-K9D-Q, D-R12D-S)-NH₂ | | |
| 3 | Acetyl-^{D}PMI-δ-(6-10 olefin staple) | >50 | >50 |
| | (D-K9D-Q, D-R12D-S, 3x(D-A) polypeptide tail)-NH₂ | | |
| 4 | Acety1-^{D}PMI-δ-(6-10 olefin staple) | >50 | >50 |
| | (D-K9D-Q, D-R12D-S, 6x(D-A) polypeptide tail)-NH₂ | | |
| 5 | Acetyl-^{D}PMI-δ-(6-10 olefin staple) | >50 | >50 |
| | (D-K9D-Q, D-R12D-S, 9x(D-A) polypeptide tail)-NH₂ | | |
| 6 | Acetyl-^{D}PMI-δ-(6-10 olefin staple) | >50 | >50 |
| | (D-K9D-Q, D-R12D-S) (D-A)-(D-F)-(D-A)-(D-Ala)-(D-A)-(D-A) | | |
| | polypeptide tail)-NH₂ | | |
| 7 | Acetyl-^{D}PMI-δ-(6-10 olefin staple) | >50 | >50 |
| | (D-K9D-Q, D-R12D-S, 3x(D-Ala)-(D-Glu)-2x(D-Ala) | | |
| | polypeptide tail)-NH₂ | | |
| 8 | ^{D}PMI-δ-(5-12 olefin staple)-NH₂ | >50 | >50 |
| 9 | Acetyl-^{D}PMI-δ-(5-12 alkyne staple) | >50 | >50 |
| | (D-K9D-Q, 6x(D-Ala) polypeptide tail)-NH₂ | | |
| 10 | Acetyl-^{D}PMI-δ-(5-12 alkyne staple) | >50 | >50 |
| | (D-K9D-Q, (D-E)-5x(D-A) polypeptide tail)-NH₂ | | |

TABLE 1
Part 3
Binding and Cellular Activity

| | | Counter screen | Counter screen |
|---|---|---|---|
| | | EC₅₀ (μM) | EC₅₀ (μM) |
| # | Compound Name | (0% FBS, 4 h) | (10% FBS, 16 h) |
| 1 | ^{D}PMI-δ-(6-10 olefin staple)-NH₂ | 10.7 | >50 |
| 2 | Acetyl-^{D}PMI-δ-(6-10 olefin staple) | >50 | >50 |
| | (D-K9D-Q, D-R12D-S)-NH₂ | | |
| 3 | Acetyl-^{D}PMI-δ-(6-10 olefin staple) | >50 | >50 |
| | (D-K9D-Q, D-R12D-S, 3x(D-A) polypeptide tail)-NH₂ | | |
| 4 | Acetyl-^{D}PMI-δ-(6-10 olefin staple) | >50 | >50 |
| | (D-K9D-Q, D-R12D-S, 6x(D-A) polypeptide tail)-NH₂ | | |
| 5 | Acetyl-^{D}PMI-δ-(6-10 olefin staple) | >50 | >50 |
| | (D-K9D-Q, D-R12D-S, 9x(D-A) polypeptide tail)-NH₂ | | |
| 6 | Acetyl-^{D}PMI-δ-(6-10 olefin staple) | >50 | >50 |
| | (D-K9D-Q, D-R12D-S) (D-A)-(D-F)-(D-A)-(D-Ala)-(D-A)-(D-A) | | |
| | polypeptide tail)-NH₂ | | |
| 7 | Acetyl-^{D}PMI-δ-(6-10 olefin staple) | >50 | >50 |
| | (D-K9D-Q, D-R12D-S, 3x(D-Ala)-(D-Glu)-2x(D-Ala) | | |
| | polypeptide tail)-NH₂ | | |
| 8 | ^{D}PMI-δ-(5-12 olefin staple)-NH₂ | >50 | >50 |
| 9 | Acetyl-^{D}PMI-δ-(5-12 alkyne staple) | >50 | >50 |
| | (D-K9D-Q, 6x(D-Ala) polypeptide tail)-NH₂ | | |
| 10 | Acetyl-^{D}PMI-δ-(5-12 alkyne staple) | >50 | >50 |
| | (D-K9D-Q, (D-E)-5x(D-A) polypeptide tail)-NH₂ | | |

TABLE 2
Physicochemical Properties

| | | | | | Solubility |
|---|---|---|---|---|---|
| | | CD | | HPLC | (μM) |
| # | Compound Name | (% helicity) | AlogP98 | LogD | (PBS, pH 7) |
| 1 | ^{D}PMI-δ-(6-10 olefin staple)-NH₂ | 38.6 | −1.83 | 2.26 | 74 |
| 2 | Acetyl-^{D}PMI-δ-(6-10 olefin staple) | | −0.62 | 2.35 | 167 |
| | (D-K9D-Q, R12D-S)-NH₂ | | | | |
| 3 | Acetyl-^{D}PMI-δ-(6-10 olefin staple) | | −1.99 | 2.47 | 140 |
| | (D-K9D-Q, D-R12D-S, 3x (D-A) polypeptide tail)-NH₂ | | | | |
| 4 | Acetyl-^{D}PMI-δ-(6-10 olefin staple) | | −3.36 | 2.67 | 150 |
| | (D-K9D-Q, D-R12D-S, 6x (D-A) polypeptide tail)-NH₂ | | | | |
| 5 | Acetyl-^{D}PMI-δ-(6-10 olefin staple) (D- | | −4.73 | 2.89 | 16 |
| | K9D-Q, D-R12D-S, 9x (D-A) polypeptide tail)-NH₂ | | | | |
| 6 | Acetyl-^{D}PMI-δ-(6-10 olefin staple) | | −1.80 | 3.06 | 146 |
| | (D-K9D-Q, D-R12D-S) (D-A)-(D-F)-(D-A)-(D-A)-(D-A)-(D-A) | | | | |
| | polypeptide tail)- NH₂ | | | | |
| 7 | Acetyl-^{D}PMI-δ-(6-10 olefin staple) | | −6.06 | data | data |
| | (D-K9D-Q, D-R12D-S, 3x (D-A)-(D-E)-2x(D-A) | | | pending | pending |
| | polypeptide tail)-NH₂ | | | | |
| 8 | ^{D}PMI-δ-(5-12 olefin staple)-NH₂ | 31.8 | 0.43 | | |
| 9 | Acetyl-^{D}PMI-δ-(5-12 alkyne staple) | 38.9 | −1.32 | 2.12 | 9 |
| | (D-K9D-Q, 6x (D-A) polypeptide tail)-NH₂ | | | | |
| 10 | Acetyl-^{D}PMI-δ-(5-12 alkyne staple) | 38.9 | −3.09 | data | data |
| | (D-K9D-Q, (D-E)-5x (D-A) polypeptide tail)-NH₂ | | | pending | pending |

(*) could not be measured, often the consequence of weak solubility.

TABLE 3
Cellular Proliferation Activity

| | | | | p53 null | p53 null |
|---|---|---|---|---|---|
| | | HCT116 | HCT116 | Ca Ski | Ca Ski |
| | | proliferation | proliferation | proliferation | proliferation |
| | | EC₅₀ | % Max | EC50 | % Max |
| | | (μM, 72 hours, | (μM, 72 hours, | (nM, 72 hours, | (nM, 72 hours, |
| # | Compound Name | 10% serum) | 10% serum) | 10% serum) | 10% serum) |
| 2 | Acetyl-^{D}PMI-δ-(6-10 olefin staple) | 9.1 | 80% | 30.4 | 85% |
| | (D-K9D-Q, D-R12D-S)-NH₂ | | | | |
| 4 | Acetyl-^{D}PMI-δ-(6-10 olefin staple) | 1.48 | 84.5% | 31.1 | 66% |
| | (D-K9D-Q, D-R12D-S, 6x (D-A) | | | | |
| | polypeptide tail)-NH₂ | | | | |
| 9 | Acetyl-^{D}PMI-δ-(5-12 alkyne staple) | 0.65 | 73% | >49.8 | −0.5% |
| | (D-K9D-Q, 6x (D-A) polypeptide tail)-NH₂ | | | | |

### SUMMARY

Contemporary display technologies such as phage and mRNA display readily provide binders to proteins of interest (POI) in vitro. Translation of these in vitro binders into intracellularly active and in vivo active compounds with on target selectivity and specificity is difficult.

The present invention demonstrates the conversion of a toxic (poor cellular activity with counterscreen activity and LDH release) p53 peptidomimetic macrocycles such as ^{D}PMI-δ-(6-10) olefin stapled p53 peptidomimetic macrocycles into an all D-peptide with improved pharmacological properties by adding a polypeptide tail at the C-terminus of the miacrocycle. Further improvements were achieved by replacing the D-Lys at amino acid position 9 with D-Gln and replacing the D-Arg at amino acid position 12 with D-Ser.

The present invention also demonstrates improvement of ^{D}PMI-δ-(5-12) olefin stapled p53 peptidomimetic macrocycles through replacement of the olefin staple with a di-alkyne staple and a polypeptide tail at the C-terminus of the macrocycle, which resulted in an improved compound with much cleaner off target profile. Further improvements were achieved by replacing the D-Lys at amino acid position 9 with D-Gln.

TABLE 4
Sequences

| SEQ | | |
|---|---|---|
| ID | | |
| NO: | Description | Sequence |
| 1 | DPMI delta (6-10 olefin staple)-NH₂ | TAXYAXXEKXLR |
| | wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-12 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 2 | Acetyl-DPMI delta (6-10 olefin staple) | TAXYAXXEQXLS |
| | (K9Q, R12S)-NH₂ wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-12 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 3 | Acetyl-DPMI delta (6-10 olefin staple) | TAXYAXXEQXLSAAA |
| | (K9Q, R12S, 3x(D-Ala)tail)-NH₂ | |
| | wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-15 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 4 | Acetyl-DPMI delta (6-10 olefin staple) | TAXYAXXEQXLSAAAAAA |
| | (K9Q, R12S, 6x(D-Ala)tail)-NH₂ | |
| | wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-18 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 5 | Acetyl-DPMI delta (6-10 olefin staple) | TAXYAXXEQXLSAAAAAAAAA |
| | (K9Q, R12S, 9x(D-Ala)tail)-NH₂ | |
| | wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-21 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 6 | Acetyl-DPMI delta (6-10 olefin staple) | TAXYAXXEQXLSAFAAAA |
| | (K9Q, R12S)(D-Ala)-(D-Phe)-(D- | |
| | Ala)-(D-Ala)-(D-Ala)-(D-Ala)tail)- | |
| | NH₂ wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-18 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 7 | Acetyl-DPMI delta (6-10 olefin staple) | TAXYAXXEQXLSAAAEAA |
| | (K9Q, R12S, 3x(D-Ala)-(D-Glu)-2x(D- | |
| | Ala)tail)-NH₂ wherein | |
| | X³ is 6-F-Trp; | |
| | X⁶ is (R)-2-amino-2-methylhept-6- | |
| | enoic acid; | |
| | X⁷ is p-CF₃-Phe; | |
| | X¹⁰ is (R)-2-amino-2-methylhept-6- | |
| | enoic acid; | |
| | Amino acids 1-18 are D-amino acids; | |
| | X⁶ and X¹⁰ are linked together in an | |
| | olefin linkage; | |
| | the N-terminus is conjugated to an | |
| | acetyl group; | |
| | the C-terminal amino acid comprises an | |
| | amino group | |
| 8 | DPMI delta (5-12 olefin staple)-NH₂ | TAXYXNXEKLLX |
| | wherein | |
| | X³ is D-6-Fluoro-Trp; | |
| | X⁵ is (S)-2-amino-2-methyldec-9-enoic | |
| | acid; | |
| | X⁷ is D-p-CF₃-Phe; | |
| | X¹² is (R)-2-amino-2-methylhept-6- | |
| | enoic acid; | |
| | Amino acids 1-12 are D-amino acids; | |
| | X⁵ and X¹² are linked together in di- | |
| | alkyne linkage; | |
| | the N-terminus is conjugated to an | |
| | acetyl group; | |
| | the C-terminal amino acid comprises an | |
| | amino group | |
| 9 | Acetyl-DPMI delta (5-12 alkyne staple) | TAXYXNXEQLLXAAAAAA |
| | (K9Q, 6x(D-Ala)tail)-NH₂ wherein | |
| | X³ is D-6-Fluoro-Trp; | |
| | X⁵ is (S)-2-amino-2-methylhept-6- | |
| | ynoic acid; | |
| | X⁷ is D-p-CF₃-Phe; | |
| | X¹² is (S)-2-amino-2-methyloct-6- | |
| | ynoic acid; | |
| | Amino acids 1-18 are D-amino acids; | |
| | X⁵ and X¹² are linked together in di- | |
| | alkyne linkage; | |
| | the N-terminus is conjugated to an | |
| | acetyl group; | |
| | the C-terminal amino acid comprises an | |
| | amino group; | |
| 10 | Acetyl-DPMI delta (5-12 alkyne staple) | TAXYXNXEQLLXEAAAAA |
| | (K9Q, (D-Glu)-5x(D-Ala)tail)-NH₂ | |
| | wherein | |
| | X³ is D-6-Fluoro-Trp; | |
| | X⁵ is (S)-2-amino-2-methylhept-6- | |
| | ynoic acid; | |
| | X⁷ is D-p-CF₃-Phe; | |
| | X¹² is (S)-2-amino-2-methyloct-6- | |
| | ynoic acid; | |
| | Amino acids 1-18 are D-amino acids; | |
| | X⁵ and X¹² are linked together in di- | |
| | alkyne linkage; | |
| | the N-terminus is conjugated to an | |
| | acetyl group; | |
| | the C-terminal amino acid comprises an | |
| | amino group; | |
| 11 | ^{D}PMI-delta wherein | TAXYANXEKLLR |
| | X³ is D-6-Fluoro-Trp; | |
| | X⁷ is D-p-CF₃-Phe | |
| 12 | human MDM2 1-125 sequence | MSDKIIHSPILGYWKIKGLVQPTRLLLE |
| | comprising a modified TEV cleavage | YLEEKYEEHLYERDEGDKWRNKKFEL |
| | site | GLEFPNLPYYIDGDVKLTQSMAIIRYIA |
| | | DKHNMLGGCPKERAEISMLEGAVLDI |
| | | RYGVSRIAYSKDFETLKVDFLSKLPEM |
| | | LKMFEDRLCHKTYLNGDHVTHPDFML |
| | | YDALDVVLYMDPMCLDAFPKLVCFK |
| | | KRIEAIPQIDKYLKSSKYIAWPLQGWQ |
| | | ATFGGGDHPPKLEVLFQGHMHHHHH |
| | | HSSGVDLGTENLYFQGMCNTNMSVPT |
| | | DGAVTTSQIPASEQETLVRPKPLLLKLL |
| | | KSVGAQKDTYTMKEVLFYLGQYIMTK |
| | | RLYDEKQQHIVYCSNDLLGDLFGVPSF |
| | | SVKEHRKIYTMIYRNLVVVNQQESSDS |
| | | GTSVSEN |
| 13 | TEV cleavage site | ENLYFQS |
| 14 | Modified TEV cleavage site | ENLYFQG |
| 15 | Human native p53 | MEEPQSDPSVEPPLSQETFSDLWKLLP |
| | | ENNVLSPLPSQAMDDLMLSPDDIEQW |
| | | FTEDPGPDEAPRMPEAAPRVAPAPAAP |
| | | TPAAPAPAPSWPLSSSVPSQKTYQGSY |
| | | GFRLGFLHSGTAKSVTCTYSPALNKMF |
| | | CQLAKTCPVQLWVDSTPPPGTRVRAM |
| | | AIYKQSQHMTEVVRRCPHHERCSDSD |
| | | GLAPPQHLIRVEGNLRVEYLDDRNTFR |
| | | HSVVVPYEPPEVGSDCTTIHYNYMCNS |
| | | SCMGGMNRRPILTIITLEDSSGNLLGRN |
| | | SFEVHVCACPGRDRRTEEENLRKKGEP |
| | | HHELPPGSTKRALSNNTSSSPQPKKKP |
| | | LDGEYFTLQIRGRERFEMFRELNEALE |
| | | LKDAQAGKEPGGSRAHSSHLKSKKGQ |
| | | STSRHKKLMFKTEGPDSD |
| 16 | p53 peptide (amino acids 19-29) of | SQETFSDLWKLLPEN |
| | native human p53 | |
| 17 | A 6-10 olefin stapled p53 | TAXYAXXEKXXXXXXXXX |
| | peptidomimetic macrocycle wherein | |
| | X³ is D-Trp, D-6-Fluoro-Trp, D-6- | |
| | Chloro-Trp, D-6-Bromo-Trp, D-6- | |
| | Iodo-Trp, D-6-methyl-Trp, D-6-cyano- | |
| | Trp, D-6-hydroxy-Trp, D-6-NO₂-Trp, | |
| | D-7-Fluoro-Trp, D-7-Chloro-Trp, D-7- | |
| | Bromo-Trp, D-7-Iodo-Trp, D-7- | |
| | methyl-Trp, D-7-cyano-Trp, D-7- | |
| | hydroxy-Trp, D-7-NO₂-Trp, D-6,7- | |
| | Fluoro-Trp, D-6,7-Chloro-Trp, D-6,7- | |
| | Bromo-Trp, D-6,7-Iodo-Trp, D-6,7- | |
| | methyl-Trp, D-6,7-cyano-Trp, D-6,7- | |
| | hydroxy-Trp, or D-6,7-NO₂-Trp; | |
| | X⁶ is (R)-2-amino-2-methylhept-6- | |
| | enoic acid; | |
| | X⁷ is D-p-Fluoro-Phe, D-p-Chloro-Phe, | |
| | D-p-Bromo-Phe, D-p-Iodo-Phe, D-p- | |
| | methyl-Phe, D-p-cyano-Phe, D-p- | |
| | hydroxy-Phe, D-p-NO₂-Phe, or D-p- | |
| | CF₃-Phe; | |
| | X¹⁰ is (R)-2-amino-2-methylhept-6- | |
| | enoic acid; | |
| | X¹¹ is D-Leu; | |
| | X¹² is D-Arg or D-Ser; | |
| | X¹³ is D-Ala, D-Glu, or D-Gla | |
| | (gamma-carboxylic glutamic acid); | |
| | X¹⁴ is D-Ala; | |
| | X¹⁵ is D-Ala, D-Phe, or D-Glu; | |
| | X¹⁶ is D-Ala or absent; | |
| | X¹⁷ is D-Ala, D-a-methyl-Glu, or | |
| | absent; | |
| | X¹⁸ is D-Ala or absent; | |
| | X¹⁹ is D-Ala or absent; | |
| | X²⁰ is D-Ala or absent; | |
| | X²¹ is D-Ala or absent; | |
| | The N-terminus is optionally | |
| | conjugated to an acyl group having the | |
| | formula RCO− wherein R is an alkane | |
| | having the formula CₙH₂ₙ₊₁ wherein | |
| | n is an integer from 1 to 10; | |
| | The C-terminal amino acid optionally | |
| | comprises an amino group; | |
| | amino acids 1-21 are D amino acids; | |
| | the staple is an olefin obtained through | |
| | ring-closing metathesis between X⁶ and | |
| | X¹⁰ | |
| 18 | A 5-12 di-alkyne stapled p53 | TAXYXNXEXI |
| | peptidomimetic macrocycle wherein | |
| | X³ is D-Trp, D-6-Fluoro-Trp, D-6- | |
| | Chloro-Trp, D-6-Bromo-Trp, D-6- | |
| | Iodo-Trp, D-6-methyl-Trp, D-6-cyano- | |
| | Trp, D-6-hydroxy-Trp, D-6-NO₂-Trp, | |
| | D-7-Fluoro-Trp, D-7-Chloro-Trp, D-7- | |
| | Bromo-Trp, D-7-Iodo-Trp, D-7- | |
| | methyl-Trp, D-7-cyano-Trp, D-7- | |
| | hydroxy-Trp, D-7-NO₂-Trp, D-6,7- | |
| | Fluoro-Trp, D-6,7-Chloro-Trp, D-6,7- | |
| | Bromo-Trp, D-6,7-Iodo-Trp, D-6,7- | |
| | methyl-Trp, D-6,7-cyano-Trp, D-6,7- | |
| | hydroxy-Trp, or D-6,7-NO₂-Trp; | |
| | X⁵ is (S)-2-amino-2-methyldec-9-enoic | |
| | acid; | |
| | X⁷ is D-p-Fluoro-Phe, D-p-Chloro-Phe, | |
| | D-p-Bromo-Phe, D-p-Iodo-Phe, D-p- | |
| | methyl-Phe, D-p-cyano-Phe, D-p- | |
| | hydroxy-Phe, D-p-NO₂-Phe, or D-p- | |
| | CF₃-Phe; | |
| | X9 is D-Lys or D-Gln; | |
| | X¹¹ is D-Leu; | |
| | X¹² is (R)-2-amino-2-methylhept-6- | |
| | enoic acid; | |
| | X¹³ is D-Ala, D-Glu, D-Gla (gamma- | |
| | carboxylic glutamic acid); | |
| | X¹⁴ is D-Ala; | |
| | X¹⁵ is D-Ala, D-Phe, D-Glu; | |
| | X¹⁶ is D-Ala; | |
| | X¹⁷ is D-Ala, D-alpha-methyl-Glu; | |
| | X¹⁸ is D-Ala; | |
| | X¹⁹ is D-Ala or absent; | |
| | X²⁰ is D-Ala or absent; | |
| | X²¹ is D-Ala or absent; | |
| | the N-terminus is optionally conjugated | |
| | to an acyl group having the formula | |
| | RCO−; R is an alkane having the | |
| | formula CₙH₂ₙ₊₁; n is an integer | |
| | from 1 to 10; | |
| | the C-terminal amino acid optionally | |
| | comprises an amino group; amino acids | |
| | 1-21 are D amino acids; | |
| | and the staple is a di-alkyne obtained | |
| | through alkyne cross-coupling between | |
| | X⁵ and X¹² | |
| 19 | A 5-12 di-alkyne stapled p53 | TAXYXNXEXLXX |
| | peptidomimetic macrocycle wherein | |
| | X³ is D-Trp, D-6-Fluoro-Trp, D-6- | |
| | Chloro-Trp, D-6-Bromo-Trp, D-6- | |
| | Iodo-Trp, D-6-methyl-Trp, D-6-cyano- | |
| | Trp, D-6-hydroxy-Trp, D-6-NO₂-Trp, | |
| | D-7-Fluoro-Trp, D-7-Chloro-Trp, D-7- | |
| | Bromo-Trp, D-7-Iodo-Trp, D-7- | |
| | methyl-Trp, D-7-cyano-Trp, D-7- | |
| | hydroxy-Trp, D-7-NO₂-Trp, D-6,7- | |
| | Fluoro-Trp, D-6,7-Chloro-Trp, D-6,7- | |
| | Bromo-Trp, D-6,7-Iodo-Trp, D-6,7- | |
| | methyl-Trp, D-6,7-cyano-Trp, D-6,7- | |
| | hydroxy-Trp, or D-6,7-NO₂-Trp; | |
| | X⁵ is (S)-2-amino-2-methylhept-6- | |
| | ynoic acid; | |
| | X⁷ is D-p-Fluoro-Phe, D-p-Chloro-Phe, | |
| | D-p-Bromo-Phe, D-p-Iodo-Phe, D-p- | |
| | methyl-Phe, D-p-cyano-Phe, D-p- | |
| | hydroxy-Phe, D-p-NO₂-Phe, or D-p- | |
| | CF₃-Phe; | |
| | X⁹ is D-Lys or D-Gln; | |
| | X¹¹ is D-Leu; | |
| | X¹² is (R)-2-amino-2-methyloct-7- | |
| | ynoic acid; | |
| | the N-terminus is optionally conjugated | |
| | to an acyl group having the formula | |
| | RCO−; R is an alkane having the | |
| | formula CₙH₂ₙ₊₁; n is an integer | |
| | from 1 to 10; | |
| | the C-terminal amino acid optionally | |
| | comprises an amino group; | |
| | amino acids 1-12 are D amino acids; | |
| | and the staple is a di-alkyne obtained | |
| | through alkyne cross-coupling between | |
| | X⁵ and X¹² | |
| 20 | Polypeptide tail | AAAAAA |
| | All D-amino acids | |
| 21 | Polypeptide tail | AFAAAA |
| | All D-amino acids | |
| 22 | Polypeptide tail | EAAAAA |
| | All D-amino acids | |
| 23 | Polypeptide tail | AAAAAAAAA |
| | All D-amino acids | |
| 24 | ATSP-7041 wherein | LTFXEYWAQXXSAA |
| | X⁴ is (R)-2-amino-2-methyldec-9-enoic | |
| | acid; | |
| | X¹⁰ is cyclo-butyl alanine (cba); | |
| | X¹¹ is (S)-2-amino-2-methylhept-6- | |
| | enoic acid; | |
| | Amino acids 1-14 are L-amino acids; | |
| | X⁴ and X¹¹ are linked together in an | |
| | olefin linkage; | |
| | the N-terminus is conjugated to an | |
| | acetyl group; | |
| | the C-terminal amino acid comprises an | |
| | amino group; | |
| 25 | DPMI delta (5-12 olefin staple)-NH₂ | TAXYXNXEKLLXAAAAAA |
| | wherein | |
| | X³ is D-6-Fluoro-Trp; | |
| | X⁵ is (S)-2-amino-2-methyldec-9-enoic | |
| | acid; | |
| | X⁷ is D-p-CF₃-Phe; | |
| | X¹² is (R)-2-amino-2-methylhept-6- | |
| | enoic acid; | |
| | Amino acids 1-18 are D-amino acids; | |
| | X⁵ and X¹² are linked together an olefin | |
| | linkage; | |
| | the N-terminus is conjugated to an | |
| | acetyl group; | |
| | the C-terminal amino acid comprises an | |
| | amino group; | |
| 26 | Peptide 13 wherein | RFMDYWEGL |
| | N-terminal FAM; | |
| | C-terminal amide; | |
| 27 | A 5-12 olefin stapled p53 | TAXYXNXEXLXXXXXXXXXXX |
| | peptidomimetic macrocycle wherein | |
| | X³ is D-Trp, D-6-Fluoro-Trp, D-6- | |
| | Chloro-Trp, D-6-Bromo-Trp, D-6- | |
| | Iodo-Trp, D-6-methyl-Trp, D-6-cyano- | |
| | Trp, D-6-hydroxy-Trp, D-6-NO₂-Trp, | |
| | D-7-Fluoro-Trp, D-7-Chloro-Trp, D-7- | |
| | Bromo-Trp, D-7-Iodo-Trp, D-7- | |
| | methyl-Trp, D-7-cyano-Trp, D-7- | |
| | hydroxy-Trp, D-7-NO₂-Trp, D-6,7- | |
| | Fluoro-Trp, D-6,7-Chloro-Trp, D-6,7- | |
| | Bromo-Trp, D-6,7-Iodo-Trp, D-6,7- | |
| | methyl-Trp, D-6,7-cyano-Trp, D-6,7- | |
| | hydroxy-Trp, or D-6,7-NO₂-Trp; | |
| | X⁵ is (S)-2-amino-2-methyldec-9-enoic | |
| | acid; | |
| | X⁷ is D-p-Fluoro-Phe, D-p-Chloro-Phe, | |
| | D-p-Bromo-Phe, D-p-Iodo-Phe, D-p- | |
| | methyl-Phe, D-p-cyano-Phe, D-p- | |
| | hydroxy-Phe, D-p-NO₂-Phe, or D-p- | |
| | CF₃-Phe; | |
| | X⁹ is D-Lys or D-Gln; | |
| | X¹¹ is D-Leu; | |
| | X¹² is (R)-2-amino-2-methylhept-6- | |
| | enoic acid; | |
| | X¹³ is D-Ala, D-Glu, D-Gla (gamma- | |
| | carboxylic glutamic acid); | |
| | X¹⁴ is D-Ala; | |
| | X¹⁵ is D-Ala, D-Phe, or D-Glu; | |
| | X¹⁶ is D-Ala; | |
| | X¹⁷ is D-Ala or D-alpha-methyl-Glu; | |
| | X¹⁸ is D-Ala; | |
| | X¹⁹ is D-Ala or absent; | |
| | X²⁰ is D-Ala or absent; | |
| | X²¹ is D-Ala or absent; | |
| | the N-terminus amino group of the Thr | |
| | at position 1 is conjugated to an acyl | |
| | group having the formula RCO−; R is | |
| | an alkane having the formula | |
| | CₙH₂ₙ₊₁; n is an integer from 1 to 10; | |
| | the C-terminal amino acid optionally | |
| | comprises an amino group; amino acids | |
| | 1-21 are D amino acids; | |
| | and the staple is an olefin obtained | |
| | through ring-closing metathesis | |
| | between X⁵ and X¹² | |

### REFERENCES CITED HEREIN AND WHICH ARE INCORPORATED HEREIN BY REFERENCE

1. Petta I., Lievens S., Libert C., Tavernier J., De Bosscher K. Modulation of Protein-Protein Interactions for the Development of Novel Therapeutics. Mol. Ther. 2015; 24:707-718.
2. Macalino S. J. Y., Basith S., Clavio N. A. B., Chang H., Kang S., Choi S. Evolution of In Silico Strategies for Protein-Protein Interaction Drug Discovery. Molecules. 2018; 23:1963
3. Scott, D. E., Bayly, A. R., Abell, C. and Skidmiore, J. (2016) Small molecules, big targets: drug discovery faces the protein-protein interaction challenge. Nat. Rev. Drug Discov. 15, 533-550
4. Nevola L, Giralt E. Modulating protein-protein interactions: the potential of peptides. Chem Commun. 2015:51:3302-15.
5. Lau, J. L.; Dunn, M. K. Therapeutic peptides: Historical perspectives, current development trends, and future directions Bioorg. Med. Chem. 2017, 26, 2700-2707
6. Fosgerau, K.; Hoffinann, T. Peptide therapeutics: current status and future directions Drug Discovery Today 2015, 20, 122-128
7. Bakail M., Ochsenbein F. Targeting protein-protein interactions, a wide open field for drug design. Comptes Rendus Chimie. 2016, 19, 19-27
8. A. Henninot, J. C. Collins, J. M. Nuss. The current state of peptide drug discovery: back to the future J. Med. Chem., 61 (2018), pp. 1382-1414
9. Morrison C., Constrained peptides' time to shine?. Nature Reviews Drug Discovery volume 17, pages 531-533 (2018)
10. Valeur E., et al. New Modalities for Challenging Targets in Drug Discovery. Angew. Chem. Int. Ed. 2017, 56, 10294-10323
11. Cary, D. R.; Ohuchi, M.; Reid, P. C.; Masuya, K. Constrained Peptides in Drug Discovery and Development. Yuki Gosei Kagaku Kyokaishi 2017, 75, 1171-1178,
12. Vinogradov A., Macrocyclic Peptides as Drug Candidates: Recent Progress and Remaining Challenges. J. Am. Chem. Soc., 2019, 141 (10), pp 4167-4181.
13. Sawyer, T. K. Macrocyclic a helical peptide therapeutic modality: A perspective of leamings and challenges. Bioorg. Med. Chem. 2018, 26, 2807-2815
14. L. D. Walensky, G. H. Bird Hydrocarbon-stapled peptides: principles, practice, and progress J Med Chem, 57 (2014), pp. 6275-6288,
15. Y. S. Tan, D. P. Lane, C. S. Verma Stapled peptide design: principles and roles of computation Drug Discov Today, 21 (2016), pp. 1642-1653
16. Ali A M., et al. Stapled Peptides Inhibitors: A New Window for Target Drug Discovery. Computational and Structural Biotechnology Journal, 2019, 17, 263-281.
17. Klein M., Stabilized helical peptides: overview of the technologies and its impact on drug discovery. Expert Opinion on Drug Discovery. 2017, 12, 1117-1125.
18. Lerge J et al. Stapled peptides as a new technology to investigate protein-protein interactions in human platelets. Chem Sci, 2108, 9, 4638-4643.
19. Xu W., Macrocyclized Extended Peptides: Inhibiting the Substrate-Recognition Domain of Tankyrase. J. Am. Chem. Soc., 2017, 139 (6), pp 2245-2256
20. Wiedmann, M M., et al. Development of Cell-Permeable, Non-Helical Constrained Peptides to Target a Key Protein-Protein Interaction in Ovarian Cancer. Angew. Chem. Int. Ed. 2017, 56, 524-529
21. L. D. Walensky, A. L. Kung, I. Escher, T. J. Malia, S. Barbuto, R. D. Wright, G. Wagner, G. L. Verdine and S. J. Korsmeyer. Activation of apoptosis in vivo by a hydrocarbon-stapled BH3 helix. Science, 2004, 305, 1466-1470.
22. S. A. Kawamoto, A. Coleska, X. Ran, H. Yi, C. Y. Yang and S. Wang. Design of triazole-stapled BCL9 α-helical peptides to target the β-catenin/B-cell CLI/lymphoma 9 (BCL9) protein-protein interaction, J. Med. Chem., 2012, 55, 1137-1146
23. K. Takada, Di Zhu, G. H. Bird, K. Sukhdeo, J.-J. Zhao, M. Mani, M. Lemieux, D. E. Carrasco, J. Ryan,
24. D. Horst, M. Fulciniti, N. C. Munshi, W. Xu, A. L. Kung, R. A. Shivdasani, L. D. Walensky and D. R. Carrasco. Targeted disruption of the BCL9/β-catenin complex inhibits oncogenic Wnt signaling. Sci. Transl. Med., 2012, 4, 148ra117.
25. L Dietrich, B Rathmer, K Ewan, T Bange, S Heinrichs, TC Dale, D Schade, TN Grossmann. Cell permeable stapled peptide inhibitor of Wnt signaling that targets β-catenin protein-protein interactions. Cell Chem. Biol., 24, 958-968 (2017).
26. Spiegel J, Cromm P M, Itzen A, Goody R S, Grossmann T N, Waldmann H. Direct targeting of Rab-GTPase-effector interactions. Angew Chem Int Ed Engl. 2014 Feb. 24; 53(9):2498-503
27. Xie M., et al. Structural Basis of Inhibition of ERα-Coactivator Interaction by High-Affinity N-Terminus Isoaspartic Acid Tethered Helical Peptides. J. Med. Chem., 2017, 60 (21), pp 8731-8740.
28. Paola de., et al. Cullin3-BTB interface: a novel target for stapled peptides. PLoS One. 2015 Apr. 7; 10(4):e0121149.
29. Misawa T., et al. Structural development of stapled short helical peptides as vitamin D receptor (VDR)-coactivator interaction inhibitors. Bioorg Med Chem. 2015 Mar. 1; 23(5):1055-61.
30. Lama D., et al, Structural insights reveal a recognition feature for tailoring hydrocarbon stapled-peptides against the eukaryotic translation initiation factor 4E protein. Chemical Science. 2019, 10, 2489-2500
31. F. Bemal, A. F. Tyler, S. J. Korsmeyer, L. D. Walensky and G. L. Verdine. Reactivation of the p53 tumor suppressor pathway by a stapled p53 peptide. J. Am. Chem. Soc., 2007, 129, 2456-2457.
32. L. K. Henchey, J. R. Porter, I. Ghosh and P. S. Arora. High specificity in protein recognition by hydrogen-bond-surrogate a-helices: selective inhibition of the p53/MDM2 complex. Chembiochem., 2010, 11, 2104-2107
33. C. J. Brown, S. T., Quah, J. Jong, A. M. Goh, P. C., Chiam, K. H. Khoo, M. L. Choong, M. A. Lee, L. Yurlova, K. Zolghadr, T. L. Joseph, C. S. Verma and D. P. Lane. Stapled peptides with improved potency and specificity that activate p53. ACS Chem. Biol., 2013, 8, 506-512
34. Y. S. Chang, B. Graves, V. Guerlavais, C. Tovar, K. Packman, T. To, K. Olson, K. Kesavan, P. Gangurde, A. Mukherjee, T. Baker, K. Darlak, C. Elkin, Z. Filipovic, F. Z Qureshi, H. Cai, P. Berry, E. Feyfant, X. E. Shi, J. Horstick, A. Annis, N. Fotouhi, T. Manning, H. Nash, L. T. Vassilev and T. K. Sawyer. Stapled a-helical peptide drug development: a potent dual inhibitor of MDM2 and MDMX for p53-dependent cancer therapy. Proc. Natl. Acad. Sci. U.S.A, 2013, 110, E3445-E3455.
35. A. Burgess, K. M. Chia, S. Haupt, D. Thomas, Y. Haupt and E. Lim. Clinical overview of MDM2/Xtargeted therapies. Front. Oncol., 2016, 6, 1-7.
36. K. Kojima, J. Ishizawa and M. Andreeff. Pharmacological activation of wild-type p53 in the therapy of leukemia. Exp. Hematol., 2016, 44, 791-798.
37. V. Tisato, R. Voltan, A. Gonelli, P. Secchiero and G. Zauli. MDM2/X inhibitors under clinical evaluation: Perspectives for the management of hematological malignancies and pediatric cancer. J. Hematol. Oncol., 2017, 10, 133.
38. Cromm P M. Et al. Protease-Resistant and Cell-Permeable Double-Stapled Peptides Targeting the Rab8a GTPase. ACS Chem. Biol., 2016, 11 (8), pp 2375-2382
39. Bird G H., et al., Hydrocarbon double-stapling remedies the proteolytic instability of a lengthy peptide therapeutic. PNAS Aug. 10, 2010 107 (32) 14093-14098
40. Gaillard V., et al. A Short Double-Stapled Peptide Inhibits Respiratory Syncytial Virus Entry and Spreading. Antimicrobial Agents and Chemotherapy March 2017, 61 (4) e02241-16
41. M. Liu, M. Pazgier, C. Li, W. Yuan, C. Li, W. Lu Angew. Chem., Int. Ed., 49 (2010), p. 3649
42. M. Liu, C. Li, M. Pazgier, C. Li, Y. Mao, Y. Lv, B. Gu, G. Wei, W. Yuan, C. Zhan, W. Y. Lu, W. Lu Proc. Natl. Acad. Sci. USA, 107 (2010), p. 14321
43. K. Mandal, M. Uppalapati, D. Ault-Richd, J. Kenney, J. Lowitz, S. S. Sidhu, S. B. Kent Proc. Natl. Acad. Sci. USA, 109 (2012), p. 14779
44. H. N. Chang, B. Y. Liu, Y. K. Qi, Y. Zhou, Y. P. Chen, K. M. Pan, W. W. Li, X. M. Zhou, W. W. Ma, C. Y. Fu, Y. M. Qi, L. Liu, Y. F. Gao Angew. Chem., Int. Ed., 54 (2015), p.
45. Welch B D, Francis J N, Redman J S, Paul S, Weinstock M T, Reeves J D, Lie Y S, Whitby F G, Eckert D M, Hill C P, Root M J, Kay M S. Design of a potent D-peptide HIV-1 entry inhibitor with a strong barrier to resistance. J Virol. 2010; 84(21):11235-44.
46. Lane D P, p53, guardian of the genome Nature. 1992 Jul. 2; 358(6381):15-6.
47. Karni-Schmidt, O., M. Lokshin, and C. Prives. 2016. The roles of MDM2 and MDMX in cancer. Annu. Rev. Pathol. 11:617-644
48. Dongsheng Pei,1,2 Yanping Zhang, 1,2 and Junnian Zhengl Regulation of p53: a collaboration between MDM2 and MDMX. Oneggt. 2012 March; 3(3): 228-235.
49. Tisato V1, Voltan R2, Gonelli A2, Secchiero P2, Zauli G2. MDM2/X inhibitors under clinical evaluation: perspectives for the management of hematological malignancies and pediatric cancer. J Hematol Oncol. 2017 Jul. 3; 10(1):133
50. F. Funda Meric-Bemstam, M. S. Saleh, J. R. Infante, S. Goel, G. S. Falchook, G. Shapiro, K. Y. Chung, R. M. Conry, D. S. Hong, J. S. Wang, U. Steidl, L. D. Walensky, V. Guerlavais, M. Payton, D. A. Annis, M. Aivado, M. R. Patel Phase I trial of a novel stapled peptide ALRN-6924 disrupting MDMX- and MDM2-mediated inhibition of WT p53 in patients with solid tumors and lymphomas. J. Clin. Oncol. 35, 2505-2505 (2017)
51. Zhan, C.; Zhao, L.; Wei, X.; Wu, X.; Chen, X.; Yuan, W.; Lu, W. Y.; Pazgier, M.; Lu, W. An ultrahigh affinity d-peptide antagonist of MDM2 J. Med. Chem. 2012, 55, 6237-6241
52. C J Brown, S T Quah, J Jong, A M Goh, P C Chiam, K H Khoo, M L Choong, et al. Stapled peptides with improved potency and specificity that activate p53. ACS chemical biology 8 (3), 506-512
53. Tan, Y. S. et al. Benzene Probes in Molecular Dynamics Simulations Reveal Novel Binding Sites for Ligand Design. JPhys Chem Lett 7, 3452-3457,
54. D. Thean, J. S. Ebo, T. Luxton, Xue'Er Cheryl Lee, T. Y. Yuen, F. J. Ferrer, C. W. Johannes, D. P. Lane & C. J. Brown. Enhancing Specific Disruption of Intracellular Protein Complexes by Hydrocarbon Stapled Peptides Using Lipid Based Delivery. Scientific Reportsvolume 7, Article number: 1763 (2017).
55. Kannan S, Zacharias M (2007) Enhanced sampling of peptide and protein conformations using replica exchange simulations with a peptide backbone biasing-potential. Proteins. 66: 697-706.
56. Ostermeir K, Zacharias M. Hamiltonian replica-exchange simulations with adaptive biasing of peptide backbone and side chain dihedral angles. J. Comput. Chem., 35 (2014), pp
57. Schafmeister, C. E.; Po, J.; Verdine, G. L. An all-hydrocarbon cross-linking system for enhancing the helicity and metabolic stability of peptides. J. Am. Chem. Soc. 2000, 122, 5891-5892. 150-158
58. LD Walensky, GH bird, Hydrocarbon-Stapled Peptides: Principles, Practice, and Progress. J. Med. Chem. 2014, 57, 6275-6288
59. Y. S. Chang, B. Graves, V. Guerlavais, C. Tovar, K. Packman, T. To, K. Olson, K. Kesavan, P. Gangurde, A. Mukherjee, T. Baker, K. Darlak, C. Elkin, Z. Filipovic, F. Z Qureshi, H. Cai, P. Berry, E. Feyfant, X. E. Shi, J. Horstick, A. Annis, N. Fotouhi, T. Manning, H. Nash, L. T. Vassilev and T. K. Sawyer. Stapled a-helical peptide drug development: a potent dual inhibitor of MDM2 and MDMX for p53-dependent cancer therapy. Proc. Natl. Acad. Sci. U.S.A, 2013, 110, E3445-E3455.
60. A. Furukawa, C. E. Townsend, J. Schwochert, C. R. Pye, M. A. Bednarek, R. S. Lokey. Passive membrane permeability in cyclic peptomer scaffolds is robust to extensive variation in side chain functionality and backbone geometry. J Med Chem, 59 (2016), pp. 9503-9512
61. Li Y C, et al. A versatile platform to analyze low-affinity and transient protein-protein interactions in living cells in real time. Cell Rep. 2014; 9:1946-58
62. Bird G H, et al. Mucosal delivery of a double-stapled RSV peptide prevents nasopulmonary infection. J Clin Invest. 2014; 124:2113-24
63. Thomas J C, Cooper J M, Clayton N S, Wang C, White M A, Abell C, Owen D, and Mott H R (2016) Inhibition of Ral GTPases using a stapled peptide approach. J Biol Chem 291:18310-18325
64. P. M. Cromm, J. Spiegel, P. Küchler, L. Dietrich, J. Kriegesmann, M. Wendt, R. S. Goody, H. Waldmann and T. N. Grossmann, ACS Chem. Biol., 2016, 11, 2375-2382
65. Speltz T. E., Mayne C. G., Fanning S. W., Siddiqui Z., Tajkhorshid E., Greene G. L. A “cross-stitched” peptide with improved helicity and proteolytic stability. Org Biomol Chem. 2018; 16:3702-3706
66. Kawamoto S. A., Coleska A., Ran X., Yi H., Yang C. Y., Wang S. Design of triazole-stapled BCL9 α-helical peptides to target the 0-catenin/B-cell CLLlymphoma 9 (BCL9) protein-protein interaction. J Med Chem. 2012; 55:1137-1146.
67. Hilinski G. J., Kim Y.-W., Hong J., Kutchukian P. S., Crenshaw C. M., Berkovitch S. S. et al. 2014. Stitched α-helical peptides via bis ring-closing metathesis. J Am Chem Soc. 2014 Sep. 3; 136(35):12314-22.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A p53 peptidomimetic macrocycle, comprising:
(a) an i, i+4 olefin staple and a polypeptide tail covalently linked at its N-terminus to the C-terminal amino acid of the p53 peptidomimetic macrocycle;
(b) an i, i+7 olefin staple and a polypeptide tail covalently linked at its N-terminus to the C-terminal amino acid of the p53 peptidomimetic macrocycle; or
(c) an i, i+7 di-alkyne staple and optionally a polypeptide tail covalently linked at its N-terminus to the C-terminal amino acid of the p53 peptidomimetic macrocycle,
wherein the p53 peptidomimetic macrocycle comprises all D-configuration amino acids and the polypeptide tail comprises three to nine amino acids, each amino acid of the polypeptide tail independently having a D-configuration or an L-configuration, or each amino acid in the polypeptide tail having a D-configuration.

2. The p53 peptidomimetic macrocycle of claim 1, wherein the p53 peptidomimetic macrocycle comprises an i, i+7 di-alkyne staple and a polypeptide tail covalently linked at its N-terminus to the C-terminal amino acid of the p53 peptidomimetic macrocycle.

3. The p53 peptidomimetic macrocycle of claim 1, wherein the p53 peptidomimetic macrocycle comprises 12 amino acids and an i, i+4 olefin staple formed between the α-carbons of two α,α-disubstituted amino acids located at amino acid positions 6 and 10 of the p53 peptidomimetic macrocycle and a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration or, in specific embodiments, each amino acid in the polypeptide tail having a D-configuration.

4. The p53 peptidomimetic macrocycle of claim 3, wherein the α,α-disubstituted amino acids at amino acid positions 6 and 10 of the p53 peptidomimetic macrocycle comprise (R)-2-amino-2-methylhept-6-enoic acid.

5. The p53 peptidomimetic macrocycle of claim 4, wherein the p53 peptidomimetic macrocycle further comprises D-6-fluoro-tryptophane at amino acid position 3 and D-p-CF3-phenylalanine at amino acid position 7.

6. The p53 peptidomimetic macrocycle of claim 5, wherein the p53 peptidomimetic macrocycle further comprises threonine at amino acid position 1, alanine at amino acid position 2, tyrosine at amino acid position 4, alanine at amino acid position 5, glutamic acid at amino acid position 8, lysine or glutamine at amino acid position 9, leucine at amino acid position 11, and arginine or serine at amino acid position 12.

7. The p53 peptidomimetic macrocycle of claim 6, wherein the p53 peptidomimetic macrocycle comprises glutamine at amino acid at position 9 and serine at amino acid at position 12.

8. The p53 peptidomimetic macrocycle of claim 1, wherein the p53 peptidomimetic macrocycle comprises 12 amino acids and an i, i+7 di-alkyne staple formed between the α-carbons of two α,α-disubstituted amino acids located at amino acid positions 5 and 12 of the p53 peptidomimetic macrocycle, and optionally a polypeptide tail covalently linked at its N-terminus to the C-terminal amino acid of the p53 peptidomimetic macrocycle, wherein the p53 peptidomimetic macrocycle comprises all D-configuration amino acids and the polypeptide tail comprises three to nine amino acids, each amino acid of the polypeptide tail independently having a D-configuration or an L-configuration.

9. The p53 peptidomimetic macrocycle of claim 8, wherein the α,α-disubstituted amino acid at amino acid position 5 of the p53 peptidomimetic macrocycle comprises (S)-2-amino-2-methylhept-6-ynoic acid and the α,α-disubstituted amino acid at amino acid position 12 of the p53 peptidomimetic macrocycle comprises (R)-2-amino-2-methyloct-7-ynoic acid.

10. The p53 peptidomimetic macrocycle of claim 9, wherein the p53 peptidomimetic macrocycle further comprises D-6-fluoro-tryptophane at amino acid position 3 of the p53 peptidomimetic macrocycle and D-p-CF3-phenylalanine at amino acid position 7 of the p53 peptidomimetic macrocycle.

11. The p53 peptidomimetic macrocycle of claim 10, wherein the p53 peptidomimetic macrocycle further comprises threonine at amino acid at position 1, alanine at amino acid position 2, tyrosine at amino acid position 4, asparagine at amino acid position 6, glutamic acid at amino acid position 8, lysine or glutamine at amino acid position 9, leucine at amino acid position 10, and leucine at amino acid position 11.

12. The p53 peptidomimetic macrocycle of claim 11, wherein the p53 peptidomimetic macrocycle comprises glutamine at amino acid position 9.

13. The p53 peptidomimetic macrocycle of claim 1, wherein the p53 peptidomimetic macrocycle comprises 12 amino acids and an i, i+7 olefin staple formed between the α-carbons of two α,α-disubstituted amino acids located at amino acid positions 5 and 12 of the p53 peptidomimetic macrocycle, and optionally a polypeptide tail, wherein the p53 peptidomimetic macrocycle comprises all D-configuration amino acids and the polypeptide tail comprises three to nine amino acids, each amino acid of the polypeptide tail independently having a D-configuration or an L-configuration.

14. The p53 peptidomimetic macrocycle of claim 13, wherein the α,α-disubstituted amino acid at amino acid position 5 of the p53 peptidomimetic macrocycle comprises (S)-2-amino-2-methyldec-9-enoic acid and the α,α-disubstituted amino acid at amino acid position 12 of the p53 peptidomimetic macrocycle comprises (R)-2-amino-2-methylhept-6-enoic acid.

15. The p53 peptidomimetic macrocycle of claim 14, wherein the p53 peptidomimetic macrocycle further comprises D-6-fluoro-tryptophane at amino acid position 3 of the p53 peptidomimetic macrocycle and D-p-CF3-phenylalanine at amino acid position 7 of the p53 peptidomimetic macrocycle.

16. The p53 peptidomimetic macrocycle of claim 15, wherein the p53 peptidomimetic macrocycle further comprises threonine at amino acid at position 1, alanine at amino acid position 2, tyrosine at amino acid position 4, asparagine at amino acid position 6, glutamic acid at amino acid position 8, lysine or glutamine at amino acid position 9, leucine at amino acid position 10, and leucine at amino acid position 11.

17. The p53 peptidomimetic macrocycle of claim 16, wherein the p53 peptidomimetic macrocycle comprises glutamine at amino acid position 9.

18-38. (canceled)

39. A composition comprising the p53 peptidomimetic macrocycle of claim 1 and a pharmaceutically acceptable carrier.

40. A method for treating cancer in a subject in need thereof comprising administering to the subject the p53 peptidomimetic macrocycle of claim 1.

41-53. (canceled)

54. A combination therapy for treating cancer comprising administering to a subject in need thereof a therapeutically effective amount of the p53 peptidomimetic macrocycle of claim 1 and a therapeutically effective dose of a chemotherapy agent or radiation or a therapeutically effective amount of a checkpoint inhibitor.

55-56. (canceled)

57. A treatment for cancer comprising administering to a subject having the cancer a vector comprising a nucleic acid molecule encoding a wild-type p53 or p53 variant or analog with transcriptional activation activity followed by one or more administrations of a therapeutically effective amount of the p53 peptidomimetic macrocycle of claim 1.

58-60. (canceled)
